(19) United States
(12) Patent Application Publication
(10) Pub. No.: US 2025/0176904 A1
(43) Pub. Date: Jun. 5, 2025

(54) PRE-DOSING METHOD OF ASSESSING DRUG BENEFITS IN PATIENTS

(71) Applicant: Diamond Therapeutics Inc., Toronto (CA)

(72) Inventors: Judith BLUMSTOCK, Toronto (CA); William James TYLER, Chelsea, AL (US)

(21) Appl. No.: 18/841,530

(22) PCT Filed: May 22, 2023
(86) PCT No.: PCT/IB2023/000292

§ 371 Date: Aug. 26, 2024

Related U.S. Application Data
(60) Provisional application No. 63/387,769, filed on Dec. 16, 2022.
(60) Provisional application No. 63/344,939, filed on May 23, 2022.

(51) Int. Cl. A61B 5/00; A61B 5/16; A61K 31/675; A61K 45/06; G01N 33/68

(52) U.S. Cl. CPC A61B 5/4839; A61B 5/165; A61K 31/675; A61K 45/06; G01N 33/6896; G01N 2800/52

(57) ABSTRACT

Disclosed herein are methods of treatment and methods of evaluating individuals to identify and measure biomarkers and/or states of the individuals indicative of drug responsiveness to one or more 5-HT receptor agonist.

### CROSS-REFERENCE

This application claims benefit of U.S. Provisional Patent Application No. 63/344,939 filed on May 23, 2022 and U.S. Provisional Patent Application No. 63/387,769 filed on Dec. 16, 2022, each incorporated herein by reference in its entirety.

### BACKGROUND

Without pre-dosing assessment, it is difficult to predict which patients will benefit from drug treatment, in particular, 5HT receptor agonist therapy useful in the treatment of mental diseases, disorders or conditions. It would be helpful for doctors, clinicians, insurers and the like to identify those patients that would most favorably respond to drug treatment.

### SUMMARY

Provided in certain embodiments herein is a method of assessing an individual's responsiveness to a drug therapy prior to the administration of an active agent. In some embodiments, provided herein is a method of identifying a beneficial response of an individual to drug treatment of a mental disease, disorder or condition prior to administration. In some instances, methods provided herein are useful for assessing the benefit of medical or personalized care in an individual by determining if the individual presents markers indicative of responsiveness to drug therapy. For example, in some instances, the methods provided herein facilitate the identification of a biomarker(s) associated with a therapeutic outcome. In some embodiments, the therapeutic outcome includes the treatment of a mental disease, disorder or condition with a 5HT receptor agonist (e.g. psilocybin). In some embodiments, the methods are predictive of therapeutic effectiveness of the treatment of an individual with a drug.

In some embodiments, provided herein is a method for identifying a biomarker in an individual prior to administration of a drug. In some embodiments, the method comprises identifying or measuring a state of an individual by performing an evaluation (e.g., pre-dosing) on the individual. In some embodiments, the effectiveness of drug therapy (e.g. the active agent) is determined (at least in part) by the identification or measurement of the evaluation. In some embodiments, the method comprises: 1) identifying or measuring the state of the individual by performing an evaluation (e.g., pre-dosing) on the individual, and 2) administering an active agent based on the identification or measurement of the evaluation.

Provided herein is a method of assessing and treating an individual comprising:
a) identifying or measuring a biomarker of the individual by performing an evaluation on the individual;
b) assessing the biomarker;
c) determining (e.g., predicting) the biomarker is indicative of drug responsiveness based at least in part of the assessing in step (b); and
d) administering to the individual an active agent (e.g. 5-HT receptor agonist (e.g., psilocybin)).

Provided herein is a method of assessing and treating an individual comprising:
a. identifying or measuring a state (e.g., a physiological parameter, such as brain activity or a psychological parameter, such as an emotional state, e.g. mood) of the individual by performing an (e.g., physiological, such as brain activity or a psychological parameter, such as an emotional state, e.g. mood) evaluation (e.g., an electroencephalogram (EEG) or written or verbal questionnaire) on the individual;
b. assessing the measured state;
c. determining (e.g., predicting) the measured state is indicative of drug responsiveness based at least in part on the assessing in step (b); and
d. administering to the individual an active agent (e.g. 5-HT receptor agonist (e.g., psilocybin)).

Provided herein is a method of assessing and treating an individual comprising:
a. identifying or measuring a state (e.g., a physiological parameter, such as brain activity or a psychological parameter, such as an emotional state, e.g. mood) of the individual by performing an (e.g., physiological, such as brain activity or a psychological parameter, such as an emotional state, e.g. mood) evaluation (e.g., an electroencephalogram (EEG) or written or verbal questionnaire) on the individual;
b. assessing the measured state;
c. identifying a biomarker indicative of drug responsiveness based at least in part on the assessing in step (b); and
d. administering to the individual an active agent (e.g. 5-HT receptor agonist (e.g., psilocybin)).

Provided herein is a method of assessing an individual's responsiveness to drug therapy prior to the administration of an active agent, comprising:
a. identifying or measuring a state (e.g., a physiological parameter, such as brain activity or a psychological parameter, such as an emotional state, e.g. mood) of the individual by performing an (e.g., physiological, such as brain activity or a psychological parameter, such as an emotional state, e.g. mood) evaluation (e.g., an electroencephalogram (EEG) or written or verbal questionnaire) on the individual;
b. assessing the measured state;
c. determining (e.g., predicting) the measured state is indicative of drug responsiveness based at least in part on the assessing in step (b).

In some embodiments, treating comprises treating a (e.g. a mental, a behavioral, or a neuropsychiatric) disease, disorder or condition of the individual. In some embodiments, assessing comprises predicting a therapeutic effectiveness of the treatment of the individual with the active agent.

In some embodiments, the state of the individual comprises a physiological, neurological, psychological, metabolic, or biologic state or a combination thereof. In some embodiments, the state of the individual is measured or identified by a brain activity (e.g. using a brain evaluation (e.g. EEG, MEG, fNRIS, PET transcranial functional ultrasound)), a heart test (e.g. evaluated using an EEG for recording heart rate or EKG for recording heart rhythm), a visual test, an auditory test, a biological sample (e.g. for evaluating changes in serum, plasma, whole blood, urine, sweat or the like), patient reporting (e.g. through written or verbal questionnaires or surveys to evaluate mood, affect, coping, sleep quality, stress, anxiety, memory, and/or other emotions), functional data (e.g. evaluated by brain imaging (e.g. fNRIS, PET) or monitoring of brain activity levels (e.g. EEG)), body temperature, food intake, metabolic rate, perspiration, hydration, salivation, pupil dilation, breathing rate, pulse rate, skin colour, or skin temperature, and the like.

In some embodiments, the biomarker is used to identify high and low responding individuals. In some embodiments, the biomarker is used to identify treatable and non-treatable individuals. In some embodiments, wherein the biomarker is used to identify individuals who would benefit most from treatment with the active agent. In some embodiments, the biomarker is used to identify and/or predict personalized medical treatment with the active agent.

In some embodiments, the disease, disorder or condition comprises a brain disease, disorder or condition (e.g., a mental, a behavioral, a neuropsychiatric, or the like, condition).

In some embodiments, treating comprises treating or reducing the incidence of an episode in an individual susceptible to or suffering from a brain disease, disorder or condition (e.g., a mental, a behavioral, a neuropsychiatric, or the like, condition) by administering a dose (e.g., a therapeutically effective dose) of the active agent (e.g., one or more 5-HT receptor agonist (e.g., psilocybin)) to the individual.

In some embodiments, the dose (e.g., therapeutically effective dose) of the active agent (e.g., one or more 5-HT receptor agonist (e.g., psilocybin)) comprises about 0.5 mg to about 5 mg of the active agent (e.g., one or more 5-HT receptor agonist (e.g., psilocybin)). In some embodiments, the dose (e.g., therapeutically effective dose) of the active agent (e.g., one or more 5-HT receptor agonist (e.g., psilocybin)) comprises about 3 mg of the active agent (e.g., one or more 5-HT receptor agonist (e.g., psilocybin)).

In some embodiments, the dose (e.g., therapeutically effective dose) of the active agent (e.g., one or more 5-HT receptor agonist (e.g., psilocybin)) is administered to an individual once daily. In some embodiments, the dose (e.g., therapeutically effective dose) of the active agent (e.g., one or more 5-HT receptor agonist (e.g., psilocybin)) is administered to an individual once every 3 days. In some embodiments, the dose (e.g., therapeutically effective dose) of the active agent (e.g., one or more 5-HT receptor agonist (e.g., psilocybin)) is administered to an individual for 28 days or less. In some embodiments, the dose (e.g., therapeutically effective dose) of the active agent (e.g., one or more 5-HT receptor agonist (e.g., psilocybin)) is administered to an individual for at least 28 days.

In some embodiments, the active agent (e.g., one or more 5-HT receptor agonist (e.g., psilocybin)) is formulated for oral administration.

In some embodiments, the active agent (e.g., one or more 5-HT receptor agonist (e.g., psilocybin)) is formulated as a solution at a concentration of about 0.05 mg/mL to about 1 mg/mL. In some embodiments, the active agent (e.g., one or more 5-HT receptor agonist (e.g., psilocybin)) is formulated as a solution at a concentration of about 0.5 mg/mL.

In some embodiments, the evaluation is performed by the individual (e.g., in or outside a healthcare setting (e.g., in a natural environment, such as, for example, at home, at work, or the like)). In some embodiments, the evaluation is performed by a heath care provider (e.g., doctor, nurse, clinician). In some embodiments, the evaluation is performed using an application (e.g., mobile application).

In some embodiments, identifying in (a) is performed prior to the administration of the active agent.

In some embodiments, the evaluation (e.g. brain test) is performed using a wearable device (e.g brain imaging).

In some embodiments, prior to step (a), the method comprises obtaining a biological sample from the individual.

In some embodiments, the identifying step comprises measuring the biological sample obtained from the subject. In some embodiments, the biological sample comprises serum, plasma, whole blood, urine, or the like.

In some embodiments, two or more of any step are performed simultaneously. In some embodiments, two or more of any step are performed sequentially.

In some embodiments, the identifying or measuring a state is performed by administering (e.g., to the individual) a questionnaire (e.g. STAI).

In some embodiments, the identifying or measuring a state is performed through measurement of pupil dilation in the individual.

In some embodiments, a test dose of the active agent (e.g. an amount used to assist in measuring of a biomarker (e.g. pupil dilation)) is administered to an individual prior to the method.

In certain embodiments, the state of an individual can be a physiological, neurological, psychological, metabolic, or biological state or a combination thereof. In some embodiments the state of an individual is measured or identified by a brain activity (e.g. using a brain evaluation (e.g. EEG, MEG, fNRIS, PET transcranial functional ultrasound)), a heart test (e.g. evaluated using an EEG for recording heart rate or EKG for recording heart rhythm), a visual test, an auditory test, a biological sample (e.g. for evaluating changes in serum, plasma, whole blood, urine, sweat or the like), patient reporting (e.g. through questionnaires or surveys to evaluate mood, affect, coping, sleep quality, stress, anxiety, memory, and/or other emotions), functional data (e.g. evaluated by brain imaging (e.g. fNRIS, PET) or monitoring of brain activity levels (e.g. EEG)), body temperature, food intake, metabolic rate, perspiration, hydration, salivation, pupil dilation, breathing rate, pulse rate, skin colour, or skin temperature, and the like.

In some embodiments, the state of the individual can be measured or identified by pupil dilation (e.g., pupillometry), such as described in Example 2. In some embodiments, pupil dilation, such as measured by pupillometry, changes in an individual by at least 5% (e.g., at least 7%, at least 10%, at least 15%, at least 20%, at least 30%, at least 40%, at least 50%) after treatment with an active agent (e.g., such as 1 hour, 2 hours after treatment or 4 hours after treatment) as compared to baseline. In some embodiments, pupil dilation, such as measured by pupillometry, changes in an individual by at most 70% (e.g., at most 60%, at most 50%, at most 40%, at most 25%, at most 20%, at most 15%, at most 10%, at most 8%, at most 5%) after treatment with an active agent (e.g., such as 1 hour, 2 hours after treatment or 4 hours after treatment) as compared to baseline. In some embodiments, pupil dilation, such as measured by pupillometry, changes in an individual by at about 20% to about 30% after treatment with an active agent (e.g., such as 1 hour, 2 hours after treatment or 4 hours after treatment) as compared to baseline. In some embodiments, pupil dilation does not change in an individual after treatment as compared to baseline.

In some embodiments, the state of the individual can be measured or identified by reaction time and/or movement time (e.g., 5-choice reaction time task), such as described in Example 2. In some instances, such as described in Example 2 (FIG. 15), there may be a correlation between a measured state of an individual (e.g. change in pupil dilation) and RTI (e.g. reaction time and/or movement time). In some embodiments, an increase in pupil dilation (e.g. 1 hr increase in pupil dilation from baseline) is indicative of long term improvement in movement time (e.g. 4 hr change in RTI from baseline), indicative of the drug responsiveness of an individual.

In some embodiments, the state of the individual can be measured or identified by a questionnaire (e.g., STAI), such as described in Example 2. In some embodiments, the state of the individual can be measured or identified by reaction and/or movement time (e.g., 5-choice reaction time task), such as described in Example 2. In some embodiments, the state of the individual can be measured or identified by a questionnaire (e.g., STAI) and/or reaction and/or movement time (e.g., 5-choice reaction time task), such as described in Example 2. In some instances, such as described in Example 2 (Table 1), there may be a correlation between an individual's state (e.g. anxiety as measured at baseline for example by STAI) and improved reaction time (e.g., at 2 hr and/or 4 hr) following dosing with an active agent. In some instances, such as described in Example 2 (Table 1), there may be a correlation between an individual's reaction (e.g., measured at baseline (e.g., relative to others, or relative to a standard, or relative to their own performance over time) and improved reaction time (e.g. at 2 hr and/or 4 hr) following dosing with an active agent. In some embodiments, the individual's anxiety score such as measured by STAI at baseline (e.g. high anxiety score (e.g., >50 on STAI-S and/or STAI-T), corresponds to an improvement in reaction time at 2 hr (e.g., such as an improvement of at least 0.5%, at least 1%, at least 2.5%, at least 3%, at least 3.5%) or 4 hr (e.g., such as an improvement of at least 0.5%, at least 1%, at least 2.5%, at least 3%, at least 3.5%, at least 4%, at least 4.5%, at least 5%, at least 5.5%, at least 6%, at least 6.5%, at least 7%) following dosing of an active agent as compared to baseline. In some embodiments, the individual's anxiety score such as measured by STAI at baseline (e.g. high anxiety score (e.g., >50 on STAI-S and/or STAI-T), corresponds to an improvement in reaction time at 2 hr (e.g., such as an improvement of at most 50%, at most 40%, at most 30%, at most 25%, at most 20%, at most 15%, at most 10%, at most 5%) or 4 hr (e.g., such as an improvement of at most 50%, at most 40%, at most 30%, at most 25%, at most 20%, at most 15%, at most 10%, at most 5%) following dosing of an active agent as compared to baseline. In some embodiments, the individual's reaction time, such as measured by 5-choice reaction time task at baseline, corresponds to an improvement in reaction time at 2 hr (e.g at least 0.5%, at least 1%, at least 2.5%, at least 3%, at least 3.5%, at least 4%, at least 4.5%, at least 5%, at least 5.5%, at least 6%, at least 6.5%, at least 7%) or 4 hr (e.g. at least 0.5%, at least 1%, at least 2.5%, at least 3%, at least 3.5%, at least 4%, at least 4.5%, at least 5%, at least 5.5%, at least 6%, at least 6.5%, at least 7%) following dosing of an active agent as compared to baseline. In some embodiments, the individual's reaction time, such as measured by 5-choice reaction time task at baseline, corresponds to an improvement in reaction time at 2 hr (e.g at most 50%, at most 40%, at most 30%, at most 25%, at most 20%, at most 15%, at most 10%, at most 5%) or 4 hr (e.g. at most 50%, at most 40%, at most 30%, at most 25%, at most 20%, at most 15%, at most 10%, at most 5%) following dosing of an active agent as compared to baseline. In some embodiments, the individual's reaction time and/or anxiety score at baseline, corresponds to an improvement in reaction time at 2 hr (e.g. at least 0.5%, at least 1%, at least 2.5%, at least 3%, at least 3.5%, at least 4%, at least 4.5%, at least 5%, at least 5.5%, at least 6%, at least 6.5%, at least 7%) following dosing of an active agent as compared to baseline. In some embodiments, the individual's reaction time and/or anxiety score at baseline, corresponds to an improvement in reaction time at 2 hr (e.g. at most 50%, at most 40%, at most 30%, at most 25%, at most 20%, at most 15%, at most 10%, at most 5%) or 4 hr (e.g. at most 50%, at most 40%, at most 30%, at most 25%, at most 20%, at most 15%, at most 10%, at most 5%) following dosing of an active agent as compared to baseline. In some embodiments, the individual's reaction time, such as measured by 5-choice reaction time task at baseline, or the individual's anxiety score, such as measured by STAI at baseline, is indicative of long-term improvement in reaction time (e.g. 2 hr and/or 4 hr change in RTI from baseline), indicative of the drug responsiveness of an individual.

In some embodiments, the state of the individual may be measured or identified by evaluating one or more of EEG, EKG, MEG, fNIRS, PET, transcranial functional ultrasound, visual stimuli (e.g. colour, shape, pattern, emotional face, video, flash, milli second or longer), auditory stimuli (e.g. matched paired, acoustic frequency, Hz, milli second or longer), ultrasound waves by a sensor e.g., a piezoelectric resonant material (e.g., a PZT, a CMUT, a PMUT), clinical effect (visual, auditory, body, time and space, cognition, drowsiness, confusion, impairment), standard/deviant waveforms, evoked power/potential, voltage (alpha bands, beta bands, gamma bands, delta bands, or theta bands), asynchronization, time-locked, magnetic, hemodynamic (flux, flow, velocity, oxygenation), MMN, ASSR, surveys or questionnaires (written or verbal) including 5D-ASC, Beck Depression, Coping, DASS42, Gad-7 Anxiety, PANAS-GEN, SF-36 QOL, STAI, STAI 6, brain imaging tests, heart rate, pulse rate, respiratory rate, blood pressure, cardiovascular activity, photodiode, skin conduction and impedance, biological samples such as serum, plasma, whole blood, urine, sweat or the like, a genetic marker or the like (e.g. a brain-derived neurotrophic factor gene such as Val 66 or similar), visual perception alteration, an auditory perception alteration, bodily perception alteration, a temporal perception alteration, or a spatial perception alteration, sleep quality, patient reported outcomes (e.g. electronic) or subjective mood, affect, coping, sleep quality, stress, anxiety, memory, and other emotional or functional data with brain imaging or brain activity, providing data (e.g., patterns, clusters, classifications, amplitudes, frequencies, or magnitude) patient evaluation (e.g., physicians, counselors, psychologist, or spiritual leader), data comprising visual representations of brain responses or brain activity data and/or survey or patient reported outcomes to help convey the effectiveness or lack thereof of a psychedelic therapy, training modules, machine learning algorithms and training sets, artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory), auditory brainstem responses (ABR), paired pulse inhibition (PPI; ie P50 auditory suppression; sensory gating), auditory mismatch negativity (MNN), and auditory steady state responses (ASSR), if EEG measures of auditory sensory evoked activity, cognitive control of attention and emotion, sub-thalamic and cortical levels, amplitudes of MMN potentials will be correlated to depression, anxiety, and mood survey data, Emotional Flanker Task, Erikson Flanker Task, Continuous Performance Test (CPT), Connor's CPT, oddball tasks, State Trait Anxiety Inventory is a self-report survey, sleep diaries, the symmetry and power of alpha, beta, delta, and gamma brainwave activity across prefrontal cortex brain regions, Medical Quality of Life Outcomes Study 36-Item Short Form Health Survey (SF-36) and Physical Component Summary (PCS) and Mental Component Summary (MCS) scores, physical functioning, role physical, role emotional, bodily pain, vitality, social functioning, mental health, and general health on Likert, salivary cytokines, salivary brain-derived neurotrophic factors, psychophysical testing, assessing congruent, incongruent, and neutral conditions, surveys assessing mood, depression, anxiety, and stress, Mood Circumplex, DSM-V, cognitive behavioral therapy, synergy score, serenity score, quality of life, Sleep diary and Mood Capture, questions about attention, social interactions, fatigue and mood, approach avoid metric, biometric of coping, HRV spot check, or camera.

In some embodiments, the methods of the present application include measuring and identifying a state of an individual for the purposes of identifying a biomarker indicative of responsiveness to drug treatment (e.g. low responder). In some embodiments, the biomarker is used to identify high and low responding individuals. In some embodiments, the biomarker is useful in segregating individuals into treatable and non-treatable patient groups. In some embodiments, the methods herein are useful to identify those individuals that would benefit most from treatment. In some embodiments, the methods herein are useful as predictive tools for personalized and medical treatment. In some embodiments, the methods provided herein are useful in selecting patients for clinical trials. In some embodiments, the methods provided herein are useful for assessing eligibility of insurance coverage for individuals seeking drug treatment.

In certain embodiments, provided herein is a method of assessing and treating an individual comprising:
a. identifying or measuring a biomarker of the individual by performing an evaluation on the individual;
b. assessing the biomarker; and
c. if the biomarker is indicative of drug responsiveness, administering to the individual an active agent (e.g. 5-HT receptor agonist (e.g., psilocybin)), or a pharmaceutically acceptable salt, solvate, metabolite, derivative, or prodrug thereof.

In some embodiments, a test dose of the active agent (e.g. an amount used to assist in measuring of a biomarker (e.g. pupil dilation)) is administered to an individual prior to any one of the methods provided herein, such as described in Example 2. In some embodiments, a test dose of the active agent (e.g. an amount used to assist in measuring of a biomarker (e.g. pupil dilation)) is administered to an individual prior to identifying or measuring a biomarker. In some embodiments, a test dose of the active agent (e.g. an amount used to assist in measuring of a biomarker (e.g. pupil dilation)) is administered to an individual prior to identifying or measuring a state. Provided in some embodiments herein are methods for treating an individual suffering from a brain disease, disorder or condition (e.g., a mental, a behavioral, a neuropsychiatric, or the like, condition))). In some embodiments, provided herein are methods for treating or reducing the incidence of an episode in an individual susceptible to or suffering from a brain disease, disorder or condition (e.g., a mental, a behavioral, a neuropsychiatric, or the like, condition) by administering a dose (e.g., a therapeutically effective dose) of one or more 5-HT receptor agonist (e.g., psilocybin) to the individual.

In some embodiments, the dose (e.g., therapeutically effective dose) of the active agent (e.g., one or more 5-HT receptor agonist (e.g., psilocybin)) comprises about 0.5 mg to about 5 mg of the active agent (e.g., one or more 5-HT receptor agonist (e.g., psilocybin)). In some embodiments, the dose (e.g., therapeutically effective dose) of the active agent (e.g., one or more 5-HT receptor agonist (e.g., psilocybin)) comprises about 3 mg of the active agent (e.g., one or more 5-HT receptor agonist (e.g., psilocybin)). In some embodiments, the dose (e.g., therapeutically effective dose) of the active agent (e.g., one or more 5-HT receptor agonist (e.g., psilocybin)) is administered to an individual once daily. In some embodiments, the dose (e.g., therapeutically effective dose) of the active agent (e.g., one or more 5-HT receptor agonist (e.g., psilocybin)) is administered to an individual once every 3 days. In some embodiments, the dose (e.g., therapeutically effective dose) of the active agent (e.g., one or more 5-HT receptor agonist (e.g., psilocybin)) is administered to an individual for at least 28 days. In some embodiments, the dose (e.g., therapeutically effective dose) of the active agent (e.g., one or more 5-HT receptor agonist (e.g., psilocybin)) is administered to an individual for 28 days or less. In some embodiments, the dose (e.g., therapeutically effective dose) of the active agent (e.g., one or more 5-HT receptor agonist (e.g., psilocybin)) is administered to an individual for at least 28 days.

In some embodiments, the active agent (e.g., one or more 5-HT receptor agonist (e.g., psilocybin)) is formulated for oral administration. In some embodiments, the active agent (e.g., one or more 5-HT receptor agonist (e.g., psilocybin)) is formulated as a solution at a concentration of about 0.05 mg/mL to about 1 mg/mL. In some embodiments, the active agent (e.g., one or more 5-HT receptor agonist (e.g., psilocybin)) is formulated as a solution at a concentration of about 0.5 mg/mL.

In some embodiments, the evaluation is performed by the individual (e.g., in or outside a healthcare setting (e.g., in a natural environment, such as, for example, at home, at work, or the like)). In some embodiments, the evaluation is performed by a heath care provider (e.g., doctor, nurse, clinician).

Provided in some embodiments herein is a method for treating or reducing the incidence of a brain disorder or condition, and/or symptoms thereof, in an individual (e.g., in need thereof).

Provided in some embodiments herein is a method for treating or reducing the incidence of a mental, a behavioral, or a neuropsychiatric condition, and/or symptoms thereof, in an individual (e.g., in need thereof).

Provided in some embodiments herein is a method for treating attention deficit hyperactivity disorder (ADHD) or attention deficit disorder (ADD) in an individual (e.g., in need thereof).

Provided in some embodiments herein is a method for treating mild cognitive impairment, dementia, or Alzheimer's disease in an individual (e.g., in need thereof).

Provided in some embodiments herein is a method for improving coping in an individual (e.g., in need thereof). In some embodiments, coping is psychological coping. In some embodiments, the method is for improving stress recovery rate in the individual. In some embodiments, the individual is suffering from or susceptible to stress.

Provided in some embodiments herein is a method for increasing motivation in an individual (e.g., in need thereof). In some embodiments, the individual is suffering from or susceptible to low motivation, anxiety, apathy, fear, phobia, constructive impulsivity, depression, or the like. In some embodiments, the low motivation is induced by stress, anxiety, or the like.

Provided in some embodiments herein is a method for treating addiction in an individual (e.g., in need thereof).

Provided in some embodiments herein is a method for treating brain inflammation (e.g., encephalitis) or brain fog in an individual (e.g., in need thereof). In some embodiments, the brain inflammation or brain fog is secondary to a concussion, traumatic brain injury (TBI) (e.g., mild TBI (mTBI)), or the like. In some embodiments, the brain inflammation or brain fog is secondary to a natural insult (e.g., injury, stroke, or the like). In some embodiments, the individual is suffering from or susceptible to brain inflammation (e.g., encephalitis) or brain fog.

In some embodiments, the evaluation is identified or measured by performing a brain evaluation on the individual. In some embodiments, the brain evaluation is an electroencephalogram (EEG), a magnetoencephalogram (MEG), functional near-infrared spectroscopy (fNIRS), Positron Emission Tomography (PET), or a transcranial functional ultrasound imaging. In some embodiments, the evaluation is an EEG.

In some embodiments, the evaluation (e.g. brain test) is performed using a wearable device (e.g brain imaging). In some embodiments, the evaluation is recorded to a local memory or over a network using a mobile device (e.g., through a mobile application). In some embodiments, the method comprises patient reported outcomes or subjective mood, affect, coping, sleep quality, stress, anxiety, memory, and other emotional or functional data. In some embodiments, the method further comprises providing data (e.g., patterns, clusters, classifications, amplitudes, frequencies, or magnitude) on a device (e.g., a phone, tablet, or computer) to the individual and/or a caregiver (e.g., physicians, counselors, psychologist, or spiritual leader), the data comprising visual representations of brain responses or brain activity data and/or survey or patient reported outcomes to help convey the effectiveness or lack thereof of a psychedelic therapy.

In some embodiments, the method comprises performing one or more heart test (e.g., to determine heart rate, pulse rate, or cardiovascular activity) on the individual (e.g., for at least 30 seconds) (e.g., the one or more heart test being measured using a separate electrode sensitive voltage or sensor (e.g., photodiode) sensing an optical measure from visible light or infrared light). In some embodiments, the method comprises performing one or more test to measure blood pressure or respiratory rate.

In some embodiments, the method comprises performing one or more test to measure skin conductance or impedance.

In some embodiments, the method comprises measurement of a biological sample taken from the individual. In some embodiments, the biological sample is serum, plasma, whole blood, urine, or the like. In some embodiments the biological sample contains a genetic marker or the like. In some embodiments the genetic marker is a brain-derived neurotrophic factor gene or the like. In some embodiments the genetic marker is Val 66 or similar.

In some embodiments, the method is for treating a mental, behavioral, or neuropsychiatric condition, or the symptoms thereof. In some embodiments, the method is for managing a mental, a behavioral, or a neuropsychiatric condition, or the symptoms thereof. In some embodiments, the method is for treating and managing a mental, behavioral, or neuropsychiatric condition, or the symptoms thereof. In some embodiments, the individual is suffering from or susceptible to the mental, a behavioral, or a neuropsychiatric condition. In certain instances, the symptoms of the mental, a behavioral, or a neuropsychiatric condition are physical, behavioral, emotional, mental, or a combination thereof.

In some embodiments, the mental, the behavioral, or the neuropsychiatric condition is any disease or disorder provided herein. In some embodiments, the mental, the behavioral, or the neuropsychiatric condition is a symptom (e.g., provided herein) of any disease or disorder provided herein. In some embodiments, the mental, the behavioral, or the neuropsychiatric condition is a chronic condition.

In some embodiments, the mental, the behavioral, or the neuropsychiatric condition is a Diagnostic and Statistical Manual of Mental Disorders (DSM-5) category disease or disorder). In some embodiments, the mental, the behavioral, or the neuropsychiatric condition is a non-DSM-5 category disease or disorder.

In some embodiments, the mental, the behavioral, or the neuropsychiatric condition is an attention (e.g., an attention deficit) or a cognitive (e.g., neurocognitive) disorder or condition. In some embodiments, the mental, the behavioral, or the neuropsychiatric condition is a neurocognitive disorder or condition. In some embodiments, the mental, the behavioral, or the neuropsychiatric condition is an attention deficit disorder or condition. In some embodiments, the cognitive condition is mild cognitive impairment, dementia, or Alzheimer's disease.

In some embodiments, the mental, the behavioral, or the neuropsychiatric condition is induced by stress and/or anxiety.

In some embodiments, the mental, the behavioral, or the neuropsychiatric condition is selected from the group consisting of addiction, anxiety, apathy, attention (e.g., the lack thereof), and depression (e.g., moderate depression). In some embodiments, the mental, the behavioral, or the neuropsychiatric condition is PTSD, constructive impulsivity, a phobia, a fear, or the like. In some embodiments, the mental, the behavioral, or the neuropsychiatric condition is major depressive disorder.

In some embodiments, the individual is susceptible to or suffering from a brain disorder or condition (e.g., a mental condition, a behavioral condition, a neuropsychiatric condition, a brain state or a lack thereof (e.g., coping, motivation, stress, depression or anxiety), encephalitis, or a brain dysfunction (e.g., cognitive decline or brain fog).

In some embodiments, the condition is attention deficit hyperactivity disorder (ADHD) or attention deficit disorder (ADD).

In some embodiments, the condition is mild cognitive impairment, dementia, or Alzheimer's disease.

In some embodiments, the condition is addiction.

In various embodiments, methods provided herein are suitable for treating any suitable disorder, such as a neurological condition, such as a neurological disorder, or symptoms thereof. In specific embodiments, the neurological condition is a neurocognitive disorder. In various embodiments, methods provided herein are suitable for treating any suitable disorder or symptoms thereof including, but not limited to, feelings of distress, futility, disempowerment, despair, helplessness, and the like. In some embodiments, the disorder causing the feelings of distress, futility, disempowerment, despair, helplessness, and the like is a demoralization disorder. In some embodiments, symptoms of the neurological condition are physical, behavioral, emotional, mental or a combination thereof. In some embodiments, the neurological condition is an addictive disorder (e.g., alcohol abuse, substance abuse, smoking, or obesity). In some embodiments, the neurological condition is an eating disorder or an auditory disorder. In some embodiments, the neurological condition is pain (e.g., chronic pain). In some embodiments, the neurological condition is depression, bipolar disorder, post-traumatic stress disorder (PTSD), panic disorder, phobia, schizophrenia, psychopathy, or antisocial personality disorder. In some embodiments, the neurological condition is an impulsive disorder. In some embodiments, the impulsive disorder is attention deficit hyperactivity disorder (ADHD), Tourette's syndrome or autism. In some embodiments, the neurological condition is a compulsive disorder (e.g., obsessive compulsive disorder (OCD), gambling, or aberrant sexual behavior). In some embodiments, the neurological condition is a personality disorder (e.g., conduct disorder, antisocial personality, or aggressive behavior).

In some embodiments, administration of an active agent to a subject in need thereof occurs several times per day. In some embodiments, administration to a subject in need thereof occurs no more frequently than once a day (e.g., no more frequently than once every other day, no more frequently than once every third day, no more frequently than twice a week, no more frequently than once a week, no more frequently than once every two weeks, or the like). In some embodiments, administration to a subject in need thereof occurs once a day, every alternate day, three times a week, twice a week, once a week, every other week, two weeks per month, three weeks per month, once a month, twice a month or three times per month. In specific embodiments, administration is about once a day. In other specific embodiments, administration is about every alternate day. In still other specific embodiments, administration is about once a week.

In various embodiments, administration continues for any suitable length of time, such as at least one day, at least one week, at least 1 month, 2 months, 3 months, 4 months, 5 months, 6 months, 7 months, 8 months, 9 months, 10 months, 11 months, 12 months, 18 months, 2 years, 3 years, or more.

Provided in some embodiments here is a method comprising measuring (e.g., on a network (e.g., WiFi, cloud, BLE, 3/4/5G)) one or more brain response using a node or a wearable brain imaging device (e.g., an EEG), wherein the patient listens to paired or sustained auditory stimuli (e.g., at home, at work, or at school), the auditory stimuli being delivered from an application on a (e.g., mobile) device (e.g., cellular phone, tablet, or the like).

Provided in some embodiments herein is a method of imaging brain activity (e.g., voltage) in an individual using a wearable device (e.g., EEG (e.g., multi-electrode EEG device)), the wearable device having two or more electrodes that make contact with the forehead of the individual for at least thirty seconds and up to twenty minutes.

Provided in some embodiments herein is a method for optically imaging brain activity in an individual (e.g., from a wearable device) to determine, the predicted effectiveness of a psychedelic treatment, wherein a brain signal is identified by transmission of visible light (400-680 nanometers) or infrared light (greater than or equal to 680 nanometers) across the skin into the brain to collect reflected photons by a sensor (e.g., a photodiode or CMOS).

Provided in some embodiments is a method of acoustically imaging brain activity in an individual (e.g., from a wearable device) to determine the predictive effectiveness of a psychedelic treatment, wherein a brain signal is identified by transmission of ultrasound (e.g., having an acoustic frequency of greater than or equal to one megahertz) (e.g., across the skin and skull) into the brain to collect reflected ultrasound waves by a sensor (e.g., a piezoelectric resonant material (e.g., a PZT, a CMUT, a PMUT).

In some embodiments, the method comprises using artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory) for determining the effectiveness of a treatment paradigm or a patient to undergo treatment with psychedelic substances (e.g. psilocybin).

Provided in some embodiments herein is a computer-implemented method for determining or predicting the effectiveness of a psychedelic treatment administered to an individual.

Provided in some embodiments herein is a computer-implemented method for determining or predicting the effectiveness of a 5-HT receptor agonist in an individual. In some embodiments, the method comprises emitting one or more (e.g., auditory or visual) to the individual. In some embodiments, the method comprises receiving, from a brain imaging device (e.g., an EEG), a brain response.

In some embodiments, the method comprises receiving, from the individual, an emotional data. In some embodiments, the emotional data comprises a mood rating, a sleep rating, a stress rating, an anxiety rating, a memory rating, or any combination thereof.

In some embodiments, two or more of any step provided herein are performed simultaneously.

In some embodiments, two or more of any step provided herein are performed sequentially.

In some embodiments, the outcome of the evaluation is transmitted by a mobile device (e.g. cellular phone, tablet). In some embodiments, the outcome is transmitted over a wireless network (e.g. WiFi, cloud, BLE, 3/4/5G). In some embodiments, the outcome is determined by a machine learning algorithm.

Provided in some embodiments herein is a computer-implemented system for determining the outcome.

In some embodiments, the system comprises a digital processing device. In some embodiments, the digital processing device comprises at least one processor. In some embodiments, the digital processing device comprises an operating system configured to perform executable instructions. In some embodiments, the digital processing device comprises a memory. In some embodiments, the digital processing device comprises a computer program including instructions executable by the digital processing device to create an application.

Provided in some embodiment herein is a non-transitory computer-readable storage media encoded with a computer program including instructions executable by a processor to create an application for the method.

Provided in some embodiment herein is a non-transitory computer-readable storage media encoded with a computer program including instructions executable by a processor to create an application for the method.

In some embodiments, the application is configured to direct a device (e.g., an auditory or visual device) to emit one or more (auditory or visual) stimulus to the individual. In some embodiments, the application is configured to receive, from a brain imaging device (e.g. EEG), a brain response. In some embodiments, the application is configured to receive, from the individual, an emotional data. In some embodiments, the emotional data comprises a mood rating, a sleep rating, a stress rating, an anxiety rating, a memory rating, or any combination thereof.

In some embodiments, the application is configured to simultaneously perform two or more of steps provided herein.

In some embodiments, the application is configured to sequentially perform two or more of steps provided herein.

In some embodiments, the emotional data comprises data obtain from one or more questionnaires regarding, for example, depression, anxiety, stress, coping, mood, sleep, and quality of life of the individual.

In some embodiments, the one or more questionnaires can include a Beck Depression Inventory (BDI), a Generalized Anxiety Disorder (GAD-7), a Depression Anxiety Stress Scale (DASS), a Brief-COPE, a Positive and Negative Affect Schedule (PANAS), a State Trait Anxiety Inventory (STAI) (e.g. STAI-S and/or STAI-T), a modified version of a Russel Mood Circumplex, a modified version of a NIH Sleep Diary, a 36 Item Short Form Health Survey (SF-36), a 5D Altered state of Consciousness Scale (5d-ASC), and/or a daily sleep diary questionnaire.

In some embodiments, the application is configured to perform receiving (e.g., at least once weekly for 5 or more weeks) an individual's responses to the one or more questionnaires.

In some embodiments, the application is configured to perform scoring the individual's responses to the one or more questionnaires.

In some embodiments, the application is configured to perform receiving resting state brain activity from the individual. In some embodiments, the resting state brain activity is measured for at least thirty seconds (e.g., at least once, and up to four times, daily). In some embodiments, identifying the therapeutically effective dose of the 5-HT receptor agonist is based at least in part on changes to the resting state brain activity.

In some embodiments, receiving, from a brain imaging device (e.g., EEG), a brain response, comprises receiving amplitude and spectral power of EEG potentials measured from frontal, temporal, and parietal EEG sites in response to the one or more auditory and/or visual stimulus. In some embodiments, the one or more auditory stimulus comprises one or more auditory tasks comprising P50 paired click auditory suppression, Mismatch Negativity (MMN), and/or Auditory Steady State Response (ASSR). In some embodiments, the one or more visual stimulus comprises one or more visual tasks comprising an Emotional Flanker Task and/or a Continuous Performance Test (CPT).

In some embodiments, the evaluation uses one or more statistical methods comprising Bayesian methods, Mixed Model Repeated Measures (MMRM), repeated measures ANOVA and ANCOVA, and regression analyses.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows characterization of psilocybin (0.025-0.1 mg/kg SC), on food responding made available under a progressive schedule of reinforcement. Data is presented for both drugs as total number of lever presses recorded during the test session (A,D), final break point (B,E), and total session duration (C,F). Data for each drug is presented both as all test subjects (psilocybin: N=72), and subjects characterised as “low responders” based on having the lowest tertile on lever presses/break point based on performance measured over 7 days prior to onset of drug testing (psilocybin: N=24). The hashed line is to highlight the level of the “low responders” subgroup following vehicle pretreatment. *P<0.05 vs. vehicle control (Dunnetts test following significant ANOVA).

FIG. 2A-C shows a correlational analysis of the low performance subgroup from the psilocybin progressive ratio experiment (N=24 rats). FIG. 2A shows a correlational analysis from the psilocybin progressive ratio experiment for rats administered with 0.1 mg/kg of psilocybin. FIG. 2B shows a correlational analysis from the psilocybin progressive ratio experiment for rats administered with 0.05 mg/kg of psilocybin. FIG. 2C shows a correlational analysis from the psilocybin progressive ratio experiment for rats administered with 0.025 mg/kg of psilocybin. The rats were ranked based on baseline breakpoint and the % change for each rat following psilocybin treatment is presented. At both the 0.05 mg/kg and 0.1 mg/kg SC doses of psilocybin there was a significant correlation with increasing rank associated with a decreasing % increase in either break point or number of lever press, i.e the greatest increase in food maintained responding was seen in the lowest baseline responders. At the lowest (0.025 mg/kg) dose of psilocybin there was no significant correlation between baseline rank and % change.

FIG. 3 shows an overview of the Study Design (Part A) for a 2-Part, Phase 2a, Randomized, Double-Blind Trial to Evaluate the Safety, Tolerability, and Efficacy of Psilocybin Oral Solution in Adults with Generalized Anxiety Disorder.

FIG. 4 shows an overview of the Study Design (Part B) for a 2-Part, Phase 2a, Randomized, Double-Blind Trial to Evaluate the Safety, Tolerability, and Efficacy of Psilocybin Oral Solution in Adults with Generalized Anxiety Disorder (Example 3—the Trial).

FIG. 5 shows a summary of blood volume requirements for various blood screening assessments used in the Trial.

FIG. 6 shows a cover page of the user guide for the Diagnostic Assessment Research Tool (DART) used in the Trial.

FIG. 7 shows a copy of the Montgomery-Asberg Depression Rating Scale (MADRS) used in the Trial.

FIG. 8 shows a copy of the Naranjo Adverse Drug Reaction Probability Scale used in the Trial.

FIG. 9 shows a copy of the Generalized Anxiety Disorder Scale (GAD-7) used in the Trial.

FIG. 10 shows a copy of the Hospital Anxiety and Depression Scale used in the Trial.

FIG. 11 shows a copy of the Bowdle Visual Analog Scale used in the Trial.

FIG. 12 shows a copy of the Quality of Life Enjoyment and Satisfaction Questionnaire-Short Form used in the Trial.

FIG. 13 shows a copy of the Cognitive Failures Questionnaire used in the Trial.

FIG. 14 shows a copy of a STAI Questionnaire.

FIG. 15 shows a 1 hr change in MPD from baseline vs. longer term improvement in movement time.

FIG. 16 shows a non-limiting example of a computing device; in this case, a device with one or more processors, memory, storage, and a network interface.

### DETAILED DESCRIPTION

Provided in some embodiments herein are methods of assessing an individual's responsiveness to a drug therapy prior to the administration of an active agent.

### Certain Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an”, and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter disclosed.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The terms “effective amount” or “pharmaceutically effective amount” or “therapeutically effective amount” refer to a nontoxic but sufficient amount of the agent to provide the desired biological, therapeutic, and/or prophylactic result. That result might be reduction and/or alleviation of the signs, symptoms, or causes of a disease, or any other desired alteration of a biological system. For example, an “effective amount” for therapeutic uses is the amount of a 5-HT receptor agonist or a pharmaceutically acceptable salt, solvate, metabolite, derivative, or prodrug thereof as disclosed herein per se or a composition comprising a 5-HT receptor agonist or a pharmaceutically acceptable salt, solvate, metabolite, derivative, or prodrug thereof as disclosed herein required to provide a clinically significant decrease in a disease. An appropriate effective amount in any individual case might be determined by one of ordinary skill in the art using routine experimentation.

The term “5-HT receptor agonist agent” refers to a 5-HT receptor agonist as a free base or a derivate or analog thereof. Included in the term are salts, solvates, metabolites, prodrugs, isomers, tautomers, isotopic derivatives, and the like, of a 5-HT receptor agonist. In some embodiments, the derivates, analogs, salts, solvates, metabolites, prodrugs, isomers, tautomers, isotopic derivatives, etc are pharmaceutically acceptable derivates, analogs, salts, solvates, metabolites, prodrugs, isomers, tautomers, isotopic derivatives of a 5-HT receptor agonist.

The term “pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, metaphosphoric acid, nitric acid, phosphoric acid, and sulfuric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base to provide a “pharmaceutically acceptable base addition salt”. In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes might produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

The term “treating” and its grammatical equivalents as used herein include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding the fact that the patient might still be afflicted with the underlying disorder. For prophylactic benefit, a method might be performed on, or a composition might be administered to a patient at risk of developing a disease, or to a patient reporting one or more of the physiological symptoms of such conditions, even though a diagnosis of the condition might not have been made.

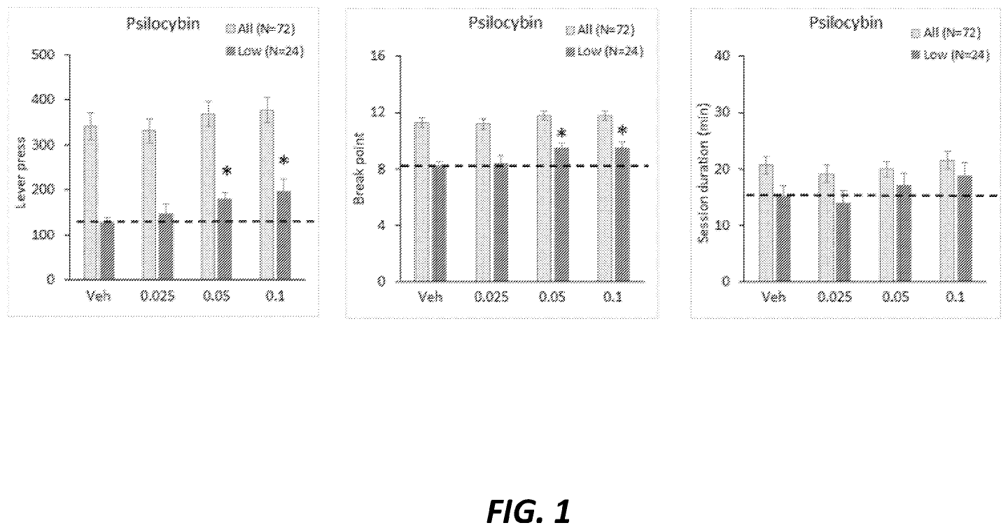

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, and tablet computers.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

As used in this specification and the appended claims, the terms “artificial intelligence,” “artificial intelligence techniques,” “artificial intelligence operation,” and “artificial intelligence algorithm” generally refer to any system or computational procedure that may take one or more actions that simulate human intelligence processes for enhancing or maximizing a chance of achieving a goal. The term “artificial intelligence” may include “generative modeling,” “machine learning” (ML), or “reinforcement learning” (RL).

As used in this specification and the appended claims, the terms “machine learning,” “machine learning techniques,” “machine learning operation,” and “machine learning model” generally refer to any system or analytical or statistical procedure that may progressively improve computer performance of a task. In some cases, ML may generally involve identifying and recognizing patterns in existing data in order to facilitate making predictions for subsequent data. ML may include a ML model (which may include, for example, a ML algorithm). Machine learning, whether analytical or statistical in nature, may provide deductive or abductive inference based on real or simulated data. The ML model may be a trained model. ML techniques may comprise one or more supervised, semi-supervised, self-supervised, or unsupervised ML techniques. For example, an ML model may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

Training the ML model may include, in some cases, selecting one or more untrained data models to train using a training data set. The selected untrained data models may include any type of untrained ML models for supervised, semi-supervised, self-supervised, or unsupervised machine learning. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some embodiments, the ML model may be stored in a database (e.g., associated with a server).

As described above, the machine learning model may implement a decision tree. A decision tree may be a supervised ML algorithm that can be applied to both regression and classification problems. Decision trees may mimic the decision-making process of a human brain. For example, a decision tree may grow from a root (base condition), and when it meets a condition (internal node/feature), it may split into multiple branches. The end of the branch that does not split anymore may be an outcome (leaf). A decision tree can be generated using a training data set according to the following operations: (1) Starting from a root node (the entire dataset), the algorithm may split the dataset in two branches using a decision rule or branching criterion; (2) each of these two branches may generate a new child node; (3) for each new child node, the branching process may be repeated until the dataset cannot be split any further; (4) each branching criterion may be chosen to maximize information gain (e.g., a quantification of how much a branching criterion reduces a quantification of how mixed the labels are in the children nodes). The labels may be the data or the classification that is predicted by the decision tree.

A random forest regression is an extension of the decision tree model that tends to yield more robust predictions by stretching the use of the training data partition. Whereas a decision tree may make a single pass through the data, a random forest regression may bootstrap 50% of the data (e.g., with replacement) and build many trees. Rather than using all explanatory variables as candidates for splitting, a random subset of candidate variables may be used for splitting, which may enable trees that have completely different data and different variables (hence the term random). The predictions from the trees, collectively referred to as the “forest,” may be then averaged together to produce the final prediction. Many trees (e.g., one hundred trees) may be included in a random forest model, with a number (e.g., 3, 6, 10, etc.) of terms sampled per split, a minimum of number (e.g., 1, 2, 4, 10, etc.) of splits per tree, and a minimum split size (e.g., 16, 32, 64, 128, 256, etc.). Random forests may be trained in a similar way as decision trees. Specifically, training a random forest may include the following operations: (1) select randomly k features from the total number of features; (2) create a decision tree from these k features using the same operations as for generating a decision tree; and (3) repeat the previous two operations until a target number of trees is created.

### 5-HT (or Serotonin) RECEPTORS

The 5-HT (or serotonin) receptors are a group of G protein-coupled receptors (GPCR) and ligand-gated ion channels. 5-HT is short for 5-hydroxy-tryptamine, the chemical name for serotonin.

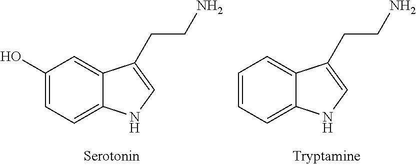 [SMILES: NCCc1c[nH]c2ccc(O)cc12 ; NCCc1c[nH]c2ccccc12]

The serotonin receptors are activated by serotonin, their natural ligand, and mediate both excitatory and inhibitory neurotransmission. They modulate the release of many neurotransmitters, including glutamate, GABA, dopamine, epinephrine/norepinephrine and acetylcholine, as well as many hormones, including oxytocin, prolactin, vasopressin, cortisol, corticotropin and substance P. The serotonin receptors influence various biological and neurological processes such as aggression, anxiety, appetite, cognition, learning, memory, mood, nausea, sleep, and thermoregulation.

The 5-HT receptors are divided into 7 families of G protein-coupled receptors. 5-HT₁, 5-HT₂, 5-HT₃ are the major families; the others, 5-HT₄, 5-HT₅, 5-HT₆ and 5-HT₇, for the most part, work in a similar fashion to either 5-HT₁ or 5-HT₂ receptors. The 5-HT receptors work with a G protein to modify an ion channel or membrane enzyme.

In certain instances, the 5-HT agonist of a formulation, composition, method, or the like described herein is a 5-HT₁ agonist. 5-HT₁ receptors have strong binding affinity for serotonin. Typically, when serotonin binds to a 5-HT₁ receptor, a G-protein is activated, opening an ion channel and allowing potassium ions to exit the neuron. This generally causes the neuron to become more negatively charged, making it more difficult to trigger an action potential, i.e. serotonin binding to 5-HT₁ receptors is an inhibitory effect.

In some preferred embodiments, the 5-HT agonist of a formulation, composition, method, or the like described herein is a 5-HT₂ agonist. In certain instances, the 5-HT₂ agonist has a relatively high affinity for 5-HT₂ receptors (e.g., relative to 5-HT₁ receptors and/or other 5-HT receptors, such as 5-HT₃, 5-HT₄, 5-HT₅, 5-HT₆, 5-HT₇, or all or some combination thereof, such as 2×, 3×, 5×, 10×, 20×, 50×, or the like greater affinity). 5-HT₂ receptors have weaker affinity for serotonin. As such, serotonin prefers to bind 5-HT₁ receptors, typically only binding 5-HT₂ receptors once the 5-HT₁ receptors are at least partially (or wholly) saturated. Serotonin binding of 5-HT₂ receptors typically activates a G-protein closing a potassium channel resulting in potassium ion build up. This generally causes depolarization, making it easier to reach the neuron's excitation threshold. Thus, when serotonin binds to 5-HT₂ receptors, it typically has an excitatory effect.

| Family | Type | Mechanism | Potential |
|---|---|---|---|
| 5-HT₁ | Protein coupled | Decreasing cellular levels of cAMP | Inhibitory |
| 5-HT₂ | Protein coupled | Increasing cellular levels of | Excitatory |
| | | IP₃ and DAG | |
| 5-HT₃ | Ligand-gated | Depolarizing plasma membrane | Excitatory |
| | Na⁺ and K⁺ | | |
| | cation channel | | |
| 5-HT₄ | Protein coupled | Increasing cellular levels of cAMP | Excitatory |
| 5-HT₅ | Protein coupled | Decreasing cellular levels of cAMP | Inhibitory |
| 5-HT₆ | Protein coupled | Increasing cellular levels of cAMP | Excitatory |
| 5-HT₇ | Protein coupled | Increasing cellular levels of cAMP | Excitatory |

The seven serotonin receptor families include fourteen receptor subtypes, distributed throughout the body as shown in the table below:

| | | Central | Peripheral | | | |
|---|---|---|---|---|---|---|
| 5HT | Blood | nervous | nervous | | | Smooth |
| Receptor | vessels | system | system | GI Tract | Platelets | Muscle |
| 1A | X | X | | | | |
| 1B | X | X | | | | |
| 1D | X | X | | | | |
| 1E | X | X | | | | |
| 1F | | X | | | | |
| 2A | X | X | X | X | X | X |
| 2B | X | X | X | X | X | X |
| 2C | X | X | X | X | X | X |
| 3 | | X | X | X | | |
| 4 | | X | X | X | | |
| 5A | | X | | | | |
| 5B | | | | | | |
| 6 | | X | | | | |
| 7 | X | X | | X | | |

### 5-HT₂ Receptors

In general, 5-HT₂ receptors are characterized by having lower affinity for serotonin (and other indolealkylamines), and are linked to the G_{q}/phospholipase C pathway of signal transduction. In various instances, such receptors mediate a variety of physiological and behavioral functions via three distinct subtypes: 5-HT_{2A}, 5-HT_{2B} and 5-HT_{2C}.

| Receptor | Physiological/behavioral function |
|---|---|
| 5-HT_{2A} | Addiction, Anxiety, Apathy, Appetite, Cognition, Depression, |
| | Imagination, Learning, Memory, Mood, Perception, Sexual |
| | Behavior, Sleep, Thermoregulation, Vasoconstriction |
| 5-HT_{2B} | Anxiety, Appetite, Cardiovascular Function, GI Motility, |
| | Sleep, Vasoconstriction |
| 5-HT_{2C} | Addiction, Anxiety, Appetite, GI Motility, Locomotion, |
| | Mood, Penile Erection, Sexual Behavior, Sleep, |
| | Thermoregulation, Vasoconstriction |

| Receptor | Uses of drugs that act on this receptor |
|---|---|
| 5-HT_{2A} | Antipsychotics, Psychedelics, Noradrenergic and Specific |
| | Serotonergic Antidepressants (NaSSAs), Sleeping aids |
| 5-HT_{2B} | Migraines |
| 5-HT_{2C} | Antidepressant, Orexigenic, Anorectic, Antipsychotic |

| Receptor | Agonists acting on receptor |
|---|---|
| 5-HT_{2A} | Bufotenin, Ergonovine, Lisuride, LSD, Mescaline, Myristicin, |
| | Psilocin, Psilocybin, DMT, DOM, PNU-22394, TFMPP, |
| | 25I-NBOMe, 2C-B, 5-MeO-DMT, BZP |
| 5-HT_{2B} | Fenfluramine, MDMA, Norfenfluramine, Methylphenidate |
| | 6-APB, BW-723C86, PNU-22394, Ro60-0175 |
| 5-HT_{2C} | Aripiprazole, Ergonovine, Lorcaserin, Trazodone |
| | PNU-22394, Ro60-0175, TFMPP, YM-348, A-372,159, |
| | AL-38022A |

5-HT_{2A} is an important excitatory serotonin receptor subtype. In some instances, physiological processes mediated by the receptor include, by way of non-limiting example:
central nervous system—neuronal excitation, behavioral effects, learning, anxiety, and pro-nociception
smooth muscle contraction (in bronchi and gastrointestinal tract)
vasoconstriction/vasodilation
platelet aggregation
role in memory and learning
anti-inflammatory activity
hormone (oxytocin, prolactin, ACTH, corticosterone, renin) regulation
mood regulation (depressed patients have more 5-HT_{2A} receptors than otherwise normal individuals implying 5-HT_{2A} is involved in the pathogenesis of depression)

In some instances, agonism of 5-HT_{2A} agonism facilitates treatment or management of disorders involving cognitive function and social interaction, or the symptoms thereof, as evidenced by the extensive localization of the 5-HT_{2A} receptor in brain areas that mediate cognitive functions and social interaction. In some instances, disorders in which the 5-HT_{2A} receptor are involved include, but are not limited to schizophrenia, apathy, depression/suicide (e.g., low motivation), anxiety, obsessive compulsive disorders (OCD), bipolar disorders, attention deficit hyperactivity disorder (ADHD), eating disorders such as anorexia nervosa, autism and autism spectrum disorders, Asperger's, neuropsychiatric diseases and disorders, sexual disorders such as erectile dysfunction, neurodegenerative diseases, inflammatory diseases, autoimmune diseases, metabolic diseases such as obesity and diabetes, central nervous system disorders, peripheral nervous system disorders, Alzheimer's disease, snoring, sleep apnea (obstructive sleep apnea, central sleep apnea), insomnia, sleep deprivation, restless legs syndrome, parasomnia, nightmares, night terrors, sleepwalking, hypersomnia (daytime sleepiness), narcolepsy and pain.

Any suitable 5-HT (e.g., 5-HT₂, such as 5-HT_{2A}) agonist is utilized in any composition, formulation, method, therapy, or the like described herein. In some preferred embodiments, the 5-HT agonist of a formulation, composition, method, or the like described herein is a 5-HT_{2A} agonist. In certain instances, the 5-HT_{2A} agonist has a relatively high affinity for 5-HT_{2A} receptors (e.g., relative to 5-HT₁, 5-HT₃, 5-HT₄, 5-HT₅, 5-HT₆, 5-HT₇, 5-HT_{2B}, 5-HT_{2C}, or all or some combination thereof, such as 2×, 3×, 5×, 10×, 20×, 50×, or the like greater affinity). In some instances, 5-HT_{2A} agonists increase dopamine levels in the prefrontal cortex. In certain instances, the 5-HT_{2A} agonist provided herein is one of the following classes of 5-HT_{2A} agonists: the ergolines, tryptamines and phenethylamines.

### Ergolines

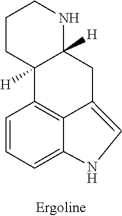 [SMILES: c1cc2c3c(c[nH]c3c1)C[C@H]1NCCC[C@H]21]

In specific embodiments, a 5-HT (e.g., 5-HT_{2A}) receptor agonist utilized herein is an ergoline. In some instances, ergonovine and ergotamine, synthetic derivatives include the oxytocic methergine, the anti-migraine drugs dihydroergotamine and methysergide, hydergine (a mixture of dihydroergotoxine mesylates, INN: ergoline mesylates), and bromocriptine. In certain instances, synthetic ergolines include pergolide and lisuride.

In certain instances, the ergoline is an ergoline derivative, such as a lysergic acid amide or a peptide alkaloid, such as described below. In some instances, the ergoline is a clavine (examples include festuclavine, fumigaclavine A, fumigaclavine B and fumigaclavine C) and other derivatives that do not fall into these categories, such as cabergoline, pergolide, lisuride.

### Lysergic Acid Amides

Exemplary lysergic acid amides include Ergine (LSA, D-lysergic acid amide), Ergonovine (ergobasine), Methergine (ME-277), Methysergide (UML-491), LSD (D-lysergic acid diethylamide), LSH (D-lysergic acid α-hydroxyethylamide). The table below summarizes their structural formula and relationships.

| | Name | R¹ | R² | R³ |
|---|---|---|---|---|
| 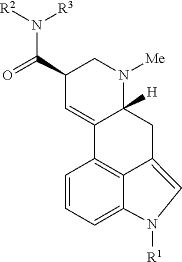 | Ergine Ergonovine Methergine Methysergide LSD LSH | H H H CH₃ H H | H CH(CH₃)CH₂OH CH(CH₂CH₃)CH₂OH CH(CH₂CH₃)CH₂OH CH₂CH₃ CH(OH)CH₃ | H H H H CH₂CH₃ H |

### Peptide Alkaloids

Exemplary peptide alkaloids include, peptide ergot alkaloids (ergopeptines or ergopeptides), which are ergoline derivatives containing a tripeptide structure (attached at the same position as the amide group of the lysergic acid derivatives) comprising proline and two other α-amino acids. Examples include:
Ergotoxines (valine at R²)—Ergocristine, Ergocornine, α-Ergocryptine, β-Ergocryptine
Ergotamines (alanine at R²)—Ergotamine, Ergovaline, α-Ergosine, β-Ergosine.

| | | | | | R³ Amino |
|---|---|---|---|---|---|
| | Name | R¹ | R² | R³ | acid |
| 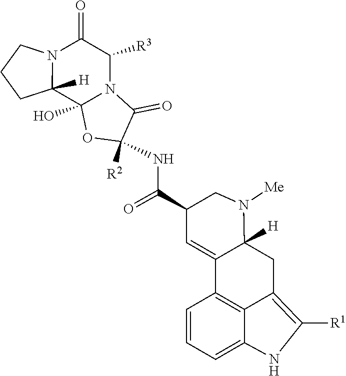 | Ergocristine Ergocornine α-Ergocryptine β-Ergocryptine Ergotamine Ergovaline α-Ergosine β-Ergosine Bromocriptine | Br | CH(CH₃)₂ CH(CH₃)₂ CH(CH₃)₂ CH(CH₃)₂ CH₃ CH₃ CH₃ CH₃ CH(CH₃)₂ | benzyl CH(CH₃)₂ CH₂CH(CH₃)₂ (S)-CH(CH₃)CH₂CH₃ benzyl CH(CH₃)₂ CH₂CH(CH₃)₂ (S)-CH(CH₃)CH₂CH₃ CH₂CH(CH₃)₂ | Phenylalanine Valine Leucine Isoleucine Phenylalanin Valine Leucine Isoleucine Leucine |

### Tryptamines

Tryptamine (2-(1H-Indol-3-yl)ethanamine) comprises an indole ring, attached to an aminoethylene group; substituted tryptamines are substituted with any suitable group, such as being modified on the indole ring (R¹, R²), the ethylene chain (R³) and/or on the amino group (R⁴, R⁵), as illustrated below, and are collectively referred to herein as tryptamines. Examples of tryptamines include serotonin, melatonin, psilocybin and N,N-Dimethyltryptamine. Additionally, the tryptamine structure may comprise part of a more complex compound, for example: LSD, ibogaine, mitragynine, yohimbine, etc.

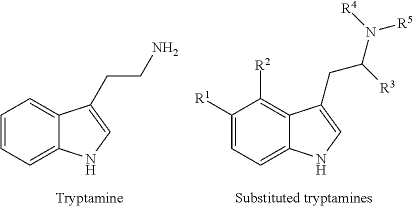

Examples of naturally occurring substituted tryptamines include, by way of non-limiting example:

| Short/Common | | | | | | |
|---|---|---|---|---|---|---|
| Name | Full Name | R¹ | R² | R³ | R⁴ | R⁵ |
| Tryptamine | 3-(2-aminoethyl)indole | H | H | H | H | H |
| | 2-(1H-indol-3- | | | | | |
| | yl)ethanamine | | | | | |
| Bufotenin | 5-hydroxy-N,N- | OH | H | H | CH₃ | CH₃ |
| | dimethyltryptamine | | | | | |
| Nω- | 5-hydroxy-N- | OH | H | H | CH₃ | H |
| Methylserotonin | methyltryptamine | | | | | |
| (norbufotenin) | | | | | | |
| Serotonin | 5-hydroxytryptamine | OH | H | H | H | H |
| NMT | N-methyltryptamine | H | H | H | H | CH₃ |
| 5-MeO-NMT | 5-methoxy-N- | OCH₃ | H | H | CH₃ | H |
| | methyltryptamine | | | | | |
| DMT | N,N-dimethyltryptamine | H | H | H | CH₃ | CH₃ |
| 5-Bromo- | 5-bromo-N,N- | Br | H | H | CH₃ | CH₃ |
| DMT | dimethyltryptamine | | | | | |
| 5-MeO-DMT | 5-methoxy-N,N- | OCH₃ | H | H | CH₃ | CH₃ |
| | dimethyltryptamine | | | | | |
| Melatonin | 5-methoxy-N- | OCH₃ | H | H | C(O)CH₃ | H |
| | acetyltryptamine | | | | | |
| N- | 5-hydroxy-N- | OH | H | H | C(O)CH₃ | H |
| Acetylserotonin | acetyltryptamine | | | | | |
| Norbaeocystin | 4-phosphoryloxy- | H | OPO₃H₂ | H | H | H |
| | tryptamine | | | | | |
| Baeocystin | 4-phosphoryloxy-N- | H | OPO₃H₂ | H | CH₃ | H |
| | methyl-tryptamine | | | | | |
| Psilocybin | 4-phosphoryloxy-N,N- | H | PO₄ | H | CH₃ | CH₃ |
| | dimethyltryptamine | | | | | |
| Psilocin | 4-hydroxy-N,N- | H | OH | H | CH₃ | CH₃ |
| | dimethyltryptamine | | | | | |
| Tryptophan | α-carboxyltryptamine | H | H | COOH | H | H |

Examples of synthetic substituted tryptamines include, by way of non-limiting example:

| Short | | | | | | |
|---|---|---|---|---|---|---|
| Name | Name | R¹ | R² | R³ | R⁴ | R⁵ |
| αET | α-ethyltryptamine | H | H | CH₂ | H | H |
| | | | | CH₃ | | |
| αMT | α-methyltryptamine | H | H | CH₃ | H | H |
| DALT | N,N-diallyltryptamine | H | H | H | H₂C═CH—CH₂ | H₂C═CH—CH₂ |
| DET | N,N-diethyltryptamine | H | H | H | CH₂CH₃ | CH₂CH₃ |
| DiPT | N,N- | H | H | H | CH(CH₃)₂ | CH(CH₃)₂ |
| | diisopropyltryptamine | | | | | |
| DPT | N,N-dipropyltryptamine | H | H | H | CH₂CH₂CH₃ | CH₂CH₂CH₃ |
| 5-MeO- | 5-methoxy-α- | OCH₃ | H | CH₃ | H | H |
| αMT | methyltryptamine | | | | | |
| 5-MeO- | 5-methoxy-N,N- | OCH₃ | H | H | H₂C═CH—CH₂ | H₂C—CH—CH₂ |
| DALT | diallyltryptamine | | | | | |
| 5-MeO- | 5-methoxy-N-Methyl-N- | OCH₃ | H | H | H₂C═CH—CH₂ | CH₃ |
| MALT | allyltryptamine | | | | | |
| 4-HO- | 4-hydroxy-N,N- | H | OH | H | CH₂CH₃ | CH₂CH₃ |
| DET | diethyltryptamine | | | | | |
| 4-AcO- | 4-acetoxy-N,N- | H | OCOCH₃ | H | CH₃ | CH₃ |
| DMT | dimethyltryptamine | | | | | |
| 4-HO- | 4-hydroxy-N-methyl-N- | H | OH | H | CH₃ | CH₂CH₃ |
| MET | ethyltryptamine | | | | | |
| 4-HO- | 4-hydroxy-N,N- | H | OH | H | CH(CH₃)₂ | CH(CH₃)₂ |
| DIPT | diisopropyltryptamine | | | | | |
| 5-MeO- | 5-methoxy-N,N- | OCH₃ | H | H | CH(CH₃)₂ | CH(CH₃)₂ |
| DIPT | diisopropyltryptamine | | | | | |
| 5-MeO- | 5-methoxy-N,N- | OCH₃ | H | H | CH₃ | CH(CH₃)₂ |
| MiPT | methylisopropyltryptamine | | | | | |
| 4-HO- | 4-hydroxy-N-isopropyl- | H | OH | H | CH(CH₃)₂ | CH₃ |
| MiPT | N-methyltryptamine | | | | | |
| Sumatriptan | 5-(methylamino | CH₂SO₂NH | H | H | CH₃ | CH₃ |
| | sulfonylmethylene)-N,N- | CH₃ | | | | |
| | dimethyltryptamine | | | | | |
| Zolmitriptan | 5-(4-(S)-1,3-oxazolidin- | CHNHC(O) | H | H | CH₃ | CH₃ |
| | 2-one)-N,N-dimethyltryptamine | OCH₂ | | | | |

### Phenethylamines

Phenethylamine comprises a phenyl ring attached to an aminoethylene group; substituted phenethylamines are optionally substituted in any suitable manner, such as they are optionally modified by substitution on the phenyl ring (R¹, R², R³, R⁴ and/or R⁵), the ethylene chain (R⁶ and/or R⁷) and/or on the amino group (R⁸, and/or R⁹), such as illustrated below.

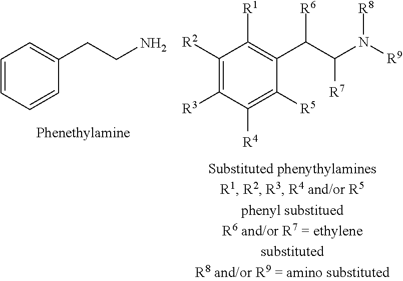

Examples of phenethylamines include, but are not limited those presented in the table below:

| | | Substitution | | | Biological |
|---|---|---|---|---|---|
| Short Name | Full Name | Amino | Ethylene | Phenyl | activity |
| Amphetamine | α- | | X | | Stimulant |
| | methylphenethylamine | | | | |
| β-Methyl | β- | | X | | Stimulant |
| phenethylamine | methylphenethylamine | | | | |
| Mephedrone | 4-methylmethcathinone | X | X | X | Stimulant |
| Ethcathinone | N-ethylcathinone | X | X | | Stimulant |
| Ephedrine/ | N-methyl-β- | X | X | | Stimulant; |
| Pseudoephedrine | hydroxyamphetamine | | | | decongestant |
| Methamphetamine | N-methylamphetamine | X | X | | Stimulant; |
| | | | | | neurotoxin |
| Phentermine | α-methylamphetamine | | X | | Stimulant, |
| | | | | | anorectic |
| Ortetamine | 2-methylamphetamine | | X | X | Stimulant, |
| | | | | | anorectic |
| Amfepramone | N-diethyl-β- | X | X | | Anorectic |
| (diethylpropion) | ketoamphetamine | | | | |
| Phenylephrine | β,3-dihydroxy-N- | X | X | X | Decongestant |
| | methylphenethylamine | | | | |
| Methylphenidate | N,α-butylene-β-methoxy | X | X | | Stimulant; NDRI |
| | carbonylphenethylamine | | | | |
| Dopamine | 3,4- | | | X | Catecholamine |
| | dihydroxyphenethylamine | | | | neurotransmitter |
| 6- | 2,4,5-trihydroxy | | | X | Neurotoxic |
| Hydroxydopamine | phenethylamine | | | | agent |
| Epinephrine | β-3,4-trihydroxy-N- | X | X | X | Catecholamine |
| (Adrenaline) | methylphenethylamine | | | | neurotransmitter |
| Norepinephrine | β-3,4-trihydroxy | | X | X | Catecholamine |
| (Noradrenaline) | phenethylamine | | | | neurotransmitter |
| para-Octopamine | β-4-dihydroxy | | X | X | Trace aminergic |
| | phenethylamine | | | | α-adrenoceptor |
| | | | | | agonist |
| Salbutamol | β-4-dihydroxy-3- | X | X | X | Short-action β2- |
| | hydroxymethyl-N-tert- | | | | adrenergic |
| | butylphenethylamine | | | | agonist |
| N-Methyl | N- | X | | | Amphetamine |
| phenethylamine | methylphenethylamine | | | | isomer |
| Cathine | d-β- | | X | | Releasing agent |
| | hydroxyamphetamine | | | | |
| Cathinone | β-ketoamphetamine | | X | | Releasing agent |
| Methcathinone | N-methylcathinone | X | X | | Releasing agent |
| Bupropion | 3-chloro-N-tert-butyl-β- | X | X | X | NDRI |
| | ketoamphetamine | | | | |
| Norfenfluramine | 3-trifluoromethyl- | | X | X | SSRA |
| | amphetamine | | | | |
| Fenfluramine | 3-trifluoromethyl-N- | X | X | X | SSRA |
| | ethylamphetamine | | | | |
| Mescaline | 3,4,5-trimethoxy | | | X | Psychedelic |
| | phenethylamine | | | | |
| Proscaline | 2-(3,5-dimethoxy-4- | | | X | Psychedelic |
| | propoxyphenyl)ethanamine | | | | |
| Metaescaline | 2-(3-ethoxy-4,5- | | | X | Psychedelic |
| | dimethoxyphenyl)ethanamine | | | | |
| Allylescaline | 4-Allyloxy-3,5- | | | X | Psychedelic |
| | dimethyloxy | | | | |
| | phenylethylamine | | | | |
| Methallylescaline | 4-Methallyloxy-3,5- | | | X | Psychedelic |
| | dimethoxyphenethylamine | | | | |
| Asymbescaline | 3,4-Diethoxy-5- | | | X | Psychedelic |
| | methoxyphenethylamine | | | | |

In some embodiments, the method comprises administering an amount of one or more 5-HT receptor agonist (e.g., psilocybin), or a pharmaceutically acceptable salt, solvate, metabolite, derivative, or prodrug thereof. In some embodiments, the method comprises administering an amount of an anti-depressant, or a pharmaceutically acceptable salt thereof. In some embodiments, the method comprises administering an amount of one or more 5-HT receptor agonist (e.g., psilocybin), or a pharmaceutically acceptable salt, solvate, metabolite, derivative, or prodrug thereof and an amount of an anti-depressant, or a pharmaceutically acceptable salt thereof.

In some embodiments, the amount of the one or more 5-HT receptor agonist is a therapeutically effective amount of the one or more 5-HT receptor agonist. In some embodiments, the amount of the anti-depressant is a therapeutically effective amount of the anti-depressant. Non-limiting examples of anti-depressants contemplated for use herein include, for example, SSRIs, tricyclic antidepressants (TCAs), serotonin and norepinephrine reuptake inhibitors (SNRIs), norepinephrine and dopamine reuptake inhibitors (NDRIs), monoamine oxidase inhibitors (MAOIs), serotonin-norepinephrine reuptake inhibitors (SNRIs), serotonin modulator and stimulators (SMSs), serotonin antagonists and reuptake inhibitors (SARIs), norepinephrine reuptake inhibitors (NRIs), tetracyclic antidepressants, NMDA receptor antagonists and atypical antidepressants.

In some embodiments, the anti-depressant is selected from the group consisting of Abilify (aripiprazole), Adapin (doxepin), Anafranil (clomipramine), Aplenzin (bupropion), Asendin (amoxapine), Aventyl HCI (nortriptyline), Celexa (citalopram), Cymbalta (duloxetine), Desyrel (trazodone), Effexor XR (venlafaxine), Emsam (selegiline), Etrafon (perphenazine and amitriptyline), Elavil (amitriptyline), Endep (amitriptyline), Fetzima (levomilnacipran), Khedezla (desvenlafaxine), Latuda (lurasidone), Lamictal (lamotrigine), Lexapro (escitalopram), Limbitrol (amitriptyline and chlordiazepoxide), Luvox (fluvoxamine), Marplan (isocarboxazid), Nardil (phenelzine), Norpramin (desipramine), Oleptro (trazodone), Pamelor (nortriptyline), Parnate (tranylcypromine), Paxil (paroxetine), Pexeva (paroxetine), Prozac (fluoxetine), Pristiq (desvenlafaxine), Remeron (mirtazapine), Sarafem (fluoxetine), Seroquel XR (quetiapine), Serzone (nefazodone), Sinequan (doxepin), Surmontil (trimipramine), Symbyax (fluoxetine and the atypical antipsychotic drug olanzapine), Tofranil (imipramine), Triavil (perphenazine and amitriptyline), Trintelllix (vortioxetine), Viibryd (vilazodone), Vivactil (protriptyline), Wellbutrin (bupropion), Zoloft (sertraline), and Zyprexa (olanzapine).

In some embodiments, described herein are pharmaceutical compositions, comprising 5-HT receptor agonist, or pharmaceutically acceptable salt, solvate, metabolite, derivative, or prodrug thereof, such agents being collectively referred to herein as 5-HT receptor agonist agents and/or an antidepressant. In some instances, the pharmaceutical formulations of 5-HT receptor agonist, or pharmaceutically acceptable salt, solvate, metabolite, derivative, or prodrug thereof, have enhanced bioavailability and efficacy, have a lower administration dose, a lower cytotoxicity, and/or have decreased side effects.

In various embodiments provided herein, any suitable route of administration is contemplated. In specific embodiments, the composition is formulated for oral, buccal, nasal or inhalation administration. In some embodiments, the composition is an oral, buccal, nasal or inhalation composition.

In specific embodiments, the composition further comprises any suitable (e.g., pharmaceutically acceptable) excipients and/or additives, such as surfactants, preservatives, flavoring agents, sweetening agents, or anti-foaming agents.

Any suitable composition, formulation, or dosage form is contemplated herein. In some embodiments, the composition, formulation, or dosage form is an oral composition, formulation or dosage form. In some specific embodiments, the pharmaceutically acceptable excipient is selected from the group consisting of fillers, binders, suspending agents, disintegrants, lubricants, and combinations thereof.

Provided herein are methods for managing disorders or conditions, comprising administering one or more 5-HT receptor agonists, or pharmaceutically acceptable salts, solvates, metabolites, derivatives, or prodrugs thereof.

Further provided herein are methods for treating symptoms of disorders or conditions, comprising administering one or more 5-HT receptor agonists, or pharmaceutically acceptable salts, solvates, metabolites, derivatives, or prodrugs thereof.

Further provided herein is a method of treating any disorder or condition treatable by 5-HT receptor agonism.

Provided in some embodiments herein is a method for treating or reducing the incidence of a brain disorder, comprising administering one or more 5-HT receptor agonists, or pharmaceutically acceptable salts, solvates, metabolites, derivatives, or prodrugs thereof.

Provided in some embodiments herein is a method for identifying a biomarker in an individual prior to administration of a drug. In some embodiments, the method comprises identifying or measuring a state of an individual by performing an evaluation (e.g., pre-dosing) on the individual. In some embodiments, the effectiveness of drug therapy (e.g. the active agent) is determined (at least in part) by the identification or measurement of the evaluation. In some embodiments, the method comprises: 1) identifying or measuring the state of the individual by performing an evaluation (e.g., pre-dosing) on the individual, and 2) administering an active agent based on the identification or measurement of the evaluation.

In certain embodiments, the state of an individual can be a physiological, neurological, psychological, metabolic, or biological state or a combination thereof. In some embodiments the state of an individual is measured or identified by a brain activity (e.g. using a brain evaluation (e.g. EEG, MEG, fNRIS, PET transcranial functional ultrasound)), a heart test (e.g. evaluated using an EEG for recording heart rate or EKG for recording heart rhythm), a visual test, an auditory test, a biological sample (e.g. for evaluating changes in serum, plasma, whole blood, urine, sweat or the like), patient reporting (e.g. through questionnaires or surveys to evaluate mood, affect, coping, sleep quality, stress, anxiety, memory, and/or other emotions), functional data (e.g. evaluated by brain imaging (e.g. fNRIS, PET) or monitoring of brain activity levels (e.g. EEG)), body temperature, food intake, metabolic rate, perspiration, hydration, salivation, pupil dilation, breathing rate, pulse rate, skin colour, or skin temperature, and the like.

In some embodiments, the state of the individual may be measured or identified by evaluating one or more of EEG, EKG, MEG, fNIRS, PET, transcranial functional ultrasound, visual stimuli (e.g. colour, shape, pattern, emotional face, video, flash, milli second or longer), auditory stimuli (e.g. matched paired, acoustic frequency, Hz, milli second or longer), ultrasound waves by a sensor e.g., a piezoelectric resonant material (e.g., a PZT, a CMUT, a PMUT), clinical effect (visual, auditory, body, time and space, cognition, drowsiness, confusion, impairment), standard/deviant waveforms, evoked power/potential, voltage (alpha bands, beta bands, gamma bands, delta bands, or theta bands), asynchronization, time-locked, magnetic, hemodynamic (flux, flow, velocity, oxygenation), MMN, ASSR, surveys including 5D-ASC, Beck Depression, Coping, DASS42, Gad-7 Anxiety, PANAS-GEN, SF-36 QOL, STAI, STAI 6, brain imaging tests, heart rate, pulse rate, respiratory rate, blood pressure, cardiovascular activity, photodiode, skin conduction and impedance, biological samples such as serum, plasma, whole blood, urine, sweat or the like, a genetic marker or the like (e.g. a brain-derived neurotrophic factor gene such as Val 66 or similar), visual perception alteration, an auditory perception alteration, bodily perception alteration, a temporal perception alteration, or a spatial perception alteration, sleep quality, patient reported outcomes (e.g. electronic) or subjective mood, affect, coping, sleep quality, stress, anxiety, memory, and other emotional or functional data with brain imaging or brain activity, providing data (e.g., patterns, clusters, classifications, amplitudes, frequencies, or magnitude) patient evaluation (e.g., physicians, counselors, psychologist, or spiritual leader), data comprising visual representations of brain responses or brain activity data and/or survey or patient reported outcomes to help convey the effectiveness or lack thereof of a psychedelic therapy, training modules, machine learning algorithms and training sets, artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory), auditory brainstem responses (ABR), paired pulse inhibition (PPI; ie P50 auditory suppression; sensory gating), auditory mismatch negativity (MNN), and auditory steady state responses (ASSR), if EEG measures of auditory sensory evoked activity, cognitive control of attention and emotion, sub-thalamic and cortical levels, amplitudes of MMN potentials will be correlated to depression, anxiety, and mood survey data, Emotional Flanker Task, Erikson Flanker Task, Continuous Performance Test (CPT), Connor's CPT, oddball tasks, State Trait Anxiety Inventory is a self-report survey, sleep diaries, the symmetry and power of alpha, beta, delta, and gamma brainwave activity across prefrontal cortex brain regions, Medical Quality of Life Outcomes Study 36-Item Short Form Health Survey (SF-36) and Physical Component Summary (PCS) and Mental Component Summary (MCS) scores, physical functioning, role physical, role emotional, bodily pain, vitality, social functioning, mental health, and general health on Likert, salivary cytokines, salivary brain-derived neurotrophic factors, psychophysical testing, assessing congruent, incongruent, and neutral conditions, surveys assessing mood, depression, anxiety, and stress, Mood Circumplex, DSM-V, cognitive behavioral therapy, synergy score, serenity score, quality of life, Sleep diary and Mood Capture, questions about attention, social interactions, fatigue and mood, approach avoid metric, biometric of coping, HRV spot check, or camera.

In some embodiments, the state of the individual may be measured or identified by administering a 5-choice reaction time task (RTI). In some instances, the RTI comprises subjects holding down a button at the bottom of the screen until a yellow spot appears in 1 of 5 circles at the top of the screen. In some instances, the subject then releases the button and touch inside the circle where the yellow spot appeared as quickly as they can. In some embodiments, key outcomes of RTI are median 5-choice reaction time and movement time. In some embodiments, the state of the individual may be measured or identified by administering a RTI at baseline (e.g., before administration of an active agent). In some embodiments, the state of the individual may be measured or identified by administering a RTI 2 hours post dosing. In some embodiments, the state of the individual may be measured or identified by administering a RTI 4 hours post dosing. In some embodiments, the median five-choice reaction time or median time (e.g., such as at 2 hours or 4 hours) after treatment is compared to baseline (e.g., to assess percent change after treatment), such as described in Example 2. In some embodiments, the median five-choice reaction time or median time can be used to identify “low-responders” who may respond to treatment with an active agent provided herein.

In some embodiments, identifying or measuring a state is performed through measurement of pupil dilation in an individual. In some embodiments, the state of the individual may be measured or identified by pupillometry, such as measuring pupil dilation using a pupilometer. In some embodiments, pupil size is measured before administration of an active agent provided herein. In some embodiments, pupil size is measured after administration (e.g., such as 1 hour, 2 hours or 4 hours after administration) of an active agent provided herein. In some embodiments, baseline pupil size (e.g., before administration of the active agent) is compared to pupil size after treatment (e.g., such as 1 hour, 2 hours or 4 hours after treatment), such as described in Example 2. In some instances, such as described in Example 2, pupil dilation may be used to identify “low-responders” who may respond to treatment with an active agent provided herein.

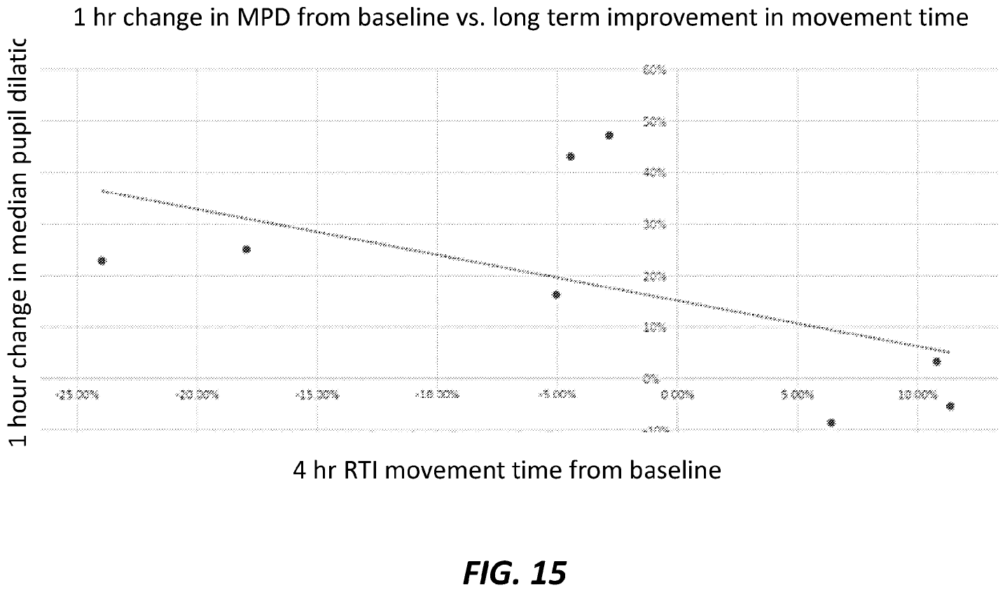

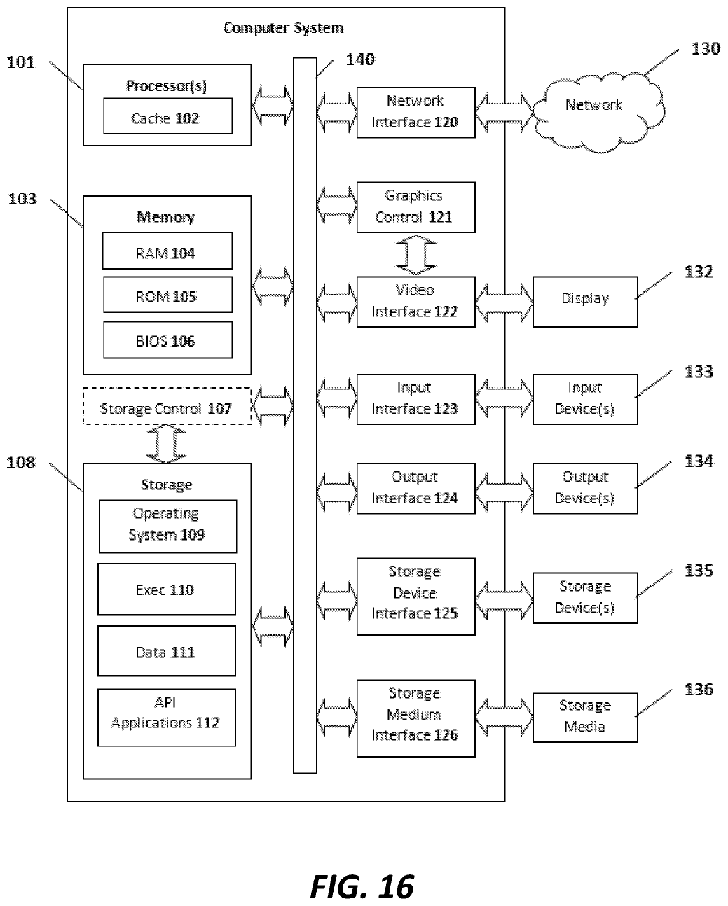

In some embodiments, RTI, STAI or pupillometry are used to identify low responders. In some embodiments, any combination of RTI, STAI, or pupillometry is used to identify low responders. In some embodiments, both RTI and pupillometry are used in combination to identify low responders, such as described in Example 2 (e.g. FIG. 15). In some embodiments, RTI and/or STAI are used to identify low responders, such as described in Example 2 (e.g. Table 1). In some embodiments, RTI, pupillometry, and STAI questionnaires are used in combination to identify low responders, such as described in example 2 (e.g. Table 1 and FIG. 15).

In some embodiments, the methods of the present application include measuring and identifying a state of an individual for the purposes of identifying a biomarker indicative of responsiveness to drug treatment (e.g. low responder). In some embodiments, the biomarker is used to identify high and low responding individuals. In some embodiments, the biomarker is useful in segregating individuals into treatable and non-treatable patient groups. In some embodiments, the methods herein are useful to identify those individuals that would benefit most from treatment. In some embodiments, the methods herein are useful as predictive tools for personalized and medical treatment. In some embodiments, the methods provided herein are useful in selecting patients for clinical trials. In some embodiments, the methods provided herein are useful for assessing eligibility of insurance coverage for individuals seeking drug treatment.

In certain embodiments, provided herein is a method of assessing and treating an individual comprising:
a. identifying or measuring a biomarker of the individual by performing an evaluation on the individual;
b. assessing the biomarker; and
c. if the biomarker is indicative of drug responsiveness, administering to the individual an active agent (e.g. 5-HT receptor agonist (e.g., psilocybin)), or a pharmaceutically acceptable salt, solvate, metabolite, derivative, or prodrug thereof.

Provided in some embodiments herein are methods for treating and individual suffering from a brain disease, disorder or condition (e.g., a mental, a behavioral, a neuropsychiatric, or the like, condition))). In some embodiments, provided herein are methods for treating or reducing the incidence of an episode in an individual susceptible to or suffering from a brain disease, disorder or condition (e.g., a mental, a behavioral, a neuropsychiatric, or the like, condition) by administering a dose (e.g., a therapeutically effective dose) of one or more 5-HT receptor agonist (e.g., psilocybin) to the individual.

In some embodiments, the evaluation is performed by the individual (e.g., in or outside a healthcare setting (e.g., in a natural environment, such as, for example, at home, at work, or the like)). In some embodiments, the evaluation is performed by a heath care provider (e.g., doctor, nurse, clinician).

Provided in some embodiments herein is a method for treating or reducing the incidence of a brain disorder or condition, and/or symptoms thereof, in an individual (e.g., in need thereof).

Provided in some embodiments herein is a method for treating or reducing the incidence of a mental, a behavioral, or a neuropsychiatric condition, and/or symptoms thereof, in an individual (e.g., in need thereof).

Provided in some embodiments herein is a method for treating attention deficit hyperactivity disorder (ADHD) or attention deficit disorder (ADD) in an individual (e.g., in need thereof).

Provided in some embodiments herein is a method for treating mild cognitive impairment, dementia, or Alzheimer's disease in an individual (e.g., in need thereof).

Provided in some embodiments herein is a method for improving coping in an individual (e.g., in need thereof). In some embodiments, coping is psychological coping. In some embodiments, the method is for improving stress recovery rate in the individual. In some embodiments, the individual is suffering from or susceptible to stress.

Provided in some embodiments herein is a method for increasing motivation in an individual (e.g., in need thereof). In some embodiments, the individual is suffering from or susceptible to low motivation, anxiety, apathy, fear, phobia, constructive impulsivity, depression, or the like. In some embodiments, the low motivation is induced by stress, anxiety, or the like.

Provided in some embodiments herein is a method for treating addiction in an individual (e.g., in need thereof).

Provided in some embodiments herein is a method for treating brain inflammation (e.g., encephalitis) or brain fog in an individual (e.g., in need thereof). In some embodiments, the brain inflammation or brain fog is secondary to a concussion, traumatic brain injury (TBI) (e.g., mild TBI (mTBI)), or the like. In some embodiments, the brain inflammation or brain fog is secondary to a natural insult (e.g., injury, stroke, or the like). In some embodiments, the individual is suffering from or susceptible to brain inflammation (e.g., encephalitis) or brain fog.

In some embodiments, the evaluation is identified or measured by performing a brain evaluation on the individual. In some embodiments, the brain evaluation is an electroencephalogram (EEG), a magnetoencephalogram (MEG), functional near-infrared spectroscopy (fNIRS), Positron Emission Tomography (PET), or a transcranial functional ultrasound imaging. In some embodiments, the evaluation is an EEG.

In some embodiments, the evaluation (e.g. brain test) is performed using a wearable device (e.g brain imaging). In some embodiments, the evaluation is recorded to a local memory or over a network using a mobile device (e.g., through a mobile application). In some embodiments, the method comprises patient reported outcomes or subjective mood, affect, coping, sleep quality, stress, anxiety, memory, and other emotional or functional data. In some embodiments, the method further comprises providing data (e.g., patterns, clusters, classifications, amplitudes, frequencies, or magnitude) on a device (e.g., a phone, tablet, or computer) to the individual and/or a caregiver (e.g., physicians, counselors, psychologist, or spiritual leader), the data comprising visual representations of brain responses or brain activity data and/or survey or patient reported outcomes to help convey the effectiveness or lack thereof of a psychedelic therapy.

In some embodiments, the method comprises performing one or more heart test (e.g., to determine heart rate or cardiovascular activity) on the individual (e.g., for at least 30 seconds) (e.g., the one or more heart test being measured using a separate electrode sensitive voltage or sensor (e.g., photodiode) sensing an optical measure from visible light or infrared light). In some embodiments, the method comprises performing one or more test to measure blood pressure or respiratory rate.

In some embodiments, the method comprises performing one or more test to measure skin conductance or impedance.

In some embodiments, the method comprises measurement of a biological sample taken from the individual. In some embodiments, the biological sample is serum, plasma, whole blood, urine, or the like.

In some embodiments, the method is for treating a mental, behavioral, or neuropsychiatric condition, or the symptoms thereof. In some embodiments, the method is for managing a mental, a behavioral, or a neuropsychiatric condition, or the symptoms thereof. In some embodiments, the method is for treating and managing a mental, behavioral, or neuropsychiatric condition, or the symptoms thereof. In some embodiments, the individual is suffering from or susceptible to the mental, a behavioral, or a neuropsychiatric condition. In certain instances, the symptoms of the mental, a behavioral, or a neuropsychiatric condition are physical, behavioral, emotional, mental, or a combination thereof.

In some embodiments, the mental, the behavioral, or the neuropsychiatric condition is any disease or disorder provided herein. In some embodiments, the mental, the behavioral, or the neuropsychiatric condition is a symptom (e.g., provided herein) of any disease or disorder provided herein. In some embodiments, the mental, the behavioral, or the neuropsychiatric condition is a chronic condition.

In some embodiments, the mental, the behavioral, or the neuropsychiatric condition is a Diagnostic and Statistical Manual of Mental Disorders (DSM-5) category disease or disorder). In some embodiments, the mental, the behavioral, or the neuropsychiatric condition is a non-DSM-5 category disease or disorder.

In some embodiments, the mental, the behavioral, or the neuropsychiatric condition is an attention (e.g., an attention deficit) or a cognitive (e.g., neurocognitive) disorder or condition. In some embodiments, the mental, the behavioral, or the neuropsychiatric condition is a neurocognitive disorder or condition. In some embodiments, the mental, the behavioral, or the neuropsychiatric condition is an attention deficit disorder or condition. In some embodiments, the cognitive condition is mild cognitive impairment, dementia, or Alzheimer's disease.

In some embodiments, the mental, the behavioral, or the neuropsychiatric condition is induced by stress and/or anxiety.

In some embodiments, the mental, the behavioral, or the neuropsychiatric condition is selected from the group consisting of addiction, anxiety, apathy, attention (e.g., the lack thereof), and depression (e.g., moderate depression). In some embodiments, the mental, the behavioral, or the neuropsychiatric condition is PTSD, constructive impulsivity, a phobia, a fear, or the like. In some embodiments, the mental, the behavioral, or the neuropsychiatric condition is major depressive disorder.

In some embodiments, the individual is susceptible to or suffering from a brain disorder or condition (e.g., a mental condition, a behavioral condition, a neuropsychiatric condition, a brain state or a lack thereof (e.g., coping, motivation, stress, depression or anxiety), encephalitis, or a brain dysfunction (e.g., cognitive decline or brain fog).

In some embodiments, the condition is attention deficit hyperactivity disorder (ADHD) or attention deficit disorder (ADD).

In some embodiments, the condition is mild cognitive impairment, dementia, or Alzheimer's disease.

In some embodiments, the condition is addiction.

In various embodiments, methods provided herein are suitable for treating any suitable disorder, such as a neurological condition, such as a neurological disorder, or symptoms thereof. In specific embodiments, the neurological condition is a neurocognitive disorder. In various embodiments, methods provided herein are suitable for treating any suitable disorder or symptoms thereof including, but not limited to, feelings of distress, futility, disempowerment, despair, helplessness, and the like. In some embodiments, the disorder causing the feelings of distress, futility, disempowerment, despair, helplessness, and the like is a demoralization disorder. In some embodiments, symptoms of the neurological condition are physical, behavioral, emotional, mental or a combination thereof. In some embodiments, the neurological condition is an addictive disorder (e.g., alcohol abuse, substance abuse, smoking, or obesity). In some embodiments, the neurological condition is an eating disorder or an auditory disorder. In some embodiments, the neurological condition is pain (e.g., chronic pain). In some embodiments, the neurological condition is depression, bipolar disorder, post-traumatic stress disorder (PTSD), panic disorder, phobia, schizophrenia, psychopathy, or antisocial personality disorder. In some embodiments, the neurological condition is an impulsive disorder. In some embodiments, the impulsive disorder is attention deficit hyperactivity disorder (ADHD), Tourette's syndrome or autism. In some embodiments, the neurological condition is a compulsive disorder (e.g., obsessive compulsive disorder (OCD), gambling, or aberrant sexual behavior). In some embodiments, the neurological condition is a personality disorder (e.g., conduct disorder, antisocial personality, or aggressive behavior).

In some embodiments, administration of an active agent to a subject in need thereof occurs several times per day. In some embodiments, administration to a subject in need thereof occurs no more frequently than once a day (e.g., no more frequently than once every other day, no more frequently than once every third day, no more frequently than twice a week, no more frequently than once a week, no more frequently than once every two weeks, or the like). In some embodiments, administration to a subject in need thereof occurs once a day, every alternate day, three times a week, twice a week, once a week, every other week, two weeks per month, three weeks per month, once a month, twice a month or three times per month. In specific embodiments, administration is about once a day. In other specific embodiments, administration is about every alternate day. In still other specific embodiments, administration is about once a week.

In various embodiments, administration continues for any suitable length of time, such as at least one day, at least one week, at least 1 month, 2 months, 3 months, 4 months, 5 months, 6 months, 7 months, 8 months, 9 months, 10 months, 11 months, 12 months, 18 months, 2 years, 3 years, or more.

Provided in some embodiments here is a method comprising measuring (e.g., on a network (e.g., WiFi, cloud, BLE, 3/4/5G)) one or more brain response using a node or a wearable brain imaging device (e.g., an EEG), wherein the patient listens to paired or sustained auditory stimuli (e.g., at home, at work, or at school), the auditory stimuli being delivered from an application on a (e.g., mobile) device (e.g., cellular phone, tablet, or the like).

Provided in some embodiments herein is a method of imaging brain activity (e.g., voltage) in an individual using a wearable device (e.g., EEG (e.g., multi-electrode EEG device)), the wearable device having two or more electrodes that make contact with the forehead of the individual for at least thirty seconds and up to twenty minutes.

Provided in some embodiments herein is a method for optically imaging brain activity in an individual (e.g., from a wearable device) to determine, the predicted effectiveness of a psychedelic treatment, wherein a brain signal is identified by transmission of visible light (400-680 nanometers) or infrared light (greater than or equal to 680 nanometers) across the skin into the brain to collect reflected photons by a sensor (e.g., a photodiode or CMOS).

Provided in some embodiments is a method of acoustically imaging brain activity in an individual (e.g., from a wearable device) to determine the predictive effectiveness of a psychedelic treatment, wherein a brain signal is identified by transmission of ultrasound (e.g., having an acoustic frequency of greater than or equal to one megahertz) (e.g., across the skin and skull) into the brain to collect reflected ultrasound waves by a sensor (e.g., a piezoelectric resonant material (e.g., a PZT, a CMUT, a PMUT).

In some embodiments, the method comprises using artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory) for determining the effectiveness of a treatment paradigm or a patient to undergo treatment with psychedelic substances (e.g. psilocybin).

Provided in some embodiments herein is a computer-implemented method for determining or predicting the effectiveness of a psychedelic treatment administered to an individual.

Provided in some embodiments herein is a computer-implemented method for determining or predicting the effectiveness of a 5-HT receptor agonist in an individual. In some embodiments, the method comprises emitting one or more (e.g., auditory or visual) to the individual. In some embodiments, the method comprises receiving, from a brain imaging device (e.g., an EEG), a brain response.

In some embodiments, the method comprises receiving, from the individual, an emotional data. In some embodiments, the emotional data comprises a mood rating, a sleep rating, a stress rating, an anxiety rating, a memory rating, or any combination thereof.

In some embodiments, two or more of any step provided herein are performed simultaneously.

In some embodiments, two or more of any step provided herein are performed sequentially.

In some embodiments, the outcome of the evaluation is transmitted by a mobile device (e.g. cellular phone, tablet). In some embodiments, the outcome is transmitted over a wireless network (e.g. WiFi, cloud, BLE, 3/4/5G). In some embodiments, the outcome is determined by a machine learning algorithm.

Provided in some embodiments herein is a computer-implemented system for determining the outcome.

In some embodiments, the system comprises a digital processing device. In some embodiments, the digital processing device comprises at least one processor. In some embodiments, the digital processing device comprises an operating system configured to perform executable instructions. In some embodiments, the digital processing device comprises a memory. In some embodiments, the digital processing device comprises a computer program including instructions executable by the digital processing device to create an application.

Provided in some embodiment herein is a non-transitory computer-readable storage media encoded with a computer program including instructions executable by a processor to create an application for the method.

Provided in some embodiment herein is a non-transitory computer-readable storage media encoded with a computer program including instructions executable by a processor to create an application for the method.

In some embodiments, the application is configured to direct a device (e.g., an auditory or visual device) to emit one or more (auditory or visual) stimulus to the individual. In some embodiments, the application is configured to receive, from a brain imaging device (e.g. EEG), a brain response. In some embodiments, the application is configured to receive, from the individual, an emotional data. In some embodiments, the emotional data comprises a mood rating, a sleep rating, a stress rating, an anxiety rating, a memory rating, or any combination thereof.

In some embodiments, the application is configured to simultaneously perform two or more of steps provided herein.

In some embodiments, the application is configured to sequentially perform two or more of steps provided herein.

In some embodiments, the emotional data comprises data obtain from one or more questionnaires regarding, for example, depression, anxiety, stress, coping, mood, sleep, and quality of life of the individual.

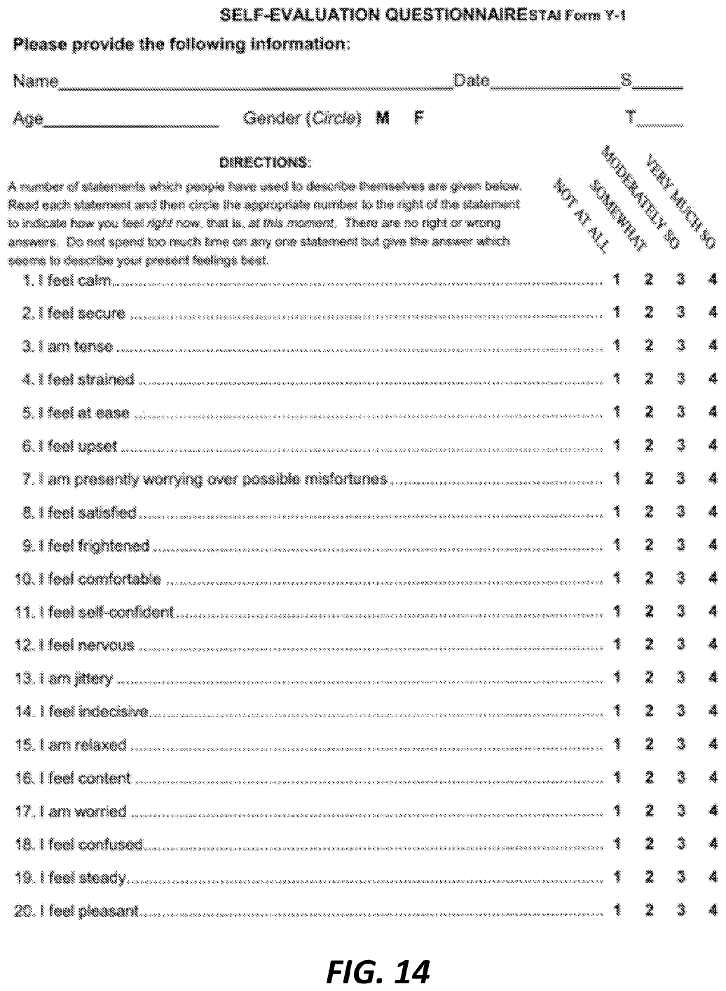

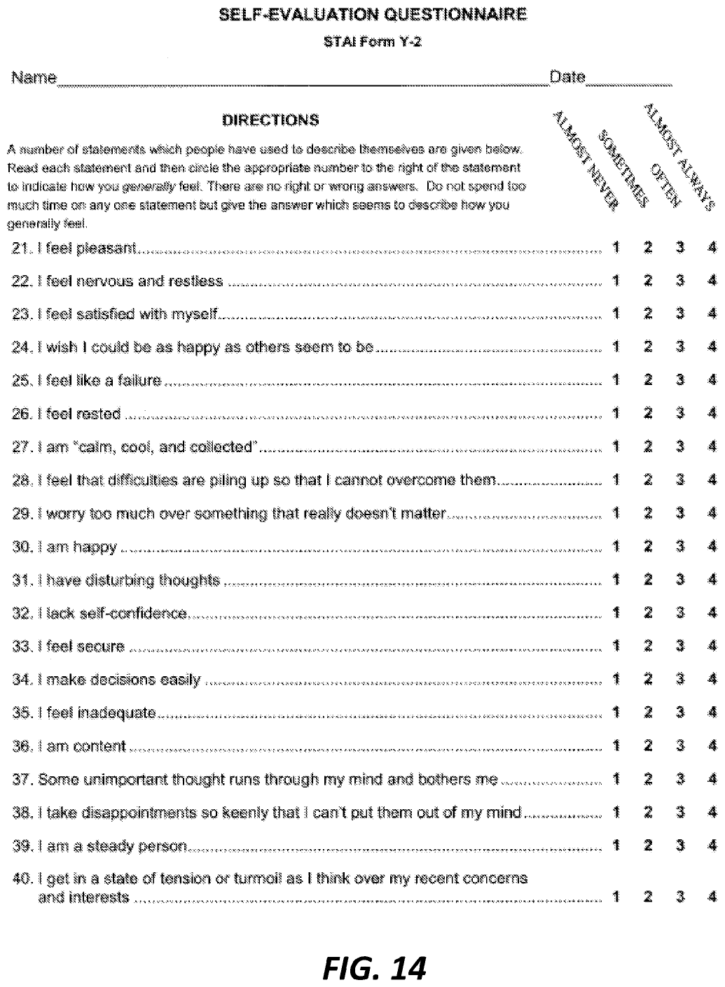

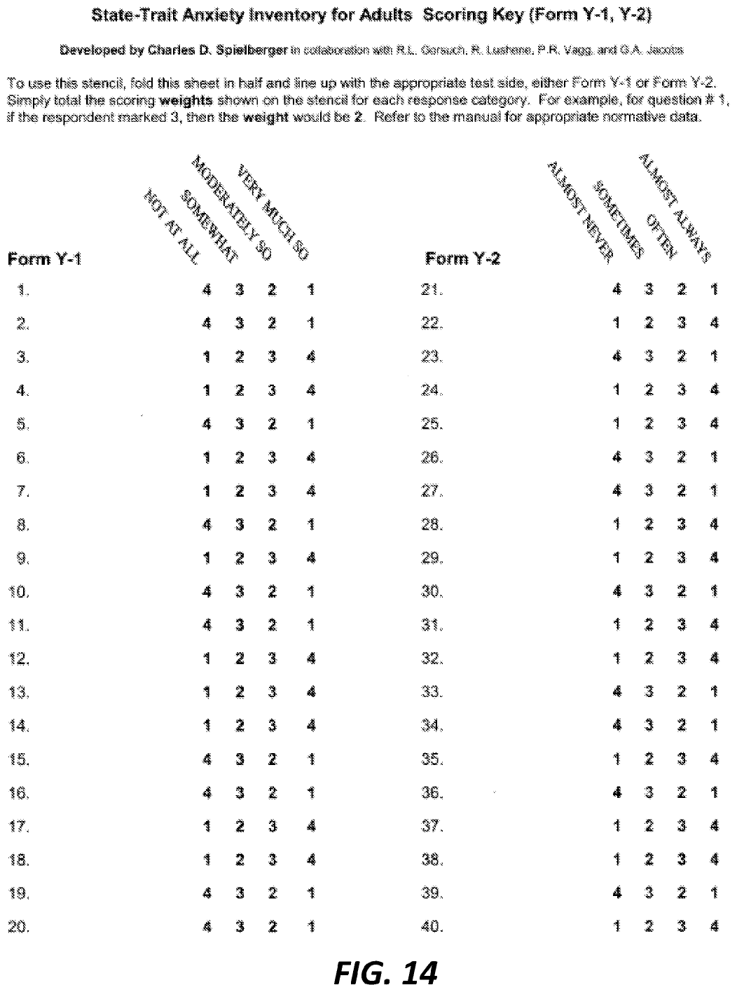

In some embodiments, the one or more questionnaires can include a Beck Depression Inventory (BDI), a Generalized Anxiety Disorder (GAD-7), a Depression Anxiety Stress Scale (DASS), a Brief-COPE, a Positive and Negative Affect Schedule (PANAS), a State Trait Anxiety Inventory (STAI) (e.g. STAI-S and/or STAI-T), a modified version of a Russel Mood Circumplex, a modified version of a NIH Sleep Diary, a 36 Item Short Form Health Survey (SF-36), a 5D Altered state of Consciousness Scale (5d-ASC), quality of life enjoyment and satisfaction questionnaire-short form (Q-LES-Q-SF), mini international neuropsychiatric interview (MINI), diagnostic assessment research tool (DART), Montgomery-Asberg Depression Rating Scale (MADRS), impact of event scale-revised (IES-R), and/or a daily sleep diary questionnaire. In some embodiments, the questionnaire is a mini-international neuropsychiatric interview (MINI). In some embodiments, the questionnaire is the diagnostic assessment research tool (DART). In some embodiments, the questionnaire, is the Montgomery-Asberg Depression Rating Scale (MADRS). In some embodiments, the questionnaire is the impact of event scale revised (IES-R). In some embodiments, the questionnaire is STAI (e.g., STAI-S and/or STAI-T), such as described in Example 2 (FIG. 14).

In some embodiments, identifying or measuring a state is performed by administering to the individual a questionnaire (e.g., STAI). In some embodiments, identifying or measuring a state is performed by administering to the individual an STAI (e.g., STAI-S and/or STAI-T) questionnaire. In some embodiments, identifying or measuring a state is performed by administering to the individual an STAI-S (e.g., STAI state anxiety subscale) questionnaire. In some embodiments, identifying or measuring a state is performed by administering to the individual an STAI-T (e.g., STAI trait anxiety subscale) questionnaire. In some instances, each question on the STAI questionnaire is rated on a 4-point scale (e.g., not at all, somewhat, moderately so, very much so). In some instances, the range of possible scores from the STAI Form Y spans from a minimum score of 20 to a maximum score of 80 on both the STAI-T and STAI-S subscales. In some embodiments, STAI (e.g., STAI-T and/or STAI-S) scores are measured before and after administration of an active agent. In some embodiments, STAI (e.g., STAI-T and/or STAI-S) scores are evaluated as a comparison between baseline (e.g., before administration) and score after treatment (e.g., 2 hours after treatment or 4 hours after treatment) to assess treatment efficacy or responsiveness. In some instances, an STAI score of 20-37 is classified as no to low anxiety. In some instances, an STAI score of 38-44 is classified as moderate anxiety. In some instances, an STAI score of 45-80 is classified as high anxiety.

In some instances, such as described in Example 2, baseline anxiety scores as measured by STAI (e.g., STAI-S and/or STAI-T) may be used to identify “low-responders” who may respond to treatment with an active agent provided herein.

In some embodiments, the application is configured to perform receiving (e.g., at least once weekly for 5 or more weeks) an individual's responses to the one or more questionnaires.

In some embodiments, the application is configured to perform scoring the individual's responses to the one or more questionnaires.

In some embodiments, the application is configured to perform receiving resting state brain activity from the individual. In some embodiments, the resting state brain activity is measured for at least thirty seconds (e.g., at least once, and up to four times, daily). In some embodiments, identifying the therapeutically effective dose of the 5-HT receptor agonist is based at least in part on changes to the resting state brain activity.

In some embodiments, receiving, from a brain imaging device (e.g., EEG), a brain response, comprises receiving amplitude and spectral power of EEG potentials measured from frontal, temporal, and parietal EEG sites in response to the one or more auditory and/or visual stimulus. In some embodiments, the one or more auditory stimulus comprises one or more auditory tasks comprising P50 paired click auditory suppression, Mismatch Negativity (MMN), and/or Auditory Steady State Response (ASSR). In some embodiments, the one or more visual stimulus comprises one or more visual tasks comprising an Emotional Flanker Task and/or a Continuous Performance Test (CPT).

In some embodiments, the evaluation uses one or more statistical methods comprising Bayesian methods, Mixed Model Repeated Measures (MMRM), repeated measures ANOVA and ANCOVA, and regression analyses.

In some embodiments, the individual is suffering from or susceptible to low motivation (e.g., low motivation induced by stress or anxiety), anxiety, apathy, fear, phobia, constructive impulsivity, or depression). In some embodiments, the individual is suffering from or susceptible to addiction.

In some embodiments, the method provided herein is a method for treating attention deficit hyperactivity disorder (ADHD) or attention deficit disorder (ADD) in an individual (e.g., in need thereof).

In some embodiments, the method provided herein is a method for treating mild cognitive impairment, dementia, or Alzheimer's disease in an individual (e.g., in need thereof).

In some embodiments, the method provided herein is a method for treating addiction (e.g., kleptomania, pyromania, addictive disorders, substance dependence, substance abuse, alcoholism, drug addiction, opioid addiction, cocaine addiction, gambling addiction, tobacco dependence, food addiction, or the like) in an individual (e.g., in need thereof).

Provided in some embodiments herein is a method for treating brain inflammation (e.g., encephalitis) or brain fog (e.g., associated with concussion, traumatic brain injury (TBI) (e.g., mild TBI (mTBI) (e.g., following (natural) insult (e.g., injury or stroke)), in an individual (e.g., in need thereof).

In some embodiments, the individual is suffering from or susceptible to brain inflammation (e.g., encephalitis) or brain fog.

In some embodiments, the evaluation is a brain imaging test. In some embodiments, the evaluation is selected from the group consisting of an electroencephalogram (EEG), a magnetoencephalogram (MEG), functional near-infrared spectroscopy (fNIRS), PET, and a transcranial functional ultrasound imaging. In some embodiments, the evaluation is selected from the group consisting of an electroencephalogram (EEG), a magnetoencephalogram (MEG), functional near-infrared spectroscopy (fNIRS), PET, and a transcranial functional ultrasound imaging. In some embodiments, the brain evaluation and the other brain evaluation are the same type of brain test. In some embodiments, the evaluation is an EEG. In some embodiments, the PET or fNRIS is used to identify target receptor occupancy in the brain. In some embodiments, the target receptor is one or more 5HT receptor. In some embodiments, the PET or fNRIS are used in accordance with the methods disclosed herein to PET to provide personalized care to patients that are either high or low responding individuals.

In some embodiments, the evaluation is an initial (e.g., baseline) brain imaging test performed prior to subjecting the individual to the one or more stimulus.

In some embodiments, the one or more stimulus comprises one or more auditory stimulus. In some embodiments, the one or more stimulus is one or more auditory stimulus.

In some embodiments, the one or more auditory stimulus comprises a first auditory stimulus and a second auditory stimulus. In some embodiments, the one or more auditory stimulus is a paired auditory stimuli.

In some embodiments, the first auditory stimulus and the second auditory stimulus are successive.

In some embodiments, the one or more auditory stimulus has a frequency of at least 20 Hertz (Hz) (e.g., 50 or more, 100 Hz or more, 200 Hz or more, 300 Hz or more, 400 Hz or more, 500 Hz or more, 600 Hz or more, 700 Hz or more, 800 Hz or more, 900 Hz or more, 1,000 Hz or more, or 5,000 Hz or more). In some embodiments, the one or more auditory stimulus has a frequency of at most 10,000 Hertz (Hz) (e.g., 10,000 Hz or less, 5,000 Hz or less, 1,000 Hz or less, 900 Hz or less, 800 Hz or less, 700 Hz or less, 600 Hz or less, 500 Hz or less, 400 Hz or less, 300 Hz or less, 200 Hz or less, or 100 Hz or less). In some embodiments, the one or more auditory stimulus has a frequency of 20 Hz to 10,000 Hz. In some embodiments, the one or more auditory stimulus has a frequency of 100 Hz to 1,000 Hz. In some embodiments, the one or more auditory stimulus has a frequency of 300 Hz to 700 Hz. In some embodiments, the one or more auditory stimulus has a frequency of 300 Hz. In some embodiments, the one or more auditory stimulus has a frequency of 700 Hz.

In some embodiments, the first auditory stimulus and the second auditory stimulus have the same acoustic frequency (e.g., 300 Hertz (Hz) and 300 Hz). In some embodiments, the one or more auditory stimulus comprises matched paired tones. In some embodiments, the first auditory stimulus and the second auditory stimulus have a different acoustic frequency (e.g., 300 Hz and 700 Hz). In some embodiments, the one or more auditory stimulus comprises mismatched paired tones.

In some embodiments, the second auditory stimulus is at least 10 milliseconds (ms) (e.g., 20 ms or more, 30 ms or more, 50 ms or more, 100 ms or more, 200 ms or more, 300 ms or more, 400 ms or more, 500 ms or more, 600 ms or more, 700 ms or more, 800 ms or more, 900 ms or more, 1,000 ms or more, 5,000 ms or more, 10,000 ms or more, or 100,000 ms or more) after the first auditory stimulus. In some embodiments, the second auditory stimulus is at most 10,000 ms (e.g., 10,000 ms or less, 5,000 ms or less, 1,000 ms or less, 900 ms or less, 800 ms or less, 700 ms or less, 600 ms or less, 500 ms or less, 400 ms or less, 300 ms or less, 200 ms or less, 100 ms or less, 50 ms or less, or 10 ms or less) after the first auditory stimulus. In some embodiments, the second auditory stimulus is 10,000 ms to 10 ms after the first auditory stimulus. In some embodiments, the second auditory stimulus is 1,000 ms to 100 ms after the first auditory stimulus.

In some embodiments, the time period between the first auditory stimulus and the second auditory stimulus is fixed. In some embodiments, the time period between the first auditory stimulus and the second auditory stimulus is random.

In some embodiments, the one or more auditory stimulus is one or more sustained auditory stimulus.

In some embodiments, the one or more sustained auditory stimulus occurs for at least 1 ms (e.g., 1 ms or more, 10 ms or more, 50 ms or more, 100 ms or more, 200 ms or more, 300 ms or more, 400 ms or more, 500 ms or more, 600 ms or more, 700 ms or more, 800 ms or more, 900 ms or more, 1,000 ms or more, 5,000 ms or more, 10,000 ms or more, or 50,000 ms or more). In some embodiments, the one or more sustained auditory stimulus occurs for at most 60,000 ms (e.g., 50,000 ms or less, 10,000 ms or less, 5,000 ms or less, 1,000 ms or less, 900 ms or less, 800 ms or less, 700 ms or less, 600 ms or less, 500 ms or less, 400 ms or less, 300 ms or less, 200 ms or less, 100 ms or less, 50 ms or less, 10 ms or less, or 1 ms or less). In some embodiments, the one or more sustained auditory stimulus occurs for 1 ms to 60,000 ms. In some embodiments, the one or more sustained auditory stimulus occurs for 100 ms to 1,000 ms. In some embodiments, the one or more sustained auditory stimulus occurs for 500 ms.

In some embodiments, the one or more sustained auditory stimulus has a fixed acoustic frequency (e.g., occurring for a period of time of greater than or equal to about five hundred ms). In some embodiments, the one or more sustained auditory stimulus has a variable acoustic frequency (e.g., occurring for a period of time of greater than or equal to about five hundred ms).

In some embodiments, the one or more stimulus comprises one or more visual stimulus. In some embodiments, the one or more stimulus is one or more visual stimulus.

In some embodiments, the one or more visual stimulus is an image, a series of images, a movie (e.g., a .GIF), or a contrast. In some embodiments, the one or more visual stimulus is a shape, a pattern, an emotional face, a color, or the like. In some embodiments, the one or more visual stimulus is a series of shapes, a series of patterns, a series of emotional faces, or the like. In some embodiments, the one or more visual stimulus is a flash. In some embodiments, the one or more visual stimulus is a video.

In some embodiments, the one or more visual stimulus comprises a first visual stimulus and a second visual stimulus. In some embodiments, the one or more visual stimulus is a paired visual stimuli.

In some embodiments, the first visual stimulus and the second visual stimulus are successive.

In some embodiments, the one or more visual stimulus occurs for at least 10 milliseconds (ms) (e.g., 20 ms or more, 30 ms or more, 50 ms or more, 100 ms or more, 200 ms or more, 300 ms or more, 400 ms or more, 500 ms or more, 600 ms or more, 700 ms or more, 800 ms or more, 900 ms or more, 1 second(s) or more, 5 s or more, 10 s or more, 30 s or more, 1 minute (min) or more, 5 mins or more, 10 mins or more, 20 mins or more, 30 mins or more, or 1 hour or more). In some embodiments, the one or more visual stimulus occurs for at most 1 hour (e.g., 30 min or less, 20 min or less, 10 min or less, 5 min or less, 1 min or less, 50 s or less, 40 s or less, 30 s or less, 20 s or less, 10 s or less, 5 s or less, 1 s or less, 900 ms or less, 800 ms or less, 700 ms or less, 600 ms or less, 500 ms or less, 400 ms or less, 300 ms or less, 200 ms or less, 100 ms or less, 50 ms or less, 20 ms or less, or 10 ms or less). In some embodiments, the one or more visual stimulus occurs for 1 hour to 10 ms. In some embodiments, the one or more visual stimulus occurs for 10 ms to 1 s. In some embodiments, the one or more visual stimulus occurs for 1 min to 10 mins.

In some embodiments, the second visual stimulus is at least 10 milliseconds (ms) (e.g., 20 ms or more, 30 ms or more, 50 ms or more, 100 ms or more, 200 ms or more, 300 ms or more, 400 ms or more, 500 ms or more, 600 ms or more, 700 ms or more, 800 ms or more, 900 ms or more, 1,000 ms or more, 5,000 ms or more, 10,000 ms or more, or 100,000 ms or more) after the first visual stimulus. In some embodiments, the second visual stimulus is at most 10,000 ms (e.g., 10,000 ms or less, 5,000 ms or less, 1,000 ms or less, 900 ms or less, 800 ms or less, 700 ms or less, 600 ms or less, 500 ms or less, 400 ms or less, 300 ms or less, 200 ms or less, 100 ms or less, 50 ms or less, or 10 ms or less) after the first visual stimulus. In some embodiments, the second visual stimulus is 10,000 ms to 10 ms after the first visual stimulus. In some embodiments, the second visual stimulus is 1,000 ms to 100 ms after the first visual stimulus.

In some embodiments, the time period between the first visual stimulus and the second visual stimulus is fixed. In some embodiments, the time period between the first auditory stimulus and the second auditory stimulus is random.

In some embodiments, the one or more visual stimulus is one or more sustained visual stimulus.

In some embodiments, the one or more sustained auditory stimulus occurs for at least 1 ms (e.g., 1 ms or more, 10 ms or more, 50 ms or more, 100 ms or more, 200 ms or more, 300 ms or more, 400 ms or more, 500 ms or more, 600 ms or more, 700 ms or more, 800 ms or more, 900 ms or more, 1,000 ms or more, 5,000 ms or more, 10,000 ms or more, or 50,000 ms or more). In some embodiments, the one or more sustained auditory stimulus occurs for at most 60,000 ms (e.g., 50,000 ms or less, 10,000 ms or less, 5,000 ms or less, 1,000 ms or less, 900 ms or less, 800 ms or less, 700 ms or less, 600 ms or less, 500 ms or less, 400 ms or less, 300 ms or less, 200 ms or less, 100 ms or less, 50 ms or less, 10 ms or less, or 1 ms or less). In some embodiments, the one or more sustained auditory stimulus occurs for 1 ms to 60,000 ms. In some embodiments, the one or more sustained auditory stimulus occurs for 100 ms to 1,000 ms. In some embodiments, the one or more sustained auditory stimulus occurs for 500 ms.

In some embodiments, the first visual stimulus and the second visual auditory stimulus comprise shapes, patterns, emotional faces, and/or colors, and have the same contrast.

In some embodiments, the first visual stimulus and the second visual auditory stimulus comprise shapes, patterns, emotional faces, and/or colors, and have a different contrast.

In some embodiments, the individual is subjected to the one or more brain imaging test for at least once per day (e.g., twice per day or more, thrice per day or more, and so on). In some embodiments, the individual is subjected to the one or more brain imaging test for at least 1 s per day (e.g., 1 s per day or more, 10 s per day or more, 30 s per day or more, 1 min per day or more, 5 min per day or more, 10 min per day or more, or 30 min per day or more). In some embodiments, the individual is subjected to the one or more brain imaging test for 30 s per day to 30 mins per day. In some embodiments, the individual is subjected to the one or more brain imaging test for 1 min per day to 10 mins per day.

In some embodiments, the individual is subjected to the one or more brain imaging test at least one day (e.g., two days, three days, four days, five days, six days, or seven days) per week (e.g., for at least one week (e.g., for one week or more, two weeks or more, four weeks or more, and so on)).

In some embodiments, there is a lack of brain activity (e.g., compared to the first brain activity measurement). In some embodiments, the lack of brain activity is a resting state brain activity. In some embodiments, the resting state brain activity is measured across two conditions. In some embodiments, the two conditions are eyes open and eyes closed.

In some embodiments, the resting state brain activity is measured for at least 1 second (e.g., 1 s or more, 10 s or more, 20 s or more, 30 s or more, 40 s or more, 50 s or more, 1 min or more, 5 min or more, or 10 min or more). In some embodiments, the resting state brain activity is measured for at most 10 minutes (e.g., 10 min or less, 5 min or less, 1 min or less, 50 s or less, 40 s or less, 30 s or less, 20 s or less, 10 s or less, or 1 s or less). In some embodiments, the resting state brain activity is measured for 1 s to 10 min. In some embodiments, the resting state brain activity is measured for 1 s to 1 min. In some embodiments, the resting state brain activity is measured for 30 s.

In some embodiments, the resting state brain activity is measured daily, twice daily, thrice daily, every other day, three times per week, at least once per week, or the like. In some embodiments, the resting state brain activity is measured for one day, for one week, for one month, for one year, or the like.

In some embodiments, the brain activity or the lack thereof is measured in a time domain (e.g., as time-locked changes) to a paired auditory stimuli as a voltage (e.g., evoked potentials), a magnetic field, or a hemodynamic signal.

In some embodiments, the brain activity or the lack thereof is measured in a frequency domain as a voltage, a magnetic field, or a hemodynamic signal.

In some embodiments, the hemodynamic signal is a flux, a flow, a velocity, or an oxygenation level.

In some embodiments, the voltage is power in alpha bands, beta bands, gamma bands, delta bands, or theta bands.

The Delta band can represent low frequency brain activity associated with resting states, deep concentration, or sleep. When activity is high (warmer colors) in this band, the brain is in a resting or drowsy state. When activity in this band is low (cooler colors) then the brain is more active and alert.

The Theta band can be associated with a relaxed state of mind that is most active when one is conscious, but not engaging in learning or a specific task. For example, theta brain wave activity is higher when attention is inwardly focused like during meditation. When the brain is actively engaged in learning, problem solving, or outwardly focused then theta activity is lower.

The Alpha band can be associated with an active level of thought during which one is not actively processing information. Alpha consists of the everyday thoughts that flow through the mind when not concentrating. It is considered a learning state when one are alert but not actively processing.

The Beta band can be associated with active thought and information processing when one is focused on a task or outcome. High levels of Beta can reflect a busy mind or actively engaged brain state, whereas lower levels of Beta can reflect a calmer mind.

The Gamma band can reflect a deeper focused brain state where attention is locked on a task, deep concentration, or problem solving. High gamma activity can also reflect synchronous processing of multiple streams of information in the brain. Lower levels of gamma activity can reflect that the brain is not locked into working on a specific task or solving a specific problem at that moment.

In some embodiments, the method comprises performing one or more heart test (e.g., to determine heart rate or cardiovascular activity) on the individual. In some embodiments, the heart test is performed for an amount of time sufficient to determine the cardiovascular activity (e.g., heart rate) of the individual (e.g., for at least 30 seconds).

In some embodiments, the method comprises performing one or more test to measure skin conductance or impedance.

In some embodiments, the method comprises integrating electronic patient reported outcomes or subjective mood, affect, coping, sleep quality, stress, anxiety, memory, and/or other emotional or functional data with brain imaging or brain activity levels.

In some embodiments, the further comprises providing data (e.g., patterns, clusters, classifications, amplitudes, frequencies, or magnitude) on a device (e.g., a phone, tablet, or computer) to the individual and/or a caregiver (e.g., physicians, counselors, psychologist, or spiritual leader). In some embodiments, the data comprises visual representations of brain activity data and/or survey or patient reported outcomes.

Provided herein are methods for managing or treating disorders or conditions, or treating symptoms of disorders or conditions, in an individual (e.g., in need thereof), comprising administering one or more 5-HT receptor agonists, or pharmaceutically acceptable salts, solvates, metabolites, derivatives, or prodrugs thereof.

In some embodiments, the individual is in need of a treatment for the brain disorder or condition. In some embodiments, the individual is in need of a treatment or a reduction in the incidence of the mental, the behavioral, or the neuropsychiatric condition, and/or the symptoms thereof. In some embodiments, the individual is susceptible to or suffering from a brain disorder or condition (e.g., a mental condition, a behavioral condition, a neuropsychiatric condition, a brain state or a lack thereof (e.g., coping, motivation, stress, depression or anxiety), encephalitis, or a brain dysfunction (e.g., cognitive decline or brain fog). In some embodiments, the individual is susceptible to or suffering from the mental, behavioral, or neuropsychiatric condition, and/or the symptoms thereof. In some embodiments, the individual is suffering from or susceptible to stress. In some embodiments, the individual is suffering from or susceptible to depression, anxiety, cognitive decline, or the like.

In some embodiments, the disorders or conditions are neurological disorders or conditions. In some embodiments, the disorders or conditions are neurocognitive disorders or conditions. In some embodiments, the disorders or conditions are neurodegenerative disorders or conditions. In some embodiments, the symptoms of the neurological condition are physical, behavioral, emotional, mental, or a combination thereof.

In some embodiments, the mental, the behavioral, or the neuropsychiatric condition is an attention condition or a cognitive (e.g., neurocognitive) condition. In some embodiments, the mental, the behavioral, or the neuropsychiatric condition (e.g., a Diagnostic and Statistical Manual of Mental Disorders (DSM-5) category or non-DSM-5 category disease or disorder) is selected from the group consisting of addiction, anxiety (e.g., post-traumatic stress disorder (PTSD), constructive impulsivity, a phobia, or fear), apathy, and depression (e.g., major depressive disorder).

In some preferred embodiments, the attention condition is attention deficit hyperactivity disorder (ADHD) or attention deficit disorder (ADD).

In some embodiments, the symptoms of the mental, a behavioral, or a neuropsychiatric condition are physical, behavioral, emotional, mental, or a combination thereof.

Provided in some instances herein is a method for increasing motivation in an individual. In some embodiments, the individual is suffering from or susceptible to low motivation (e.g., as a symptom of a neurocognitive or neurodevelopmental disorder), apathy, fear, phobia, constructive impulsivity, attention (e.g., or the lack thereof), cognitive conditions, or depression, comprising administering one or more 5-HT receptor agonists, or pharmaceutically acceptable salts, solvates, metabolites, derivatives, or prodrugs thereof.

In certain preferred embodiments, the cognitive condition is mild cognitive impairment, dementia, or Alzheimer's disease.

Provided herein are methods for managing or treating disorders, conditions or symptoms including but not limited to addiction (disorders), such as but not limited to alcohol abuse, substance abuse, smoking, or eating disorders. Provided herein are methods for managing or treating disorders, conditions or symptoms including but not limited to eating disorders and auditory disorders. Provided herein are methods for managing or treating disorders, conditions or symptoms including but not limited to pain, such as but not limited to chronic pain. Provided herein are methods for managing or treating disorders, conditions or symptoms including but not limited to depression, bipolar disorder, post-traumatic stress disorder (PTSD), panic disorder, phobia, schizophrenia, psychopathy, or antisocial personality disorder. Provided herein are methods for managing or treating disorders, conditions or symptoms including but not limited to impulse disorders, such as but not limited to attention deficit hyperactivity disorder (ADHD), Tourette's syndrome or autism. Provided herein are methods for managing or treating disorders, conditions or symptoms including but not limited to compulsive disorder, such as but not limited to obsessive compulsive disorder (OCD), gambling, or aberrant sexual behavior. Provided herein are methods for managing or treating disorders, conditions or symptoms including but not limited to personality disorders, such as but not limited to conduct disorder, antisocial personality, or aggressive behavior.

Further examples of the disorders, conditions and symptoms which may be managed or treated include, by way of non-limiting example:

Neurodevelopmental Disorders, such as but not limited to attention-deficit/hyperactivity disorder (ADHD), autism spectrum disorder, learning disorders and the like.

Schizophrenia Spectrum and other Psychotic Disorders, including but not limited to detachment from reality, delusions, hallucinations, and disorganized thinking and speech.

Bipolar and Related Disorders which may involve episodes of mania (periods of excessive excitement, activity, and energy) alternating with periods of depression.

Depressive Disorders which may involve feelings of extreme sadness, reduced interest in previously enjoyable activities, including but not limited to depression, severe depression, major depressive disorder (MDD), premenstrual dysphoric disorder (PMDD) and the like.

Anxiety Disorders which may involve worrying excessively about potential bad things or situations. Examples include generalized anxiety disorder (GAD), panic disorder and phobias (irrational fears of specific things) and the like. Provided herein are methods for managing or treating generalized anxiety disorder (GAD). In some embodiments, individuals with GAD are identified using the Quality of Life Enjoyment and Satisfaction Questionnaire-Short Form (Q-LES-Q-SF). In some embodiments, the effects (e.g., objective and/or subjective) of a 5-HT receptor agonist (e.g., psilocybin or psilocin) are assessed by measuring electroencephalogram (EEG) resting state, such as described in Example 3. In some embodiments, an individual has a GAD-7 score of ≥10 and a CGI-S score ≥4, such as before treatment with a 5-HT receptor agonist (e.g., psilocybin or psilocin).

Obsessive-Compulsive and Related Disorders which may involve repeated, unwanted urges, thoughts, or images (obsessions) and feeling driven to taking repeated actions in response to them (compulsions). Non-limiting examples include obsessive-compulsive disorder (OCD), hoarding disorder, extreme nail biting, and hair-pulling disorder (trichotillomania).

Trauma and Stressor-Related Disorders which may develop during or after stressful or traumatic life events. Non-limiting examples include posttraumatic stress disorder (PTSD) and acute stress disorder.

Dissociative Disorders wherein the sense of self is may be disrupted, such as but not limited to dissociative identity disorder, dissociative amnesia and the like.

Somatic Symptom and Related Disorders which may involve distressing and incapacitating physical symptoms with no clear medical cause. Non-limiting examples include illness anxiety disorder, somatic symptom disorder (hypochondriasis), factitious disorder and the like.

Feeding and Eating Disorders which may involve disturbances related to eating, such as but not limited to anorexia nervosa, bulimia nervosa, and binge eating disorder.

Elimination Disorders which may involve inappropriate elimination (release) of urine or stool by accident or deliberately, such as but not limited to bedwetting (enuresis).

Sleep-Wake Disorders which may involve severe sleep disorders, including but not limited to insomnia disorder, nightmare disorder, sleep apnea, and restless legs syndrome.

Disruptive, Impulse-Control, and Conduct Disorders which may involve difficulty with emotional and/or behavioral self-control, such as but not limited to kleptomania (repeated stealing), pyromania, and intermittent explosive disorder.

Substance Related addiction Disorders which may involve problems associated with excessive use of substances such as alcohol (alcohol dependence, alcoholism), tobacco products, drugs, opioids (for example, cocaine, oxycodone, morphine and the like), recreational drugs, hallucinogens and the like.

Addictive Disorders which may involve problems associated with excessive use of particular behaviors or fixations, such as but not limited to gambling disorder.

Neurocognitive Disorders which may affect the ability to think and reason, such as but not limited to traumatic brain injury (TBI), Alzheimer's disease and the like.

Personality Disorders which may involve enduring patterns of emotional instability and unhealthy behaviors that disrupt daily living and relationships. Examples include but are not limited to borderline, antisocial, and narcissistic personality disorders.

Gender Dysphoria which may involve distress caused by a person's desire to be a different gender.

Sexual Dysfunctions such as but not limited to premature ejaculation, erectile disorder, and female orgasmic disorder.

Paraphilic Disorders (sexual perversion, sexual deviation) which may involve sexual interest in atypical objects, situations, fantasies, behaviors, or individuals. Examples include but are not limited to sexual sadism disorder, voyeuristic disorder, and pedophilic disorder.

Further examples of the disorders, conditions and symptoms which may be managed or treated include by way of non-limiting examples Fragile X syndrome, Downs's syndrome, migraine headache, cluster headache, psychiatric disorders, neurodevelopmental disorders, attention-deficit/hyperactivity disorder (ADHD), autism spectrum disorder, learning disorders, schizophrenia spectrum, psychotic disorders, bipolar disorders, depression, severe depression, major depressive disorder (MDD), premenstrual dysphoric disorder (PMDD), suicidality, mood related disorders, panic disorder, panic attack, phobias, agoraphobia, selective mutism, obsessive-compulsive disorder (OCD), hoarding disorder, hair-pulling disorder (trichotillomania), excoriation (skin-picking) disorder, substance-/medication-induced obsessive-compulsive disorder, trauma related disorders, traumatic brain injury (TBI), posttraumatic stress disorder (PTSD), acute stress disorder, dissociative disorders, dissociative identity disorder, dissociative amnesia, anxiety, anxiety disorders, generalized anxiety disorder (GAD), social anxiety disorder, separation anxiety disorder, illness anxiety disorders, somatic disorders and diseases, somatic symptom disorder (hypochondriasis), factitious disorder, feeding disorders, eating disorders, anorexia, anorexia nervosa, bulimia nervosa, binge eating disorder, elimination disorders, enuresis, sleep disorders, insomnia, nightmare disorder, sleep apnea, central sleep apnea, narcolepsy, obstructive sleep apnea, hypopnea, and sleep-related hypoventilation, restless legs syndrome, jet lag, sexual dysfunction, premature ejaculation, erectile disorder, female orgasmic disorder, gender identity disorder, gender dysphoria, disruptive disorders, impulse-control disorders, conduct disorders, disruptive conduct disorders, impulse-control disorders, oppositional defiant disorder (ODD), aggression, kleptomania, pyromania, addictive disorders, substance dependence, substance abuse, alcoholism, drug addiction, opioid addiction, cocaine addiction, gambling addiction, tobacco dependence, food addiction, other forms of addiction to substances and behaviors, obesity, cognitive disorders, memory related disorders, learning related disorders, neurocognitive disorders, Alzheimer's disease, personality disorders, narcissistic personality disorders, Asperger syndrome, Tourette syndrome, Huntington's disease, Parkinson's disease, Lewy body disease, amyotrophic lateral sclerosis (ALS), Friedreich's ataxia, muscular atrophy, prion disease, dementia, vascular dementia, dementia/neurocognitive issues due to infection, dementia due to substance abuse or exposure to toxins, frontotemporal degeneration, mood disorders, delirium, aphasia, apraxia, agnosia, concussion, amnesia, anterograde amnesia, retrograde amnesia, body dysmorphic disorder, reactive attachment disorder, Fragile X syndrome, Down syndrome, migraines, migraine headache, cluster headache, cardiovascular disease, inflammatory conditions, fibromyalgia and pain.

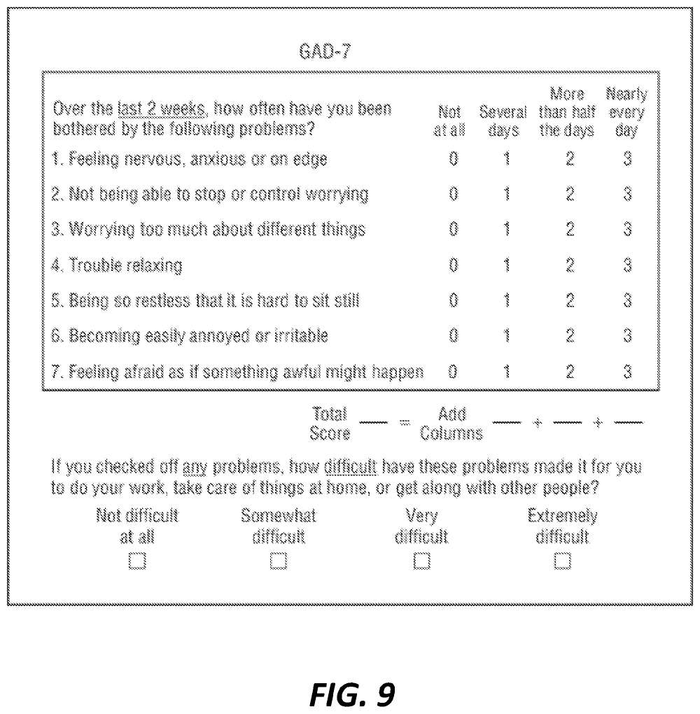

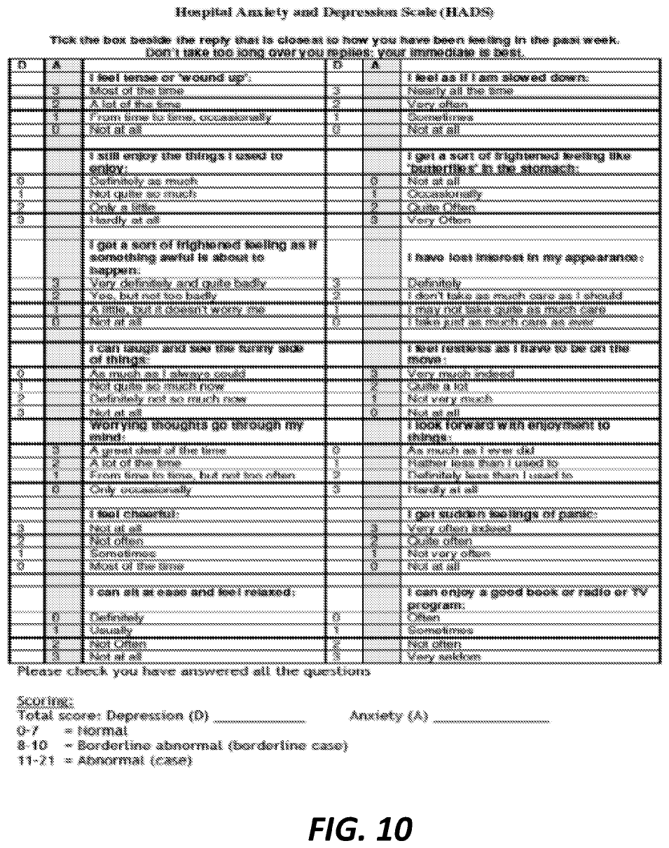

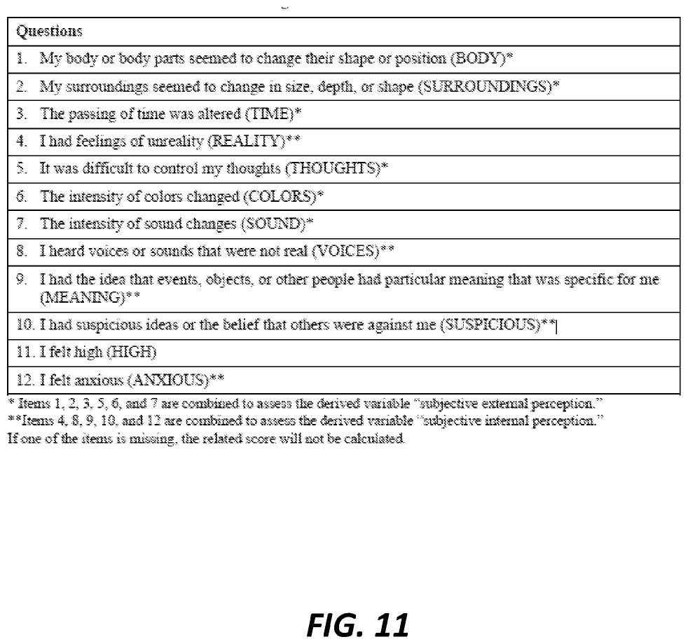

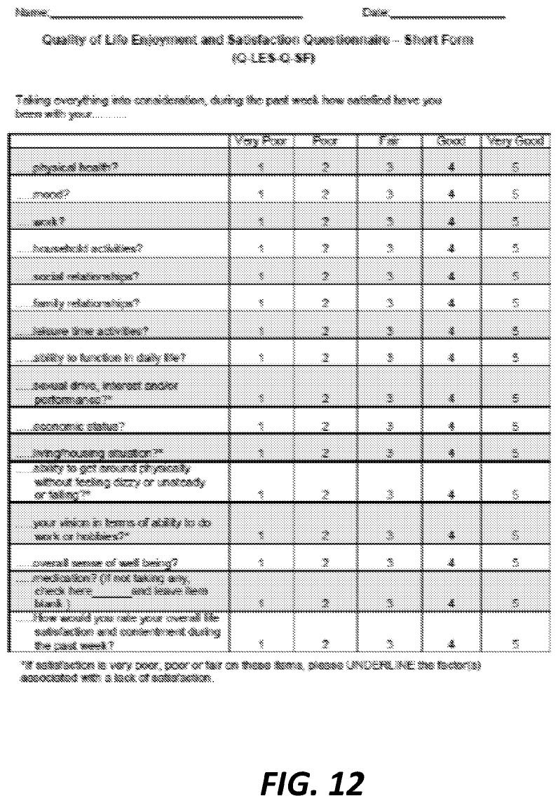

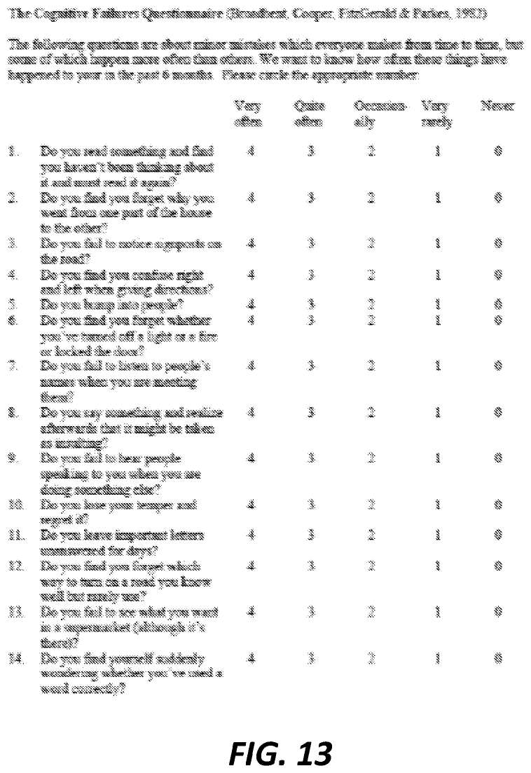

In some embodiments, outcomes of treatment with a 5-HT receptor agonist (e.g., psilocybin) provided herein are assessed using the generalized anxiety disorder scale (GAD-7) (FIG. 9). In some embodiments, outcomes of treatment with a 5-HT receptor agonist (e.g., psilocybin) provided herein are assessed using the Hospital Anxiety and Depression Scale (HADS) (FIG. 10). In some embodiments, outcomes of treatment with a 5-HT receptor agonist (e.g., psilocybin) provided herein are assessed using the Clinical Global Impressions Scale (CGI). In some embodiments, outcomes of treatment with a 5-HT receptor agonist (e.g., psilocybin) provided herein are assessed using the State-Trait Anxiety Inventory (STAI) (e.g. STAI-S and/or STAI-T). In some embodiments, outcomes of treatment with a 5-HT receptor agonist (e.g., psilocybin) provided herein are assessed using the Bowdle Visual Analog Scale (FIG. 11). In some embodiments, outcomes of treatment with a 5-HT receptor agonist (e.g., psilocybin) provided herein are assessed using the Quality of Life Enjoyment and Satisfaction Questionnaire—Short Form (Q-LES-Q-SF) (FIG. 12). In some embodiments, outcomes of treatment with a 5-HT receptor agonist (e.g., psilocybin) provided herein are assessed using Cognitive Battery—Digital-Symbol Substitution Task, Stroop Colour and Word Test, and Cognitive Failures Questionnaire (FIG. 13). In some embodiments, outcomes of treatment with a 5-HT receptor agonist (e.g., psilocybin) provided herein are assessed using electroencephalogram (EEG). In some embodiments, outcomes of treatment with a 5-HT receptor agonist (e.g., psilocybin) provided herein are assessed using drug concentration measurements.

In some embodiments, any pharmaceutical composition or formulation or 5-HT receptor agonist agent disclosed herein is administered for therapeutic application. In some embodiments, the pharmaceutical composition or formation or 5-HT receptor agonist agent is administered once per day, twice per day, three times per day or more. In certain instances, the pharmaceutical composition or formulation or 5-HT receptor agonist agent is administered daily, every day, every alternate day, five days a week, once a week, every other week, two weeks per month, three weeks per month, once a month, twice a month, three times per month, or more. In some embodiments, the pharmaceutical composition or formulation or 5-HT receptor agonist agent is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are co-administered. In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap of about 0.5 hour, 1 hour, 2 hour, 3 hour, 12 hours, 1 day (e.g., 24 hours), 2 days, 3 days, or more between administrations.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some embodiments, described herein are compositions and formulations comprising a 5-HT receptor agonist active ingredient. In some embodiments, the formulation comprising 5-HT receptor agonist, or pharmaceutically acceptable salt, solvate, metabolite, derivative, or prodrug thereof, has: an enhanced bioavailability and efficacy, a lower administration dose, a lower cytotoxicity, decreased side effects, or the like.

In some embodiments, the formulation comprises one or more 5-HT receptor agonist, or a pharmaceutically acceptable salt, solvate, metabolite, derivative, or prodrug thereof. In some embodiments, the one or more 5-HT receptor agonist, or a pharmaceutically acceptable salt, solvate, metabolite, derivative, or prodrug thereof is a 5HT_{2A} receptor agonist.

In some embodiments, the one or more 5-HT receptor agonist, or the pharmaceutically acceptable salt, solvate, metabolite, derivative, or prodrug thereof, is a hallucinogenic compound (e.g., wherein the hallucinogenic compound produces a hallucinogenic effect (e.g., an adverse event, a clinically important effect (e.g., clinically important impairment of the individual, altered (e.g., visual, auditory, body, time and space) perception, altered cognition, impaired attention, drowsiness, and/or confusion)) in the individual in need thereof at or above the hallucinogenic threshold (e.g., at or above a Cₘₐₓ above the hallucinogenic effective threshold)).

In some embodiments, the one or more 5-HT receptor agonist is psilocybin or psilocin, or a pharmaceutically acceptable salt, solvate, metabolite, derivative, or prodrug thereof. In some embodiments, the one or more 5-HT receptor agonist is psilocin.

In some embodiments, provided herein is a formulation configured to maintain a level of an active 5-HT receptor agonist at or above a minimum therapeutically effective threshold of the active 5-HT receptor agonist in an individual (e.g., in a biological sample (e.g., serum, plasma, or whole blood) of the individual) for more than or equal to two hours (e.g., more than 2 hours, more than 12 hours, more than 1 day, more than 7 days, or more than 14 days). In some embodiments, provided herein is a formulation configured to maintain a level of an active 5-HT receptor agonist below a hallucinogenic threshold (e.g., below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like) of the active 5-HT receptor agonist in an individual (e.g., in a biological sample (e.g., serum, plasma, or whole blood) of the individual) for more than or equal to two hours (e.g., more than 2 hours, more than 12 hours, more than 1 day, more than 7 days, or more than 14 days). In some embodiments, provided herein is a formulation configured to maintain a level of an active 5-HT receptor agonist (i) at or above a minimum therapeutically effective threshold and (ii) below a hallucinogenic threshold (e.g., below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like) of the active 5-HT receptor agonist in the individual (e.g., in a biological sample (e.g., serum, plasma, or whole blood) of the individual) for more than or equal to two hours (e.g., more than 2 hours, more than 12 hours, more than 1 day, more than 7 days, or more than 14 days).

In some embodiments, provided herein is a formulation configured to release the active 5-HT receptor agonist at a dose sufficient to provide a Cₘₐₓ below the hallucinogenic effective threshold of the active 5-HT receptor agonist and a Cₘᵢₙ of at least the therapeutically effective threshold of the active 5-HT receptor agonist in the individual.

In some embodiments, provided herein is a formulation configured to provide a maximum plasma concentration (Cₘₐₓ) of the active 5-HT receptor agonist of about 0.1 ng/ml to about 20 ng/mL (e.g., about 0.5 ng/mL to about 20 ng/ml, about 1 ng/ml to about 15 ng/mL, about 2 ng/ml to about 12 ng/mL, or the like) in the individual.

In some embodiments, provided herein is a formulation configured to provide a plasma concentration of the active 5-HT receptor agonist of at least 0.1 ng/ml (e.g., at least 0.2 ng/ml, at least 0.3 ng/mL, at least 0.5 ng/ml, or the like) in the individual for at least 3 hours (e.g., at least 12 hours, at least 24 hours, at least 36 hours, at least 48 hours, at least 72 hours, at least 96 hours, at least 120 hours, at least 144 hours, or the like).

In some embodiments, the composition or formulation is an oral formulation. In some instances, the oral formulation comprising 5-HT receptor agonist, or pharmaceutically acceptable salt, solvate, metabolite, derivative, or prodrug thereof, has: an enhanced bioavailability and efficacy, a lower administration dose, a lower cytotoxicity, and decreased side effects.

In some embodiments, the pharmaceutically acceptable excipient is selected from the group consisting of fillers, binders, suspending agents, disintegrants, lubricants, and combinations thereof.

In some embodiments, the active agent (e.g., one or more 5-HT receptor agonist (e.g., psilocybin)) is administered orally followed by about 200 ml of water.

In some embodiments, 5HT receptor agonists or pharmaceutical compositions or formulations described herein are administered to a subject by multiple administration routes, including but not limited to, oral, parenteral (e.g. intravenous, subcutaneous, intramuscular), intranasal, inhalation, buccal, topical, rectal, or transdermal administration routes.

In some embodiments, the one or more 5-HT receptor agonist, or a pharmaceutically acceptable salt, solvate, metabolite, derivative, or prodrug thereof, is administered to the individual in need thereof as an oral formulation, an intravenous formulation, a dermal formulation, a buccal formulation, a nasal formulation, or an inhalation formulation.

In some embodiments, pharmaceutical compositions described herein, which include 5HT receptor agonist, or pharmaceutically acceptable salt, solvate, metabolite, derivative, or prodrug thereof, are formulated into any suitable dosage form.

In some embodiments, the pharmaceutical composition for oral use is a tablet, a pill, a powder, a capsule, solid dispersion, solid solution, bioerodible dosage form, pellets, granules, or an aerosol, or the like.

For oral administration, the pharmaceutical compositions disclosed herein are, in some instances, formulated readily by combining the active compound(s) with pharmaceutically acceptable carriers. In some instances, such formulations comprise pharmaceutically acceptable carriers including solid diluents or fillers, sterile aqueous media and various non-toxic organic solvents. In some instances, the compositions disclosed herein are present at concentration levels of at least about 0.5% by weight of the total composition of oral dosage forms, in an amount sufficient to provide a desired unit of dosage.

In some embodiments, the amount of 5HT receptor agonist, or pharmaceutically acceptable salt, solvate, metabolite, derivative, or prodrug thereof in a pharmaceutical composition provided herein is at least 1 mg/mL (e.g., 1 mg/mL or more, 5 mg/mL or more, 10 mg/mL or more, 15 mg/mL or more, 20 mg/mL or more, 25 mg/mL or more, or 30 mg/mL or more). In some embodiments, the amount of 5HT receptor agonist, or pharmaceutically acceptable salt, solvate, metabolite, derivative, or prodrug thereof in a pharmaceutical composition provided herein is at most 30 mg/ml (e.g., 30 mg/mL or less, 25 mg/mL or less, 20 mg/mL or less, 15 mg/mL or less, 10 mg/mL or less, 5 mg/mL or less, or 1 mg/mL or less). In some embodiments, the amount of 5HT receptor agonist, or pharmaceutically acceptable salt, solvate, metabolite, derivative, or prodrug thereof in a pharmaceutical composition provided herein is 1 mg/mL to 30 mg/mL. In some embodiments, the amount of 5HT receptor agonist, or pharmaceutically acceptable salt, solvate, metabolite, derivative, or prodrug thereof in a pharmaceutical composition provided herein is about 0.1 mg/ml to about 10 mg/ml.

In some embodiments, the amount of 5HT receptor agonist, or pharmaceutically acceptable salt, solvate, metabolite, derivative, or prodrug thereof in a pharmaceutical composition provided herein is at least about 0.001 mg (e.g., 0.001 mg or more, 0.01 mg or more, 0.1 mg or more, 1 mg or more, 10 mg or more, or 100 mg or more) In some embodiments, the amount of 5HT receptor agonist, or pharmaceutically acceptable salt, solvate, metabolite, derivative, or prodrug thereof in a pharmaceutical composition provided herein is at most about 100 mg (e.g., 100 mg or less, 10 mg or less, 1 mg or less, 0.1 mg or less, 0.01 mg or less, or 0.001 mg or less). In some embodiments, the amount of 5HT receptor agonist, or pharmaceutically acceptable salt, solvate, metabolite, derivative, or prodrug thereof in a pharmaceutical composition provided herein is about 0.001 mg to about 100 mg. In some embodiments, the amount of 5HT receptor agonist, or pharmaceutically acceptable salt, solvate, metabolite, derivative, or prodrug thereof in a pharmaceutical composition provided herein is about 0.005 mg to about 15 mg. In some embodiments, the amount of 5HT receptor agonist, or pharmaceutically acceptable salt, solvate, metabolite, derivative, or prodrug thereof in a pharmaceutical composition provided herein is about 0.01 mg to about 5 mg. In some embodiments, the amount of 5HT receptor agonist, or pharmaceutically acceptable salt, solvate, metabolite, derivative, or prodrug thereof in a pharmaceutical composition provided herein is about 0.05 mg to about 2.5 mg.

In some embodiments, the amount of 5HT receptor agonist, or pharmaceutically acceptable salt, solvate, metabolite, derivative, or prodrug thereof in a pharmaceutical composition provided herein is about 3 mg. In some embodiments, the amount of 5HT receptor agonist, or pharmaceutically acceptable salt, solvate, metabolite, derivative, or prodrug thereof in a pharmaceutical composition provided herein is about 0.5 mg to about 5 mg. In some embodiments, the amount of 5HT receptor agonist, or pharmaceutically acceptable salt, solvate, metabolite, derivative, or prodrug thereof in a pharmaceutical composition provided herein is about 0.5 mg to about 1 mg, about 0.5 mg to about 1.5 mg, about 0.5 mg to about 2 mg, about 0.5 mg to about 2.5 mg, about 0.5 mg to about 3 mg, about 0.5 mg to about 3.5 mg, about 0.5 mg to about 4 mg, about 0.5 mg to about 4.5 mg, about 0.5 mg to about 5 mg, about 1 mg to about 1.5 mg, about 1 mg to about 2 mg, about 1 mg to about 2.5 mg, about 1 mg to about 3 mg, about 1 mg to about 3.5 mg, about 1 mg to about 4 mg, about 1 mg to about 4.5 mg, about 1 mg to about 5 mg, about 1.5 mg to about 2 mg, about 1.5 mg to about 2.5 mg, about 1.5 mg to about 3 mg, about 1.5 mg to about 3.5 mg, about 1.5 mg to about 4 mg, about 1.5 mg to about 4.5 mg, about 1.5 mg to about 5 mg, about 2 mg to about 2.5 mg, about 2 mg to about 3 mg, about 2 mg to about 3.5 mg, about 2 mg to about 4 mg, about 2 mg to about 4.5 mg, about 2 mg to about 5 mg, about 2.5 mg to about 3 mg, about 2.5 mg to about 3.5 mg, about 2.5 mg to about 4 mg, about 2.5 mg to about 4.5 mg, about 2.5 mg to about 5 mg, about 3 mg to about 3.5 mg, about 3 mg to about 4 mg, about 3 mg to about 4.5 mg, about 3 mg to about 5 mg, about 3.5 mg to about 4 mg, about 3.5 mg to about 4.5 mg, about 3.5 mg to about 5 mg, about 4 mg to about 4.5 mg, about 4 mg to about 5 mg, or about 4.5 mg to about 5 mg. In some embodiments, the amount of 5HT receptor agonist, or pharmaceutically acceptable salt, solvate, metabolite, derivative, or prodrug thereof in a pharmaceutical composition provided herein is about 0.5 mg, 1 mg, 1.5 mg, 2 mg, 2.5 mg, 3 mg, 3.5 mg, 4 mg, 4.5 mg, or 5 mg. In some embodiments, the amount of 5HT receptor agonist, or pharmaceutically acceptable salt, solvate, metabolite, derivative, or prodrug thereof in a pharmaceutical composition provided herein is at least, or equal to about 0.5 mg, 1 mg, 1.5 mg, 2 mg, 2.5 mg, 3 mg, 3.5 mg, 4 mg, or 4.5 mg. In some embodiments, the amount of 5HT receptor agonist, or pharmaceutically acceptable salt, solvate, metabolite, derivative, or prodrug thereof in a pharmaceutical composition provided herein is at most, or equal to about 1 mg, 1.5 mg, 2 mg, 2.5 mg, 3 mg, 3.5 mg, 4 mg, 4.5 mg, or 5 mg. In some embodiments, the amount of 5HT receptor agonist, or pharmaceutically acceptable salt, solvate, metabolite, derivative, or prodrug thereof in a pharmaceutical composition provided herein is at most about 5 mg.

In some embodiments, the 5HT receptor agonist, or pharmaceutically acceptable salt, solvate, metabolite, derivative, or prodrug thereof in a pharmaceutical composition provided herein is formulated for oral administration. In some embodiments, the 5HT receptor agonist, or pharmaceutically acceptable salt, solvate, metabolite, derivative, or prodrug thereof in a pharmaceutical composition provided herein is formulated as an oral solution. In some embodiments, the 5HT receptor agonist, or pharmaceutically acceptable salt, solvate, metabolite, derivative, or prodrug thereof in a pharmaceutical composition provided herein is formulated as a solution, such as an oral solution, at a concentration of about 0.5 mg/mL. In some embodiments, the 5HT receptor agonist, or pharmaceutically acceptable salt, solvate, metabolite, derivative, or prodrug thereof in a pharmaceutical composition provided herein is formulated as a solution, such as an oral solution, at a concentration of about 0.05 mg/mL to about 1 mg/mL. In some embodiments, the 5HT receptor agonist, or pharmaceutically acceptable salt, solvate, metabolite, derivative, or prodrug thereof in a pharmaceutical composition provided herein is formulated as a solution at a concentration of about 0.05 mg/mL to about 0.1 mg/mL, about 0.05 mg/mL to about 0.25 mg/mL, about 0.05 mg/mL to about 0.5 mg/mL, about 0.05 mg/mL to about 0.75 mg/mL, about 0.05 mg/mL to about 1 mg/mL, about 0.1 mg/mL to about 0.25 mg/mL, about 0.1 mg/mL to about 0.5 mg/mL, about 0.1 mg/mL to about 0.75 mg/mL, about 0.1 mg/mL to about 1 mg/mL, about 0.25 mg/mL to about 0.5 mg/mL, about 0.25 mg/mL to about 0.75 mg/mL, about 0.25 mg/mL to about 1 mg/mL, about 0.5 mg/mL to about 0.75 mg/mL, about 0.5 mg/mL to about 1 mg/mL, or about 0.75 mg/mL to about 1 mg/mL. In some embodiments, the 5HT receptor agonist, or pharmaceutically acceptable salt, solvate, metabolite, derivative, or prodrug thereof in a pharmaceutical composition provided herein is formulated as a solution at a concentration of about 0.05 mg/mL, about 0.1 mg/mL, about 0.25 mg/mL, about 0.5 mg/mL, about 0.75 mg/mL, or about 1 mg/mL. In some embodiments, the 5HT receptor agonist, or pharmaceutically acceptable salt, solvate, metabolite, derivative, or prodrug thereof in a pharmaceutical composition provided herein is formulated as a solution at a concentration of at least, or equal to about 0.05 mg/mL, 0.1 mg/mL, 0.25 mg/mL, 0.5 mg/mL, or 0.75 mg/mL. In some embodiments, the 5HT receptor agonist, or pharmaceutically acceptable salt, solvate, metabolite, derivative, or prodrug thereof in a pharmaceutical composition provided herein is formulated as a solution at a concentration of at most, or equal to about 0.1 mg/mL, 0.25 mg/mL, 0.5 mg/mL, 0.75 mg/mL, or 1 mg/mL.

In some embodiments, the amount of 5HT receptor agonist, or pharmaceutically acceptable salt, solvate, metabolite, derivative, or prodrug thereof in a pharmaceutical composition provided herein is at least about 0.001 mg/kg (e.g., 0.001 mg/kg or more, 0.01 mg/kg or more, 0.1 mg/kg or more, 1 mg/kg or more, 10 mg/kg or more, or 100 mg/kg or more) In some embodiments, the amount of 5HT receptor agonist, or pharmaceutically acceptable salt, solvate, metabolite, derivative, or prodrug thereof in a pharmaceutical composition provided herein is at most about 100 mg/kg (e.g., 100 mg/kg or less, 10 mg/kg or less, 1 mg/kg or less, 0.1 mg/kg or less, 0.01 mg/kg or less, or 0.001 mg/kg or less). In some embodiments, the amount of 5HT receptor agonist, or pharmaceutically acceptable salt, solvate, metabolite, derivative, or prodrug thereof in a pharmaceutical composition provided herein is about 0.001 mg/kg to about 100 mg/kg. In some embodiments, the amount of 5HT receptor agonist, or pharmaceutically acceptable salt, solvate, metabolite, derivative, or prodrug thereof in a pharmaceutical composition provided herein is about 0.005 mg/kg to about 10 mg/kg. In some embodiments, the amount of 5HT receptor agonist, or pharmaceutically acceptable salt, solvate, metabolite, derivative, or prodrug thereof in a pharmaceutical composition provided herein is about 0.01 mg/kg to about 5 mg/kg. In some embodiments, the amount of 5HT receptor agonist, or pharmaceutically acceptable salt, solvate, metabolite, derivative, or prodrug thereof in a pharmaceutical composition provided herein is about 0.05 mg/kg to about 1 mg/kg.

In some embodiments, the 5HT receptor agonist, or pharmaceutically acceptable salt, solvate, metabolite, derivative, or prodrug thereof in a pharmaceutical composition provided herein is administered to an individual. In some embodiments, the 5HT receptor agonist, or pharmaceutically acceptable salt, solvate, metabolite, derivative, or prodrug thereof in a pharmaceutical composition provided herein is administered to an individual daily. In some embodiments, the 5HT receptor agonist, or pharmaceutically acceptable salt, solvate, metabolite, derivative, or prodrug thereof in a pharmaceutical composition provided herein is administered to an individual at a frequency of every three days (q3d). In some embodiments, the 5HT receptor agonist, or pharmaceutically acceptable salt, solvate, metabolite, derivative, or prodrug thereof in a pharmaceutical composition provided herein is administered to an individual at a frequency of once a day, such as once a day in the morning, once a day in the evening, or once every 24 hours. In some embodiments, the 5HT receptor agonist, or pharmaceutically acceptable salt, solvate, metabolite, derivative, or prodrug thereof in a pharmaceutical composition provided herein is administered to an individual at a frequency of more than once a day, such as twice a day, three times a day, or four times a day. In other embodiments, the 5HT receptor agonist, or pharmaceutically acceptable salt, solvate, metabolite, derivative, or prodrug thereof in a pharmaceutical composition provided herein is administered to an individual at a frequency of every 2 days, every 4 days, every 5 days, every 6 days, every 7 days, every 10 days, every 14 days, every 21 days, or every 28 days.

In some embodiments, the 5HT receptor agonist, or pharmaceutically acceptable salt, solvate, metabolite, derivative, or prodrug thereof in a pharmaceutical composition provided herein is administered to an individual at a frequency provided herein for 28 days. In some embodiments, the 5HT receptor agonist, or pharmaceutically acceptable salt, solvate, metabolite, derivative, or prodrug thereof in a pharmaceutical composition provided herein is administered to an individual at a frequency provided herein for 28 days or less (e.g. 1 day, 7 days, 14 days, 27 days, or any period in between). In some embodiments, the 5HT receptor agonist, or pharmaceutically acceptable salt, solvate, metabolite, derivative, or prodrug thereof in a pharmaceutical composition provided herein is administered to an individual at a frequency described herein for 56 days (e.g., 28 days with a 28 day extension). In some embodiments, the 5HT receptor agonist, or pharmaceutically acceptable salt, solvate, metabolite, derivative, or prodrug thereof in a pharmaceutical composition provided herein is administered to an individual at a frequency provided herein for 1 day to 112 days. In some embodiments, the 5HT receptor agonist, or pharmaceutically acceptable salt, solvate, metabolite, derivative, or prodrug thereof in a pharmaceutical composition provided herein is administered to an individual at a frequency provided herein for 1 day to 5 days, 1 day to 7 days, 1 day to 14 days, 1 day to 21 days, 1 day to 28 days, 1 day to 56 days, 1 day to 72 days, 1 day to 112 days, 5 days to 7 days, 5 days to 14 days, 5 days to 21 days, 5 days to 28 days, 5 days to 56 days, 5 days to 72 days, 5 days to 112 days, 7 days to 14 days, 7 days to 21 days, 7 days to 28 days, 7 days to 56 days, 7 days to 72 days, 7 days to 112 days, 14 days to 21 days, 14 days to 28 days, 14 days to 56 days, 14 days to 72 days, 14 days to 112 days, 21 days to 28 days, 21 days to 56 days, 21 days to 72 days, 21 days to 112 days, 28 days to 56 days, 28 days to 72 days, 28 days to 112 days, 56 days to 72 days, 56 days to 112 days, or 72 days to 112 days. In some embodiments, the 5HT receptor agonist, or pharmaceutically acceptable salt, solvate, metabolite, derivative, or prodrug thereof in a pharmaceutical composition provided herein is administered to an individual at a frequency provided herein for 1, 5, 7, 14, 21, 28, 56, 72, or 112 days. In some embodiments, the 5HT receptor agonist, or pharmaceutically acceptable salt, solvate, metabolite, derivative, or prodrug thereof in a pharmaceutical composition provided herein is administered to an individual at a frequency provided herein for at least 1, 5, 7, 14, 21 days, 28, 56, or 72 days. In some embodiments, the 5HT receptor agonist, or pharmaceutically acceptable salt, solvate, metabolite, derivative, or prodrug thereof in a pharmaceutical composition provided herein is administered to an individual at a frequency provided herein for at most 5, 7, 14, 21, 28, 56, 72, or 112 days. In some embodiments, the 5HT receptor agonist, or pharmaceutically acceptable salt, solvate, metabolite, derivative, or prodrug thereof in a pharmaceutical composition provided herein is administered to an individual at a frequency provided herein for up to 12 months. In some embodiments, the 5HT receptor agonist, or pharmaceutically acceptable salt, solvate, metabolite, derivative, or prodrug thereof in a pharmaceutical composition provided herein is administered to an individual at a frequency provided herein for years (e.g, 2, 3, 4 or 5 years). In some embodiments, the 5HT receptor agonist, or pharmaceutically acceptable salt, solvate, metabolite, derivative, or prodrug thereof in a pharmaceutical composition provided herein is administered to an individual at a frequency provided herein for 2 years to 5 years. In some embodiments, the 5HT receptor agonist, or pharmaceutically acceptable salt, solvate, metabolite, derivative, or prodrug thereof in a pharmaceutical composition provided herein is administered to an individual at a frequency provided herein for 2 to 3 years, 2 to 4 years, 3 to 4 years, or 4 to 5 years. In some embodiments, the 5HT receptor agonist, or pharmaceutically acceptable salt, solvate, metabolite, derivative, or prodrug thereof in a pharmaceutical composition provided herein is administered to an individual at a frequency provided herein for at least 3, 4, or 5 years. In some embodiments, the 5HT receptor agonist, or pharmaceutically acceptable salt, solvate, metabolite, derivative, or prodrug thereof in a pharmaceutical composition provided herein is administered to an individual at a frequency provided herein for at most 2, 3, or 4 years. In some embodiments, the 5HT receptor agonist, or pharmaceutically acceptable salt, solvate, metabolite, derivative, or prodrug thereof in a pharmaceutical composition provided herein is administered to an individual at a frequency provided herein as needed. In some embodiments, the 5HT receptor agonist, or pharmaceutically acceptable salt, solvate, metabolite, derivative, or prodrug thereof in a pharmaceutical composition provided herein is administered to an individual at a frequency provided herein for an indefinite period of time.

In some embodiments, the amount of 5-HT receptor agonist, or pharmaceutically acceptable salt, solvate, metabolite, derivative, or prodrugs thereof in a pharmaceutical composition provided herein is at least about 1% w/w (e.g., 1% or more, 10% or more, 20% or more, 30% or more, 40% or more, or 50% or more) (e.g., of the solids in an oral formulation). In some embodiments, the amount of 5-HT receptor agonist, or pharmaceutically acceptable salt, solvate, metabolite, derivative, or prodrugs thereof in a pharmaceutical composition provided herein is at most about 50% w/w (e.g., 50% w/w or less, 40% w/w or less, 30% w/w or less, 20% w/w or less, 10% w/w or less, or 1% w/w or less) (e.g., of the solids in an oral formulation).

In some embodiments, an evaluation (e.g., a brain test) provided herein is performed using a wearable brain imaging device (e.g., on the individual).

In some embodiments, one or more brain activity or the lack thereof is measured with the wearable brain imaging device and recorded to a local memory or over a network using a mobile device (e.g., through a mobile application).

In some embodiments, one or more cardiovascular activity (e.g., heart rate) is measured with an electrode sensitive voltage or sensor (e.g., a photodiode) sensing an optical measure from visible light or infrared light. In some embodiments, the electrode sensitive voltage or sensor is a separate electrode sensitive voltage or sensor (e.g., from the brain activity measuring device).

Provided in some embodiments herein is a method comprising measuring (e.g., on a network (e.g., WiFi, cloud, BLE, 3/4/5G)) one or more brain response using a node or a wearable brain imaging device (e.g., an EEG). In some embodiments, the patient listens to paired or sustained auditory stimuli (e.g., at home, at work, or at school). In some embodiments, the auditory stimuli being delivered from an application on a (e.g., mobile) device (e.g., cellular phone, tablet, or the like).

Provided in some embodiments herein is a method of imaging brain activity (e.g., voltage) in an individual using a wearable device (e.g., EEG (e.g., multi-electrode EEG device)), the wearable device having two or more electrodes that make contact with the forehead of the individual for at least thirty seconds and up to twenty minutes.

Provided in some embodiments herein is a method for optically imaging brain activity in an individual (e.g., from a wearable device) to determine or predict the effectiveness of a psychedelic compound, wherein a brain signal is identified by transmission of visible light (400-680 nanometers) or infrared light (greater than or equal to 680 nanometers) across the skin into the brain to collect reflected photons by a sensor (e.g., a photodiode or CMOS).

Provided in some embodiments herein is a method of acoustically imaging brain activity in an individual (e.g., from a wearable device) to determine or predict the effectiveness of a psychedelic compound, wherein a brain signal is identified by transmission of ultrasound (e.g., having an acoustic frequency of greater than or equal to one megahertz) (e.g., across the skin and skull) into the brain to collect reflected ultrasound waves by a sensor (e.g., a piezoelectric resonant material (e.g., a PZT, a CMUT, a PMUT).

In some embodiments, the method further comprises using artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory).

In some embodiments the method further comprises receiving, from the individual, an emotional data. In some embodiments, the emotional data comprises a mood rating, a sleep rating, a stress rating, an anxiety rating, a memory rating, or any combination thereof.

In some embodiments, the data is transmitted by a mobile device (e.g. cellular phone, tablet). In some embodiments, the therapeutically effective dose of the 5-HT receptor agonist is transmitted over a wireless network (e.g. WiFi, cloud, BLE, 3/4/5G). in some embodiments, the therapeutically effective dose of the 5-HT receptor agonist is determined by a machine learning algorithm.

Provided in some embodiments herein is a computer-implemented system for identifying a therapeutically effective dose of a 5-HT receptor agonist administered to an individual, the system comprising: a digital processing device comprising: at least one processor, an operating system configured to perform executable instructions, a memory, and a computer program including instructions executable by the digital processing device to create an application.

In some embodiments, the application is further configured to perform receiving, from the individual, an emotional data. In some embodiments, the emotional data comprises a mood rating, a sleep rating, a stress rating, an anxiety rating, a memory rating, or any combination thereof.

In some embodiments, the application is further configured to transmit the data to the individual, a caregiver, or both. In some embodiments, the application directs the transmission by a mobile device (e.g. cellular phone, tablet). In some embodiments, the application directs the transmission over a wireless network (e.g. WiFi, cloud, BLE, 3/4/5G). In some embodiments, the data is analyzed by a machine learning algorithm.

In some embodiments, the application can be used to monitor brain activity of subjects. The application can pair to a subject's device (e.g., EEG device). A subject can log onto the application using their subject ID. The application can be designed to acquire data regarding stress and anxiety using a standard STAI or modified version of the Short State Trait Anxiety Inventory (STAI-6) questionnaire once per day. The application can collect data on mood using a modified version of the Russel Mood Circumplex twice daily. The application can record information about sleep time and quality, fatigue, and drug use in the morning and evening using a modified version of the NIH Sleep Diary.

The application can work with a custom engineered, at home EEG device (e.g., a CGX, LLC device) that records activity from the prefrontal cortex using a wearable, low-risk device. The application can pair automatically to the device using Bluetooth. The rechargeable, battery-powered, wearable EEG device can use disposable, biocompatible, snap biopotential sensor electrodes. Users can record resting state at any time they wish in one-minute epochs. The option to record resting state brain activity in the morning and evening can be built into the questionnaires. Subjects may be asked to record 1-minute epochs of resting state EEG activity in the morning or evening at least 4 times per week. Subjects can record resting state EEG activity as often as desired.

The application can record information about sleep time and quality, fatigue, and drug use in the morning and evening using a modified version of the NIH Sleep Diary.

The application can also record information about coping. For example, the application can utilize an Approach-Avoid metric to measure coping.

In some embodiments, auditory stimuli comprises auditory tasks. Auditory tasks may comprise P50 paired click auditory suppression, Mismatch Negativity (MMN), and/or Auditory Steady State Response (ASSR). The P50 task can involve the subject listening to 120 paired auditory clicks (1 msec each) occurring 500 msec apart. Each pairing can occur 10 sec apart. The estimated time to complete the passive P50 task is 20 min. The ASSR task can require subjects to listen to a series of tones using a 500 or 1000 Hz carrier frequency modulated at 20 or 40 Hz for 1 second each. Each 1 sec tone can be separated by a three second inter-stimulus interval. A total of 100 tones at 20 and 40 Hz each can be delivered. The ASSR task can be expected to last 15 minutes. The MMN task can require subjects to listen to 500 tones (1 msec) spaced 1 second apart. There can be 400 tones at a standard frequency (750 Hz) and 100 randomly spaced odd-ball tones (1500 Hz). The MMN task can be expected to take 10 minutes. Subjects can also undergo a final resting state period of EEG collection where they lay down with their eyes closed for 10 minutes. EEG recordings will also include other biosensors for recording heart rate activity during testing.

In some embodiments, visual stimuli may include one or more visual tasks comprising an Emotional Flanker Task and/or a Continuous Performance Test (CPT). Following EEG assessments following auditory tasks as described above, subjects can undergo a series of computerized cognitive attention and emotional tasks while recording EEG. Subjects can first complete an Emotional Flanker Test that uses neutral faces or an angry face as a background distractor on classic flanker stimuli assessing congruent, incongruent, and neutral conditions. A stimulus from each condition can be presented 50 times and a total of 250 stimuli can be presented over 10 minutes. Subjects can then take a Conner's Continuous Performance Test that lasts 14 minutes to assess attention and cognitive control.

In some embodiments, the individual provided herein is restricted from taking medicines as described in Table 3. In some embodiments, the individual provide herein is restricted from taking SSRIs (e.g., sertraline, fluoxetine, citalopram escitalopram, vortioxetine), such as in an amount recommended for the treatment for anxiety disorders. In some embodiments, the individual provided herein is restricted from taking SNRIs (e.g., duloxetine, venlafaxine, desvenlafaxine), such as in an amount recommended for the treatment of anxiety disorder. In some embodiments, the individual provided herein is restricted from taking mirtrazapine such as in an amount of greater than 7.5 mg/day. In some embodiments, the individual provided herein is restricted from taking trazadone, such as in an amount of greater than 100 mg/day. In some embodiments, the individual provided herein is restricted from taking tricyclic antidepressants, such as in an amount recommended for anxiety. In some embodiments, the individual provided herein is restricted from taking quetiapine, such as in an amount of greater than 50 mg/day. In some embodiments, the individual provided herein is restricted from taking aripiprazole, such as in an amount of greater than 2 mg/day. In some embodiments, the individual provided herein is restricted from taking olanzapine, such as in an amount of greater than 2.5 mg/day. In some embodiments, the individual provided herein is restricted from taking brexpiprazole, such as in an amount of greater than 0.5 mg/day. In some embodiments, the individual provided herein is restricted from taking atypical antipsychotics with anxiolytic properties, such as daily. In some embodiments, the individual provided herein is restricted from taking pregabalin (e.g., when administered at anxiolytic doses), such as when administered in amounts of at least 100 mg/day.

In some instances, the individual provided herein meets DSM-V criteria for a primary diagnosis of generalized anxiety disorder, such as for at least 1 year, confirmed using the MINI and DART. In some instances, the individual provided herein has a GAD-7 score of at least 10, such as at screening. In some instances, the individual provided herein has a CGI-S score of at least 4, such as at screening. In some instances, the individual provided herein is non-pregnant and non-lactating. In some instances, the individual provided herein is surgically sterile for a minimum of 6 months, such achieved through hysterectomy, oophorectomy, or bilateral salpingectomy. In some instances, the individual provided herein is post-menopausal for a minimum of 1 year, such as confirmed by follicle-stimulating hormone test. In some instances, the individual provided herein agrees to avoid pregnancy and uses a medically acceptable method of contraception with male sexual partners, such as from at least 30 days prior to the study until 30 days after the study has ended. In some instances, the medically acceptable methods of contraception include double-barrier methods (e.g., male condom, spermicide with diaphragm or spermicide with cervical cap), oral contraceptives, hormonal patch, implant or injection, or hormonal or non-hormonal intrauterine device, such as in combination with a male condom with spermicide. In some instances, the individual provided herein is completely abstinent, such as when it is in line with the individual's preferred and usual lifestyle. In some instances, the individual provided herein who is able to father children uses medically acceptable methods of contraception, such as during treatment and 30 days after the last administration. In some instances, the individual provided herein refrains from sperm donation, such from first administration to at least 30 days after the last administration.

In some instances, the individual provided herein does not have a history of schizophrenia, bipolar affective disorder, delusional disorder, paranoid disorder, moderate or severe panic disorder, moderate or severe social anxiety disorder, schizoaffective disorder, moderate or severe obsessive-compulsive disorder, anorexia nervosa, bulimia nervosa, moderate or severe post-traumatic stress disorder (as assessed by the IES-R), moderate or severe personality disorder (Cluster B and Cluster C only), or moderate or severe MDD (as assessed by the MINI, DART, and scores on the MADRS). In some instances, the individual provided herein does have a history or presence of any clinically significant cardiac, endocrine, hematologic, hepatic, immunologic, metabolic, urologic, pulmonary, neurologic, dermatologic, renal, or other disease, such as at screening. In some instances, an individual provided herein does not take pharmacological treatment for GAD or depressive symptoms on a daily basis as outlined in Table 3. In some instances, the individual provided herein does not exhibit clinically significant abnormalities on ECG, including a QT interval corrected for heart rate (Bazett; QtCB interval) of greater than 440 milliseconds in males and greater tan 460 milliseconds in females. In some instances, an individual provided herein does not have a history of allergies to a 5HT receptor against described herein. In some instances, the individual provided herein does not have a history of seizures, family history of seizures, history of head trauma, history of neurosurgery, or close family history of idiopathic generalized epilepsy or other congenital epilepsies. In some instances, the individual provided herein does not have hepatitis B virus surface antigen, hepatitis C virus antibody, or human immunodeficiency virus (HIV) antibody. In some instances, the individual provided herein does not test positive at a urine drug screen, such as at screening. In some instances, the individual provided herein does not have a current or history of moderate or severe drug or alcohol use disorder (excluding alcohol and nicotine) within the past 2 years, or lifetime history of participation in a drug rehabilitation program (other than treatment for smoking cessation). In some instances, the individual provided herein does not or has not used CNS drugs with perception-altering properties (e.g., ketamine, lysergic acid diethylamide [LSD], phencyclidine [PCP], 3,4 methylenedioxymethamphetamine [MDMA], mescaline, psilocybin, tryptamine derivatives, or ring-substituted amphetamines with perception-altering effects) on more than 1 occasion for therapeutic or non-therapeutic purposes (i.e., for psychoactive effects) within the past 6 months. In some instances, the individual provided herein does not have a history of suicidal ideation in the past 12 months or active/current suicidality, based on C-SSRS results. In some instances, the individual provided herein does not currently use an investigational drug or device or has used such in 30 days prior to receiving the study drug, such as a 5HT receptor agonist provided herein. In some instances, the individual provided herein is not pregnant, breastfeeding, or has had a recent positive pregnancy test.

In some instances, the individual provided herein is required to abstain from alcohol for 48 hours prior to treatment, such as confirmed via a breath alcohol test. In some instances, the individual provided herein will be asked to abstain from strenuous physical activity for 48 hours prior to treatment and each inpatient study visit. In some instances, the individual provided herein will be asked to refrain from driving, operating machinery, or engaging in hazardous activities for approximately 4 hours after administration of the 5HT receptor agonist. In some instances, individuals provided herein are required to abstain from blood donation, such as during treatment and for 30 days after.

In some embodiments, an individual undergoes laboratory assessments before, after, or during treatment with a 5HT receptor agonist (e.g., psilocybin) as described herein. In some embodiments, an individual undergoes hematology assessments before, after, or during treatment with a 5HT receptor agonist (e.g., psilocybin) as described herein, such as hematocrit, hemoglobin, red blood cell count, total ad differential, (absolute) white blood cell count, or platelets. In some embodiments, an individual undergoes biochemistry assessments before, after, or during treatment with a 5HT receptor agonist (e.g., psilocybin) as described herein, such as sodium, potassium, glucose (random), creatinine, total protein, blood urea nitrogen, albumin, total bilirubin, alanine transferase, aspartate trans feast, lactic dehydrogenase, gamma-glutamyl transferase, alkaline phosphatase, creatine phosphokinase, or follicle stimulating hormone. In some embodiments, an individual undergoes urinalysis assessments before, after, or during treatment with a 5HT receptor agonist (e.g., psilocybin) as described herein, such as color, pH, specific gravity, ketones, protein, glucose, bilirubin, nitrite, urobilinogen, occult blood, microscopic examination of sediment (e.g., only if urinalysis dipstick results are abnormal).

In some embodiments, ventricular heart rate, PR, QRS, QT, QTcB, and/or QTcF intervals are assessed in the individual provided herein using ECG.

In some embodiments, the Columbia Suicide Severity Rating Scale is used to track suicidal events before treatment and during treatment of the individuals provided herein. In some embodiments, individuals who indicate high risk responses will discontinue treatment with the 5HT receptor agonists (e.g., psilocybin) provided herein.

In some instances, individuals provided herein complete a daily electronic diary, such as to record treatment compliance.

In some instances, adverse events include drug abuse, dependence, withdrawal, and substance-related disorders associated with the treatment with 5HT receptor agonists (e.g., psilocybin) described herein. In some instances, adverse events include perceptual disturbances or psychomimetic effects (e.g., hallucination, dissociation, confusional state). In some embodiments, adverse effects include mental and cognitive impairment (e.g., disturbance in attention, psychomotor skills impaired). In some embodiments, adverse effects include anxiety symptoms (e.g., insomnia, panic attack). In some embodiments, adverse effects include headache, suicidal thoughts, and thoughts of self-harm.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

### EXAMPLES

These examples are provided for illustrative purposes only and not to limit the scope of the claims provided herein.

### Example 1: Study 1: Effect of Psilocybin on Progressive Ratio Responding for Food and Identifying a “Low Responder”

To establish evidence for efficacy of low dose psilocybin and plasma psilocin exposure in a rat progressive ratio task and identifying a “low responder”.

Animals and housing: Male Long Evans rats were used (source: Charles River Laboratories, St. Constant, Quebec). All study animals were singly housed in a vivarium under a 12 h light:dark cycle (lights on: 06:00 h-18:00 h). All behavioural testing was during the light phase. Rats were placed on a restricted diet regimen, where they were fed once a day with approximately 20 g of standard laboratory chow at the completion of testing procedures (16:00 h-18:00 h). For these animals, water was freely available except during testing. All animal use procedures were performed in accordance with the principles of the Canadian Council on Animal Care (CCAC).

Methods: A total of 72 rats were used in the psilocybin experiment. The basic test design involved training animals to lever press for a food reward (45 mg Bioserv pellets) under a progressive ratio (PR) schedule in which the number of responses required to achieve a pellet increases for successive reinforcers. The progressive ratio was derived from the equation ratio=[5×e^{(0.2×reinforcer #)}−5], and was as follows: 2, 4, 6, 9, 12, 15, 20, 25, 32, 40, 50, 62, 77, 95, 118, 145, etc. The break point, which is the primary measure for this task, was determined when the rat failed to earn a pellet in 20 minutes, providing a quantitative measurement for motivation.

Subjects were administered psilocybin (0.025, 0.05, 0.1 mg/kg) or vehicle control according to Latin square design, in which each animal received each treatment over repeated test sessions with 2-3 day intervals between each testing cycle. Subjects were also subgrouped into low responders (N=24) based on having the lowest average break point measure recorded over 7 days prior to testing and a separate analysis was conducted on this cohort.

Statistical analysis: Data are presented as means±SEM. Data from the progressive ratio task was analysed by one way (treatment) repeated measures ANOVA (Statistica Version 11, Statsoft Inc. [2012]). In the event of a significant main effect, post-hoc comparisons were carried out with Dunnett's test for comparison of drug treatment to vehicle control. Both study cohorts of “All” and “Low responders” were analysed separately. In all cases the accepted level of significance was P<0.05. Effect sizes for group mean differences were also calculated using partial eta squared (Statistica Version 11, Statsoft Inc. [2012]). In addition a correlational analysis (parametric: Pearson R; non-parametric: Spearman R) was conducted at each dose level to look at the relationship between baseline (vehicle) response and magnitude of change in break point and lever press following each psilocybin dose.

Results: Across the total of 72 rats used in this experiment, there was no main effect of treatment on number of lever press (F3,210=1.9, P=0.1, NS; ηₚ²=0.03), or session duration (F3,210=1.2, NS; ηₚ²=0.02). Although a main effect of treatment was recorded for break point (F3,210=3.3, P=0.02; ηₚ²=0.05), no treatment group was significantly different to vehicle. Separation of the 72 rats based on the lowest average break point recorded over the 7 days prior to testing, identified a “low responder” subgroup of N=24 rats. Analysis confined to this “low responder” cohort identified main effects of treatment on lever press (F3,69=4.9, P<0.01; ηₚ²=0.17) and break point (F3,69=5.6, P<0.01; ηₚ²=0.19), which was due to increases in both measures following psilocybin 0.05-0.1 mg/kg pretreatment relative to vehicle control. There was no treatment effect on session duration (F3,69=2.4, P=0.07, NS; ηₚ²=0.09), although a modest trend for this to be increased at the 0.1 mg/kg dose was noted (Veh: 15.1±2.0 min; Psilo 0.1 mg/kg: 18.7±2.5 min).

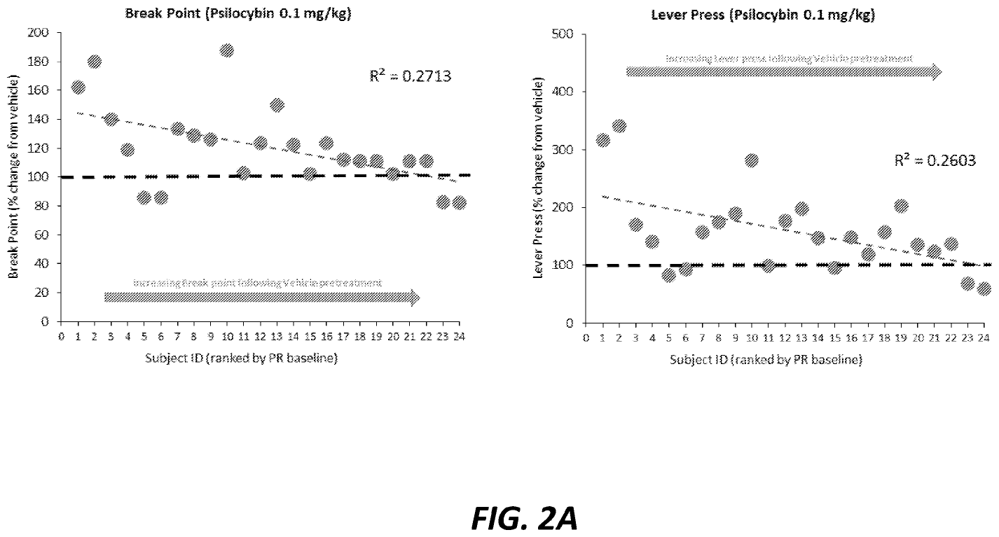

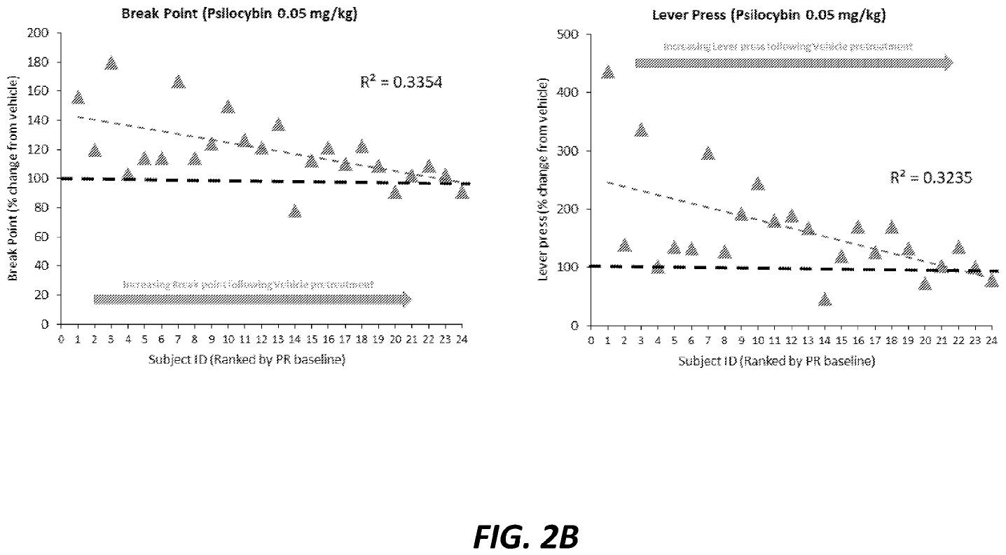

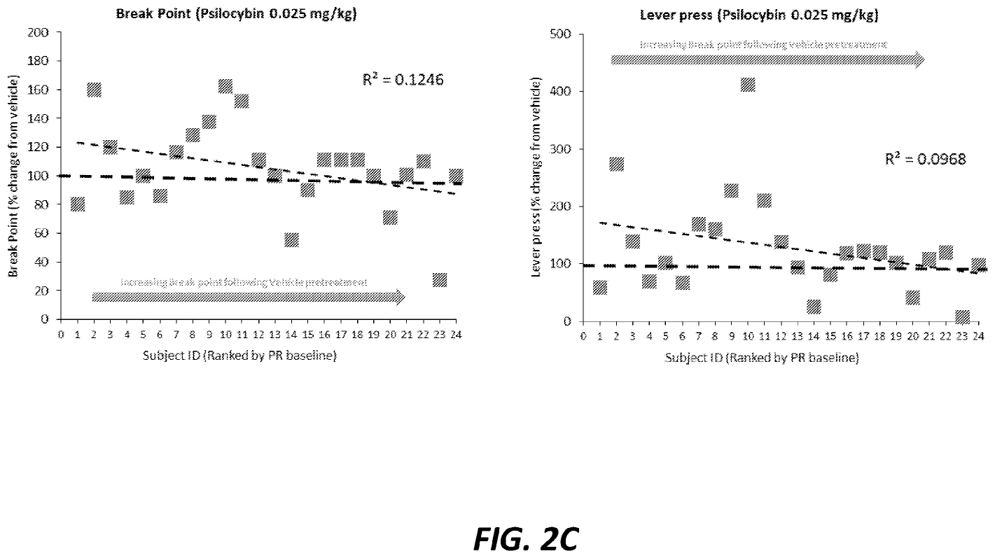

In addition, we examined the relationship between baseline responding and % change following psilocybin pretreatment measured either as break point or lever press. At both the 0.05 mg/kg and 0.1 mg/kg doses there was an inverse correlation in that the largest magnitude of change was seen in the lowest responders, i.e an ascending ranking of the N=24 “low responder” group based on break point, resulted in a diminishing change in response. Both a parametric analysis (Pearson R) and non-parametric analysis (Spearman R) revealed a significant correlation at both 0.05 mg/kg and 0.1 mg/kg doses (P<0.01) (see FIG. 2A-B). At the lowest 0.025 mg/kg dose of psilocybin there was no such correlation (see FIG. 2C).

The identification and improvement in the “low responder” animals indicates utility of the treatment of behavioral and cognitive disorders involving these behaviors, including but not limited to depression, anxiety, apathy and low motivation, attention disorders, disorders of executive function and cognitive engagement, obsessive compulsive disorder, and neurocognitive disorders. A diagnosis of Major Depressive Disorder (MDD) includes symptoms of depressed mood, anhedonia, fatigue/loss of energy (anergia), cognitive deficits including diminished/slowed ability to think or concentrate and feelings of guilt, worthlessness, and suicidal ideation. Therefore endophenotypes related to depression include anhedonia (impaired reward function) amotivation (lack of motivation/purpose) and impaired cognitive function.

### Example 2:5-Choice Reaction Time Task and STAI Assessment in Subjects Dosed with Psilocybin and Identification of “Low Responders”

In the treatment phase of an ascending dosing study conducted in healthy volunteers, 6 subjects of one cohort each received a single oral dose of 3.5 mg psilocybin. Together with the collection of safety and pharmacodynamic data, the subjects were assessed at baseline (pupil diameter, State-Trait Anxiety Inventory (STAI) and Five-Choice Reaction Time Task), as well as at 1 hr post dosing (pupil diameter) and at 2 hr and 4 hr post dosing (Five-Choice Reaction Time Task).

### State-Trait Anxiety Inventory (STAI) Questionnaire

Subjects were assessed at baseline using the STAI Form Y which is a widely used self-report instrument for assessing anxiety in adults (FIG. 14). It includes separate measures of state and trait anxiety. The STAI evaluates the essential qualities of feelings of apprehension, tension, nervousness, and worry, and differentiates between the temporary condition of state anxiety and the more general and longstanding quality of trait anxiety. The STAI state anxiety subscale (STAI-S) asks for feelings at the moment of filling out the questionnaire, while the STAI trait anxiety subscale (STAI-T) asks subjects to indicate how they generally view themselves. Each question is rated on a 4-point scale (not at all, somewhat, moderately so, very much so). The range of possible scores for the STAI Form Y spans from a minimum score of 20 to a maximum score of 80 on both the STAI-T and STAI-S subscales. The STAI-S is the “state” anxiety subscale, while the STAI-T is the “trait,” anxiety subscale.

### 5-Choice Reaction Time Task (RTI)

Subjects were assessed using RTI at baseline, and 2 hr and 4 hr post dosing. RTI is a sensitive measure of psychomotor speed. In this 5-choice reaction time task, subjects hold down a button at the bottom of the screen until a yellow spot appears in 1 of 5 circles at the top of the screen. They must then release the button and touch inside the circle where the yellow spot appeared as quickly as they can. Practice trials are included to familiarize subjects with the task. The key outcome measures for this task are median 5-choice reaction and movement time.

### Pupillometry

Pupillometry was used as an objective physiological pharmacodynamic measure at baseline and at 1 hr post dosing and included maximum pupil dilation (MPD) and time to MPD (TMPD). An electronic pupillometer was used to measure pupil diameter. Data from a series of frames was used in the calculation, and the final display was the weighted average and standard deviation (SD) of the pupil size. Measurements were collected under consistent brighter or daylight (not-dimmed) lighting conditions. For each subject, every effort was made to use the same eye for all assessments throughout the study.

### Results

Subjects with the lowest reaction times in the 5-choice reaction time study demonstrated the best reaction times following dosing at 3.5 mg psilocybin. As shown in Table 1, for example, subject number 1035 scored the lowest reaction time (464) prior to dosing but demonstrated the most improved scores at 2 hr (−3.9%) and 4 hr (−7.1%) post dosing. Subject 1035 had the highest anxiety score (STAI-S and STAI-T) at baseline indicating there was an inverse correlation in that the largest magnitude of change was seen in the lowest responder. This correlation indicates the potential identification of “low responders” based on baseline anxiety scores (STAI-S and STAI-T) and/or reaction time who may respond to treatment with an active agent provided herein.

TABLE 1
Five-Choice Reaction Time Task and STAI Assessment
in Subjects Dosed with 3.5 mg Psilocybin

| | | RTI Median Five- | | | | | | |
|---|---|---|---|---|---|---|---|---|
| | | Choice Reaction Time | | | % change | | | |
| | | | | Dose | Baseline | Baseline | Baseline | Baseline |
| subject ID | Baseline | 2 Hr | 4 Hr | (mg) | to 2 Hr | to 4 Hr | STAI-S | STAI-T |
| 1039 | 332.5 | 343 | 330.5 | 3.5 | 3.2% | −0.6% | 50 | 47 |
| 1038 | 338 | 381 | 330 | 3.5 | 12.7% | −2.4% | 46 | 40 |
| 1040 | 340 | 347 | 346 | 3.5 | 2.1% | 1.8% | 42 | 45 |
| 1033 | 384 | 377 | 377 | 3.5 | −1.8% | −1.8% | 38 | 53 |
| 1037 | 396.5 | 386.5 | 413 | 3.5 | −2.5% | 4.2% | 47 | 45 |
| 1035 | 464 | 446 | 431 | 3.5 | −3.9% | −7.1% | 56 | 53 |

Results from subjects pupillometry measurements at 1 hr post dosing were plotted in relation to 4 hr post dosing movement time (RTI) as seen in FIG. 15. The data shows a trend in subjects post dosing that demonstrates a correlation between 1 hr change in pupil dilation from baseline vs. long term improvement (4 hr from baseline) in movement time. This correlation indicates the potential identification of “low responders” based on pupil dilation and/or movement time who may respond to treatment with an active agent provided herein.

### Example 3: Randomized, Double-Blind Study to Evaluate the Safety, Tolerability, and Efficacy of Psilocybin Oral Solution in Adults with Generalized Anxiety Disorder

The planned study is a 2-part study to assess the safety, tolerability, efficacy, and pharmacokinetics (PK) of 3 mg psilocybin as a treatment for generalized anxiety disorder (GAD). The study will be conducted in 2 parts, Part A and Part B. In both study parts, patients will receive the study drug daily (qd) or every 3 days (q3d).

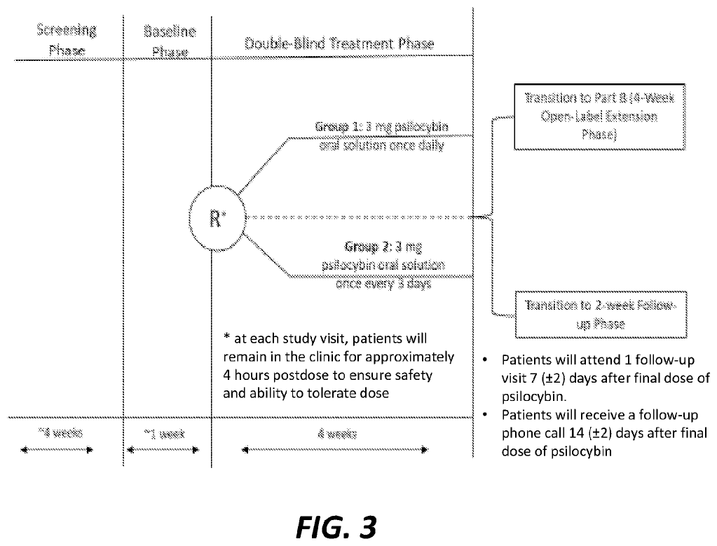

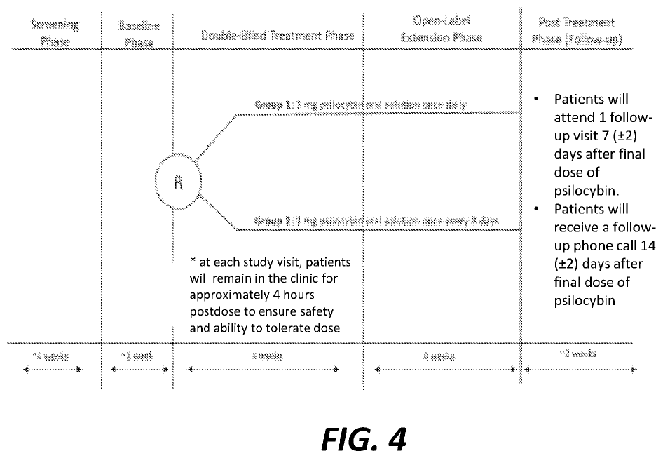

An overview of the study design is provided in FIG. 3 (Part A only) and FIG. 4 (Part A and B). Study assessments and procedures will be performed at the visits and time points outlined in the Schedule of Assessments (Table 2 and Table 3).

Each patient who participates only in Part A will participate in the study for up to ˜11 weeks, including Screening (up to 4 weeks), Baseline (up to 1 week), Double-Blind Treatment Phase (4 weeks), and Follow-up (2 weeks). Each patient who participates in Part A and Part B will participate in the study for up to ˜14 weeks, including Screening (up to 3 weeks), Baseline (up to 1 week), Double-Blind Treatment Phase (4 weeks), Open-Label Extension Study (˜4 weeks), and Post-Treatment/Follow-up Phase (2 weeks).

### Objectives

### Double-Blind Study (Part A):

The primary objective of Part A of the study is to evaluate the safety and tolerability of 3 mg psilocybin in an oral solution administered qd or q3d for 4 weeks in adults who meet criteria for GAD.

The secondary objectives of Part A of the study are to assess the efficacy of 3 mg psilocybin in an oral solution administered qd and q3d for 4 weeks in the treatment of adults who meet criteria for GAD and to assess the PK of 3 mg psilocybin in an oral solution in adults with GAD.

The exploratory objectives of Part A of the study are to evaluate the subjective and cognitive effects of 3 mg psilocybin in an oral solution administered qd and q3d for 4 weeks in adults who meet criteria for GAD, to evaluate the effects of 3 mg psilocybin in an oral solution administered qd and q3d for 4 weeks on quality of life in adults who meet criteria for GAD, as measured by the Quality of Life Enjoyment and Satisfaction Questionnaire-Short Form (Q-LES-Q-SF), and to assess the effects of 3 mg psilocybin in an oral solution administered qd and q3d for 4 weeks on electroencephalogram (EEG) resting state in adults who meet criteria for GAD.

### Open-Label Safety Extension Study (Part B)

The primary objective of Part B of the study is to evaluate the safety and tolerability of 3 mg psilocybin in an oral solution administered qd or q3d for up to a total of 8 weeks in adults who meet criteria for GAD.

The secondary objectives of Part B of the study are to assess the efficacy of 3 mg psilocybin in an oral solution administered qd and q3d for up to a total of 8 weeks in the treatment of adults who meet criteria for GAD.

The exploratory objectives of Part B of the study are to evaluate the subjective and cognitive effects of 3 mg psilocybin in an oral solution administered qd and q3d for up to a total of 8 weeks in adults who meet criteria for GAD, to evaluate the effects of 3 mg psilocybin in an oral solution administered qd and q3d for up to a total of 8 weeks on quality of life in adults who meet criteria for GAD, as measured by the Q-LES-Q-SF, and to assess the effects of 3 mg psilocybin in an oral solution administered qd and q3d for up to a total of 8 weeks on EEG resting state in adults who meet criteria for GAD.

### Methodology

An overview of the study design is provided in FIG. 3 (Part A only) and FIG. 4 (Part A and B). Study assessments and procedures will be performed at the visits and time points outlined in the Schedule of Assessments (Table 2 and Table 3). Each patient who participates only in Part A will participate in the study for up to ˜11 weeks, including Screening (up to 4 weeks), Baseline (up to 1 week), Double-Blind Treatment Phase (4 weeks), and Follow-up (2 weeks). Each patient who participates in Part A and Part B will participate in the study for up to ˜14 weeks, including Screening (up to 3 weeks), Baseline (up to 1 week), Double-Blind Treatment Phase (4 weeks), Open-Label Extension Study (˜4 weeks), and Post-Treatment/Follow-up Phase (2 weeks).

### Double-Blind Study—Part A

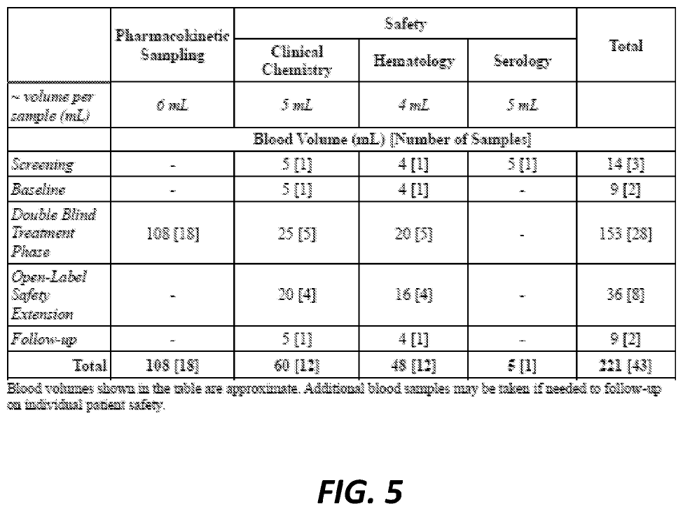

Part A is a randomized, double-blind study designed to evaluate the safety, tolerability, efficacy, and PK of psilocybin in an oral solution in 40 patients with GAD. Part A will include 4 phases: a Screening Phase (up to 4 weeks), a Baseline Phase (up to 1 week), a Double-Blind Treatment Phase (4 weeks), and a Post-treatment/Follow-up Phase (˜2 weeks). An overview of the Study Design is shown in FIG. 5.

At Screening (Visit 1), patients will be asked to provide informed consent, will undergo a standard medical screening, including an abbreviated psychiatric assessment, and will be evaluated for entry into the study. To be eligible at Screening, each patient must have a clinician-rated (GAD-7) score ≥10 and a CGI-S score ≥4. In addition, patients must not currently be receiving regular treatment with an anxiolytic medication (see Table 4 for full list of restricted medications) or must have discontinued their treatment regimen a minimum of 4 weeks prior to baseline.

Following confirmation of eligibility during the Screening Phase, eligible patients will attend an outpatient Baseline Visit (Visit 2) during which all patients will undergo standard safety assessments, complete baseline questionnaires, cognitive assessments, and an EEG. At this visit, patients will also be asked to provide details on their patterns of occasional use (i.e., as needed [prn]) of medication to treat symptoms of anxiety (e.g., benzodiazepines), as well as baseline patterns of recreational drug and alcohol use. To remain eligible at the Baseline Visit, GAD-7 and CGI-S scores must continue to be ≥10 and ≥4, respectively. Eligible patients will be randomized to receive psilocybin oral solution qd or q3d in a 1:1 ratio.

Patients will return to the clinical site within 7 days (±2 days) of the Baseline Visit to enter the 4 week Double-Blind Treatment Phase. During the Double-Blind Treatment Phase, patients will attend 5 clinic visits (Visits 3 to 7), 1 week apart, for trial assessments, with remote contact in between visits (phone call, e-mail, or text message once a week). At each visit, patients will receive a 1-week supply (6 days as the first dose is administered in clinic) of study drug (patients in the q3d treatment group will receive a combination of placebo and active oral solution) that they will be instructed to take qd in the morning.

While at the study visit, patients will be administered their daily dose of psilocybin oral solution (or placebo for patients in the q3d arm) under clinical supervision and will be supervised until at least 4 hours after dosing. Patients will also be required to complete safety assessments and efficacy questionnaires; blood samples for PK assessments and EEG will also be collected at certain visits, as outlined in Table 2.

There will be no dosage adjustments during the Double-Blind Phase; however, patients will be permitted to use concomitant medications as needed, provided that reliable baseline use was previously reported and the medication is approved by the investigator. Patients will be instructed to record all concomitant medication, recreational drug, and alcohol use in their daily diary.

At the end of the final study visit of the Double-Blind Phase (Day 28 or early termination [ET]), patients will enter the Post-Treatment/Follow-up Phase. A Follow-up Visit will be completed 7 (±2) days after final study drug administration and patients will receive a follow-up phone call 14 (±2) days after final study drug administration. Reasonable efforts will be made to ensure continuity of care for patients.

### Open-Label Safety Extension Study (Part B)

Patients who complete the Double-Blind Treatment Phase of the Double-Blind Study (Part A) will be offered the opportunity to continue treatment in an Open-Label Safety Extension Study (Part B) for up to 4 weeks. Patients who sign the informed consent for the Open-Label Safety Extension Study will not be required to participate in the Double-Blind Follow-up Phase and may proceed directly into the Open-Label Safety Extension Study at Visit 7 (Day 28) (FIG. 4).

During the Open-Label Safety Extension Study, patients who chose to continue will return to the study clinic for their regular weekly visits (Visits 8 to 12), 1 week apart, for trial assessments, with remote contact in between visits (phone call, e-mail, or text message once a week). At each visit, patients will receive a 1-week supply (6 days as the first dose is administered in clinic) of study drug (patients in the q3d treatment group will receive a combination of placebo and active oral solution) that they will be instructed to take qd in the morning. While at the study visit, patients will be administered their daily dose of psilocybin oral solution (or placebo for patients in the q3d arm) under clinical supervision. Patients will also be required to complete safety assessments and efficacy questionnaires; EEG will also be collected at certain visits as outlined in Table 3.

There will be no dosage adjustments during the Open-Label Safety Extension Phase; patients will be permitted to continue to use concomitant medications prn, consistent with use in the Double-Blind Treatment Phase. Patients will be instructed to record all concomitant medication, recreational drug, and alcohol use in their daily diary. At the end of the final study visit of the Open-Label Safety Extension (Day 56 or ET), patients will enter the Post-treatment/Follow-up Phase. A Follow-up Visit will be completed 7 (±2) days after final study drug administration and patients will receive a follow-up phone-call 14 (±2) days after final study drug administration. Reasonable efforts will be made to ensure continuity of care for patients.

TABLE 2
Schedule of Assessments - Part A

| | Trial Phase: | | | | | | | | | | | | | | | |
|---|---|---|---|---|---|---|---|---|---|---|---|---|---|---|---|---|
| | Screening | Baseline^{a} | Double-Blind Treatment^{b} | | | | | | | | | | | | | |
| | Week: | | | | | | | | | | | | | | | |
| | −4 to −1 | 1 | 2 to 5 | | | | | | | | | | | | | |
| | Day: | | | | | | | | | | | | | | | |
| | −21 to −2 | −1 | 1 | 2 | 3 | 4 | 5 | 6 | 7 | 8 | 9 | 10 | 11 | 12 | 13 | 14 |
| | Visit: | | | | | | | | | | | | | | | |
| | 1 | 2 | 3 to 7 | | | | | | | | | | | | | |
| In-person | X | X | X | | | | | | | X | | | | | | |
| visit: | | | | | | | | | | | | | | | | |
| Remote | | | | | | | X | | | | | | | X | | |
| contact^{e}: | | | | | | | | | | | | | | | | |
| In-person | X | X | X | | | | | | | X | | | | | | |
| visit: | | | | | | | | | | | | | | | | |
| Remote | | | | | | | X | | | | | | | X | | |
| contact^{e}: | | | | | | | | | | | | | | | | |
| Informed | X | | | | | | | | | | | | | | | |
| consent^{f} | | | | | | | | | | | | | | | | |
| Demographics | X | | | | | | | | | | | | | | | |
| Medical | X | | | | | | | | | | | | | | | |
| history | | | | | | | | | | | | | | | | |
| Prior | X | | | | | | | | | | | | | | | |
| medications | | | | | | | | | | | | | | | | |
| Concomitant | | X | X | X | X | X | X | X | X | X | X | X | X | X | X | X |
| medications^{g} | | | | | | | | | | | | | | | | |
| Physical | X | X | X | | | | | | | X | | | | | | |
| examination^{h} | | | | | | | | | | | | | | | | |
| Height, | X | | | | | | | | | | | | | | | |
| weight, | | | | | | | | | | | | | | | | |
| BMI | | | | | | | | | | | | | | | | |
| HIV, | X | | | | | | | | | | | | | | | |
| Hepatitis | | | | | | | | | | | | | | | | |
| B/C | | | | | | | | | | | | | | | | |
| 12-lead | X | X | X | | | | | | | X | | | | | | |
| ECG | | | | | | | | | | | | | | | | |
| Clinical | X | X | X | | | | | | | X | | | | | | |
| laboratory | | | | | | | | | | | | | | | | |
| tests | | | | | | | | | | | | | | | | |
| Serum | X | X | X | | | | | | | X | | | | | | |
| pregnancy | | | | | | | | | | | | | | | | |
| test^{j} | | | | | | | | | | | | | | | | |
| UDS | X | X | X | | | | | | | X | | | | | | |
| Alcohol | X | X | X | | | | | | | X | | | | | | |
| breath test | | | | | | | | | | | | | | | | |
| DSM-V | X | | | | | | | | | | | | | | | |
| MADRS | X | | | | | | | | | | | | | | | |
| IES-R | X | | | | | | | | | | | | | | | |
| MINI | X | | | | | | | | | | | | | | | |
| DART | X | | | | | | | | | | | | | | | |
| Randomization^{k} | | X | | | | | | | | | | | | | | |
| Daily | | | X | X | X | X | X | X | X | X | X | X | X | X | X | X |
| psilocybin | | | | | | | | | | | | | | | | |
| or placebo | | | | | | | | | | | | | | | | |
| administration | | | | | | | | | | | | | | | | |
| Dispensing | | | X | | | | | | | X | | | | | | |
| of | | | | | | | | | | | | | | | | |
| psilocybin | | | | | | | | | | | | | | | | |
| or placebo | | | | | | | | | | | | | | | | |
| C-SSRS^{l} | X | X | X | | | | | | | X | | | | | | |
| GAD-7 | X | X | X | | | | | | | X | | | | | | |
| HADS | | X | X | | | | | | | X | | | | | | |
| CGI-S | X | X | X | | | | | | | X | | | | | | |
| CGI-I | | | | | | | | | | X | | | | | | |
| STAI | | X | X | | | | | | | X | | | | | | |
| Bowdle | | X | X | | | | | | | X | | | | | | |
| VAS | | | | | | | | | | | | | | | | |
| DSST, | | X | X | | | | | | | X | | | | | | |
| SCWT, | | | | | | | | | | | | | | | | |
| and CFQ | | | | | | | | | | | | | | | | |
| Q-LES-Q- | | X | | | | | | | | | | | | | | |
| SF | | | | | | | | | | | | | | | | |
| PK blood | | | X | | | | | | | | | | | | | |
| sampling^{m} | | | | | | | | | | | | | | | | |
| EEG | | X | | | | | | | | | | | | | | |
| Patient | | | X | X | X | X | X | X | X | X | X | X | X | X | X | X |
| daily diary | | | | | | | | | | | | | | | | |
| AEsⁿ | X | X | X | X | X | X | X | X | X | X | X | X | X | X | X | X |
| Early | | | | | | | | | | | | | | | | |
| Discontinuation | | | | | | | | | | | | | | | | |
| Assessment^{o} | | | | | | | | | | | | | | | | |

| | Trial Phase: | | | | | | | | | | | | | | | |
|---|---|---|---|---|---|---|---|---|---|---|---|---|---|---|---|---|
| | | | | | | | | | | | | | | | Follow-up^{c} | |
| | | | | | | | | | | | | | | | Follow-up | Phone |
| | Double-Blind Treatment^{b} | | | | | | | | | | | | | | visit | call |
| | Week: | | | | | | | | | | | | | | | |
| | 2 to 5 | | | | | | | | | | | | | | 6 | 7 |
| | Day: | | | | | | | | | | | | | | | |
| | 15 | 16 | 17 | 18 | 19 | 20 | 21 | 22 | 23 | 24 | 25 | 26 | 27 | 28 | | |
| | Visit: | | | | | | | | | | | | | | | |
| | 3 to 7 | | | | | | | | | | | | | | 8 and 9 | |
| In-person | X | | | | | | | X | | | | — | | X^{d} | X | |
| visit: | | | | | | | | | | | | | | | | |
| Remote | | | | | X | | | | | | X | | | | | X |
| contact^{e}: | | | | | | | | | | | | | | | | |
| In-person | X | | | | | | | X | | | | — | | X^{d} | X | |
| visit: | | | | | | | | | | | | | | | | |
| Remote | | | | | X | | | | | | X | | | | | X |
| contact^{e}: | | | | | | | | | | | | | | | | |
| Informed | | | | | | | | | | | | | | | | |
| consent^{f} | | | | | | | | | | | | | | | | |
| Demographics | | | | | | | | | | | | | | | | |
| Medical | | | | | | | | | | | | | | | | |
| history | | | | | | | | | | | | | | | | |
| Prior | | | | | | | | | | | | | | | | |
| medications | | | | | | | | | | | | | | | | |
| Concomitant | X | X | X | X | X | X | X | X | X | X | X | X | X | X | X | X |
| medications^{g} | | | | | | | | | | | | | | | | |
| Physical | X | | | | | | | X | | | | | | X | X | |
| examination^{h} | | | | | | | | | | | | | | | | |
| Height, | | | | | | | | | | | | | | | | |
| weight, | | | | | | | | | | | | | | | | |
| BMI | | | | | | | | | | | | | | | | |
| HIV, | | | | | | | | | | | | | | | | |
| Hepatitis | | | | | | | | | | | | | | | | |
| B/C | | | | | | | | | | | | | | | | |
| 12-lead | X | | | | | | | X | | | | | | X | X | |
| ECG | | | | | | | | | | | | | | | | |
| Clinical | X | | | | | | | X | | | | | | X | X | |
| laboratory | | | | | | | | | | | | | | | | |
| tests | | | | | | | | | | | | | | | | |
| Serum | X | | | | | | | X | | | | | | X | X | |
| pregnancy | | | | | | | | | | | | | | | | |
| test^{j} | | | | | | | | | | | | | | | | |
| UDS | X | | | | | | | X | | | | | | X | X | |
| Alcohol | X | | | | | | | X | | | | | | X | X | |
| breath test | | | | | | | | | | | | | | | | |
| DSM-V | | | | | | | | | | | | | | | | |
| MADRS | | | | | | | | | | | | | | | | |
| IES-R | | | | | | | | | | | | | | | | |
| MINI | | | | | | | | | | | | | | | | |
| DART | | | | | | | | | | | | | | | | |
| Randomization^{k} | | | | | | | | | | | | | | | | |
| Daily | X | X | X | X | X | X | X | X | X | X | X | X | X | X | | |
| psilocybin | | | | | | | | | | | | | | | | |
| or placebo | | | | | | | | | | | | | | | | |
| administration | | | | | | | | | | | | | | | | |
| Dispensing | X | | | | | | | X | | | | | | | | |
| of | | | | | | | | | | | | | | | | |
| psilocybin | | | | | | | | | | | | | | | | |
| or placebo | | | | | | | | | | | | | | | | |
| C-SSRS^{l} | X | | | | | | | X | | | | | | X | X | X |
| GAD-7 | X | | | | | | | X | | | | | | X | X | X |
| HADS | X | | | | | | | X | | | | | | X | X | X |
| CGI-S | X | | | | | | | X | | | | | | X | X | |
| CGI-I | X | | | | | | | X | | | | | | X | X | |
| STAI | X | | | | | | | X | | | | | | X | X | |
| Bowdle | X | | | | | | | X | | | | | | X | | |
| VAS | | | | | | | | | | | | | | | | |
| DSST, | X | | | | | | | X | | | | | | X | | |
| SCWT, | | | | | | | | | | | | | | | | |
| and CFQ | | | | | | | | | | | | | | | | |
| Q-LES-Q- | | | | | | | | | | | | | | X | | |
| SF | | | | | | | | | | | | | | | | |
| PK blood | | | | | | | | | | | | | | X | | |
| sampling^{m} | | | | | | | | | | | | | | | | |
| EEG | | | | | | | | | | | | | | X | | |
| Patient | X | X | X | X | X | X | X | X | X | X | X | X | X | X | X | X |
| daily diary | | | | | | | | | | | | | | | | |
| AEsⁿ | X | X | X | X | X | X | X | X | X | X | X | X | X | X | X | X |
| Early | | | | | | | | | | | | | | | X | |
| Discontinuation | | | | | | | | | | | | | | | | |
| Assessment^{o} | | | | | | | | | | | | | | | | |

^{a}All patients will attend an in-person baseline visit in order to complete safety and efficacy assessments prior to beginning treatment. The Baseline Phase will occur within approximately 7 (±2) days of first treatment administration during the Double-Blind Treatment Phase.
^{b}All patients will attend 5 in-person clinic visits (Days 1, 8, 15, 22, and 28) during the Double-Blind Treatment Phase to receive a 1-week supply of study drug (6 days as the first dose is administered in clinic). At each study visit, subjects will receive study drug while supervised and will remain in the clinic for a minimum of 4-hours post-dose for observation and to complete efficacy and safety assessments. On Day 1 and Day 28 subjects will also have blood samples drawn for pharmacokinetic analysis. On all other treatment days, patients will self-administer study drug. Remote contact will be performed weekly for recording of AEs (by phone-call, email, or text message).
^{c}Patients who chose not to enroll in Part B will return for a final safety visit within 7 (±2) days of taking their last dose of psilocybin. A follow-up phone-call will be completed with 14 (±2) days of the last dose of psilocybin.
^{d}All patients will attend an in-person visit on Day 28 (last day of treatment in the Double-Blind Phase). Patients who are interested in continuing treatment will be rolled over into Part B (Open-Label Safety Extension - details of Day 28 procedures for rollover patients are listed in Table 3) of the study, the 4-week Open-Label Extension Phase and will receive their first week supply of study drug at this visit.
^{e}Remote contact may occur via telephone, email, text messaging, or video conferencing, according to the research site and patient's preferences.
^{f}Patients must sign the informed consent form prior to conducting any trial procedures.
^{g}Concomitant medication use will be recorded throughout the study but patients will be questioned on use of concomitant medications at each visit/remote contact.
^{h}Full physical examination at Screening and brief physical examination thereafter.
ⁱAt Screening: height, weight, pulse rate, respiratory rate, blood pressure, and body mass index. At subsequent visits: pulse rate, respiratory rate, and blood pressure only.
^{j}Women of childbearing potential only. Serum pregnancy tests will coincide with clinical laboratory tests.
^{k}Patients will be randomized to receive 3 mg psilocybin oral solution administered daily (qd), or once every 3-days (q3d).
^{l}The “Screening” version will be administered at Screening. The “Since Last Visit” version will be administered at subsequent visits.
^{m}Blood samples will be collected pre-dose and at 0.5, 1, 1.5, 2, 2.5, 3, 3.5, and 4 hours post-dose.
ⁿPatients will be questioned about adverse events using a non-leading question at each visit and remote contact.
^{o}The Early Discontinuation Assessment will be completed for patients who were administered at least 1 dose of psilocybin and who withdraw consent from the trial (i.e., subject decision). This assessment will be used to evaluate the patient's reason(s) for withdrawal. Patients who discontinue early will advance to the Follow-up assessments (Visit 8 and 9).
Abbreviations: AE = adverse event; BMI-body mass index; CFQ = Cognitive Failures Questionnaire; CGI-I = Clinical Global Impression-Improvement; CGI-S = Clinical Global Impression - Severity; C-SSRS = Columbia-Suicide Severity Rating Scale; DART = Diagnostic Assessment Research Tool; DSST = Digit Symbol Substitution Task; EEG = electroencephalogram; ECG = Electrocardiogram; GAD-7 = Generalized Anxiety Disorder scale-7-item version; HADS = Hospital Anxiety and Depression Scale; HIV = Human immunodeficiency virus; IES-R = Impact of Event Scale - Revised; MADRS = Montgomery-Asberg

TABLE 3
Schedule of Events - Part B

| | Trial Phase: | | | | | | | | | | | | | | | | |
|---|---|---|---|---|---|---|---|---|---|---|---|---|---|---|---|---|---|
| | Open-Label Safety Extension^{a} | | | | | | | | | | | | | | | | |
| | Week: | | | | | | | | | | | | | | | | |
| | 6 to 9 | | | | | | | | | | | | | | | | |
| | Day: | | | | | | | | | | | | | | | | |
| | 28^{c} | 29 | 30 | 31 | 32 | 33 | 34 | 35 | 36 | 37 | 38 | 39 | 40 | 41 | 42 | 43 | 44 |
| | Visit: | | | | | | | | | | | | | | | | |
| | 8 to 11 | | | | | | | | | | | | | | | | |
| In-person | X | | | | | | | | X | | | | | | | X | |
| visit: | | | | | | | | | | | | | | | | | |
| Remote | | | | | X | | | | | | | X | | | | | |
| contact^{d}: | | | | | | | | | | | | | | | | | |
| Informed | X | | | | | | | | | | | | | | | | |
| consent^{e} | | | | | | | | | | | | | | | | | |
| Concomitant | X | | X | X | X | X | X | X | X | X | X | X | X | X | X | X | X |
| medications^{f} | | | | | | | | | | | | | | | | | |
| Physical | X | | | | | | | | X | | | | | | | X | |
| examination^{g} | | | | | | | | | | | | | | | | | |
| Vital signs^{h} | | | | | | | | | X | | | | | | | X | |
| Oral | X | | | | | | | | X | | | | | | | X | |
| temperature | | | | | | | | | | | | | | | | | |
| 12-lead | X | | | | | | | | X | | | | | | | X | |
| ECG | | | | | | | | | | | | | | | | | |
| Clinical | X | | | | | | | | X | | | | | | | X | |
| laboratory | | | | | | | | | | | | | | | | | |
| tests | | | | | | | | | | | | | | | | | |
| Serum | X | | | | | | | | X | | | | | | | X | |
| pregnancy | | | | | | | | | | | | | | | | | |
| testⁱ | | | | | | | | | | | | | | | | | |
| UDS | X | | | | | | | | X | | | | | | | X | |
| Alcohol | X | | | | | | | | X | | | | | | | X | |
| breath test | | | | | | | | | | | | | | | | | |
| Daily | X | X | X | X | X | X | X | X | X | X | X | X | X | X | X | X | X |
| psilocybin | | | | | | | | | | | | | | | | | |
| or placebo | | | | | | | | | | | | | | | | | |
| administration | | | | | | | | | | | | | | | | | |
| Dispensing | X^{j} | | | | | | | | X | | | | | | | X | |
| of | | | | | | | | | | | | | | | | | |
| psilocybin | | | | | | | | | | | | | | | | | |
| or placebo | | | | | | | | | | | | | | | | | |
| C-SSRS^{k} | | | | | | | | | X | | | | | | | X | |
| GAD-7 | | | | | | | | | X | | | | | | | X | |
| HADS | | | | | | | | | X | | | | | | | X | |
| CGI-S | | | | | | | | | X | | | | | | | X | |
| CGI-I | | | | | | | | | X | | | | | | | X | |
| STAI | | | | | | | | | X | | | | | | | X | |
| Bowdle | | | | | | | | | X | | | | | | | X | |
| VAS | | | | | | | | | | | | | | | | | |
| DSST, | | | | | | | | | X | | | | | | | X | |
| SCWT, and | | | | | | | | | | | | | | | | | |
| CFQ | | | | | | | | | | | | | | | | | |
| Q-LES-Q-SF | | | | | | | | | | | | | | | | | |
| EEG | | | | | | | | | | | | | | | | | |
| Patient | | X | X | X | X | X | X | X | X | X | X | X | X | X | X | X | X |
| daily diary | | | | | | | | | | | | | | | | | |
| AEs^{l} | | X | X | X | X | X | X | X | X | X | X | X | X | X | X | X | X |
| Early | | | | | | | | | | | | | | | | | |
| Discontinuation | | | | | | | | | | | | | | | | | |
| Assessment^{m} | | | | | | | | | | | | | | | | | |

| | Trial Phase: | | | | | | | | | | | | | |
|---|---|---|---|---|---|---|---|---|---|---|---|---|---|---|
| | | | | | | | | | | | | | Follow-up^{b} | |
| | | | | | | | | | | | | | Follow-up | Phone- |
| | Open-Label Safety Extension^{a} | | | | | | | | | | | | visit | call |
| | Week: | | | | | | | | | | | | | |
| | 6 to 9 | | | | | | | | | | | | 10 | 11 |
| | Day: | | | | | | | | | | | | | |
| | 45 | 46 | 47 | 48 | 49 | 50 | 51 | 52 | 53 | 54 | 55 | 56 | | |
| | Visit: | | | | | | | | | | | | | |
| | 8 to 11 | | | | | | | | | | | | 12 and 13 | |
| In-person | | | | | | X | | | | | | X | X | |
| visit: | | | | | | | | | | | | | | |
| Remote | | X | | | | | | | X | | | | | X |
| contact^{d}: | | | | | | | | | | | | | | |
| Informed | | | | | | | | | | | | | | |
| consent^{e} | | | | | | | | | | | | | | |
| Concomitant | X | X | X | X | X | X | X | X | X | X | X | X | X | X |
| medications^{f} | | | | | | | | | | | | | | |
| Physical | | | | | | X | | | | | | X | X | |
| examination^{g} | | | | | | | | | | | | | | |
| Vital signs^{h} | | | | | | X | | | | | | X | X | |
| Oral | | | | | | X | | | | | | X | X | |
| temperature | | | | | | | | | | | | | | |
| 12-lead | | | | | | X | | | | | | X | X | |
| ECG | | | | | | | | | | | | | | |
| Clinical | | | | | | X | | | | | | X | X | |
| laboratory | | | | | | | | | | | | | | |
| tests | | | | | | | | | | | | | | |
| Serum | | | | | | X | | | | | | X | X | |
| pregnancy | | | | | | | | | | | | | | |
| testⁱ | | | | | | | | | | | | | | |
| UDS | | | | | | X | | | | | | X | X | |
| Alcohol | | | | | | X | | | | | | X | X | |
| breath test | | | | | | | | | | | | | | |
| Daily | X | X | X | X | X | X | X | X | X | X | X | X | | |
| psilocybin | | | | | | | | | | | | | | |
| or placebo | | | | | | | | | | | | | | |
| administration | | | | | | | | | | | | | | |
| Dispensing | | | | | | X | | | | | | | | |
| of | | | | | | | | | | | | | | |
| psilocybin | | | | | | | | | | | | | | |
| or placebo | | | | | | | | | | | | | | |
| C-SSRS^{k} | | | | | | X | | | | | | X | X | X |
| GAD-7 | | | | | | X | | | | | | X | X | X |
| HADS | | | | | | X | | | | | | X | X | X |
| CGI-S | | | | | | X | | | | | | X | X | |
| CGI-I | | | | | | X | | | | | | X | X | |
| STAI | | | | | | X | | | | | | X | X | |
| Bowdle | | | | | | X | | | | | | X | | |
| VAS | | | | | | | | | | | | | | |
| DSST, | | | | | | X | | | | | | X | | |
| SCWT, and | | | | | | | | | | | | | | |
| CFQ | | | | | | | | | | | | | | |
| Q-LES-Q-SF | | | | | | | | | | | | X | | |
| EEG | | | | | | | | | | | | X | | |
| Patient | X | X | X | X | X | X | X | X | X | X | X | X | X | X |
| daily diary | | | | | | | | | | | | | | |
| AEs^{l} | X | X | X | X | X | X | X | X | X | X | X | X | X | X |
| Early | | | | | | | | | | | | | X | |
| Discontinuation | | | | | | | | | | | | | | |
| Assessment^{m} | | | | | | | | | | | | | | |

^{a}All patients will attend 4 in-person clinic visits (Days 36, 43, 50, and 56 [Visits 8, 9, 10, and 11]) during the Open-Label Extension Phase to receive a 1-week supply of study drug (7 days of study drug on Day 28 [to begin Part B dosing on Day 29] and 6 days of study drug at all other visits). At each study visit, subjects will receive study drug while supervised and will remain in the clinic to complete efficacy and safety assessments. On all other treatment days, patients will self-administer study drug. Remote contact will be performed weekly for recording of AEs (by phone-call).
^{b}Patients will return for a final safety visit within 7 (±2) days of taking their last dose of psilocybin. A follow-up phone-call will be completed with 14 (±2) days of the last dose of psilocybin.
^{c}All patients will attend an in-person visit on Day 28 (last day of treatment in the Double-Blind Phase). Patients rolled over into Part B will complete required assessments for the completion of Part A and will receive their lust week supply of study drug for Part B at this visit.
^{d}Remote contact may occur via telephone, email, text messaging, or video conferencing, according to the research site and patient's preferences.
^{e}Patients must sign the informed consent form (on Day 28 of Double-Blind Phase) prior to conducting any trial procedures.
^{f}Concomitant medication use will be recorded throughout the study but patients will be questioned on use of concomitant medications at each visit/remote contact.
^{g}Brief physical examination
^{h}Vital signs will include pulse rate, respiratory rate, and blood pressure only.
ⁱWomen of childbearing potential only. Serum pregnancy tests will coincide with clinical laboratory tests.
^{j}Study drug will be dispensed on Day 28 of the Double - Blind Treatment Phase (Part A)
^{k}The “Screening” version will be administered at Screening. The “Since Last Visit” version will be administered at subsequent visits.
^{l}Patients will be questioned atom adverse events using a non-leading question at each visit and remote contact.
^{m}The Early Discontinuation Assessment will be completed for patients who were administered at least 1 dose of psilocybin and who withdraw consent from the trial (i.e., subject decision). This assessment will be used to evaluate the patient's reason(s) for withdrawal. Patients who discontinue early will advance to the Follow-up assessments (Visit 12 and 13).
Abbreviations: AE = adverse event CFQ = Cognitive Failures Questionnaire; CGI-I = Clinical Global Impression-Improvement; CGI-S = Clinical Global Impression - Severity⁻, C-SSRS = Columbia-Suicide Seventy Rating Scale; DSST = Digit-Symbol Substitution Task; EEG = electroencephalogram; ECG = electrocardiogram; GAD-7 = Generalized Anxiety Disorder scale-7-item version; HADS = Hospital Anxiety and Depression Scale; IES-R = Impact of Event Scale - Raised; MADRS = Montgomery-Asbarg Depression Rating Scale; MINI-Psych = Mini Neuropsychiatric Interview; qd = once daily; q3d = once every 3 days; Q-LES-Q-SF = Quality of Life Enjoyment⁻ and Satisfaction Questionnaire; SCWT = Stroop Colour and Word Task; UDS = urine drug screen; VAS = visual analog scale.

TABLE 4
Restricted Medications

| | | Dosing |
|---|---|---|
| Treatment | Dose | regimen |
| SSRIs (e.g., sertraline, | As recommended for the | Daily use |
| fluoxetine, citalopram, | treatment of anxiety | |
| escitalopram, vortioxetine) | disorders | |
| SNRIs (e.g., duloxetine, | | |
| venlafaxine, desvenlafaxine) | | |
| Mirtazapine | >7.5 mg/day | Daily use |
| Trazodone | >100 mg/day | |
| Tricyclic antidepressants | Doses recommended for | |
| | anxiety | |
| Quetiapine, aripiprazole, | Quetiapine: >50 mg/day | Daily use |
| olanzapine, brexpiprazole, and | Aripiprazole: >2 mg/day | |
| other atypical antipsychotics | Olanzapine: >2.5 mg/day | |
| with anxiolytic properties | Brexpiprazole: >0.5 mg/day | |
| Pregabalin (when administered | >100 mg/day | Daily use |
| at anxiolytic doses) | | |

SNRI = Serotonin and norepinephrine reuptake inhibitor;
SSRI = Selective serotonin-reuptake inhibitor

### Selection of Study Population

The planned sample size is 40 patients with 20 patients randomized into each treatment group in the Double-Blind Treatment Phase.

### Double-Blind Treatment Study (Part A)

### Inclusion Criteria

Each patient must meet the following inclusion criteria to be eligible for participation in Part A of this trial:
1. Must provide written informed consent prior to the initiation of any protocol-specific procedures.
2. Male and female adults, between 18 and 60 years of age, inclusive.
3. Meets DSM-V criteria for a primary diagnosis of GAD at Screening (duration of diagnosis ≥1 year, based on self-report), confirmed using the MINI and the DART.
4. Clinician-rated GAD-7 score ≥10 at Screening (Visit 1).
5. CGI-S score ≥4 at Screening (Visit 1). Females must be non-pregnant and non-lactating and must fulfil at least one of the following:
   a. Be surgically sterile for a minimum of 6 months (achieved through hysterectomy, oophorectomy, or bilateral salpingectomy; note that tubal ligation is not considered a method of permanent sterilization).
   b. Post-menopausal for a minimum of 1 year (confirmed by follicle-stimulating hormone test).
   c. Agree to avoid pregnancy and use a medically acceptable method of contraception with male sexual partners from at least 30 days prior to the study until 30 days after the study has ended (last study procedure).
   d. Medically acceptable methods of contraception include any of the following:
      i. Double-barrier methods (e.g., male condom, spermicide with diaphragm or spermicide with cervical cap)
      ii. Oral contraceptives; hormonal patch, implant or injection; or hormonal or non-hormonal intrauterine device. The male partner should use, at all times, a male condom with spermicide, should the female Patient choose to use any of these methods
      iii. Complete abstinence, should it be in line with the Patient's preferred and usual lifestyle.
6. Males who are able to father children must agree to use medically acceptable methods of contraception during the study and for 30 days after the last study drug administration. If a patient's partner should become pregnant during his participation in the study and for 30 days after he has completed his last study drug administration, the patient must inform study staff immediately. Medically acceptable methods of contraception include:
   a. Using a condom with a female partner of child-bearing potential who is using oral contraceptives; hormonal patch, implant or injection; hormonal or non-hormonal intrauterine device; or diaphragm or cervical cap with spermicide
   b. Complete abstinence, should it be in line with the patient's preferred and usual lifestyle.
7. Males must refrain from sperm donation from clinic admission to at least 30 days after the last dose of study drug.
8. Must be able to speak, read, and understand English sufficiently to allow completion of all study assessments.
9. Must be willing to comply with the requirements and restrictions of the study.

### Exclusion Criteria

A patient will not be eligible to participate in Part A of this trial if any one of the following exclusion criteria is met:
1. Personal history of schizophrenia, bipolar affective disorder, delusional disorder, paranoid disorder, moderate or severe panic disorder, moderate or severe social anxiety disorder, schizoaffective disorder, moderate or severe obsessive-compulsive disorder, anorexia nervosa, bulimia nervosa, moderate or severe post-traumatic stress disorder (as assessed by the IES-R), moderate or severe personality disorder (Cluster B and Cluster C only), moderate or severe MDD (as assessed by the MINI, DART, and scores on the MADRS).
2. History or presence of any clinically significant cardiac, endocrine, hematologic, hepatic, immunologic, metabolic, urologic, pulmonary, neurologic, dermatologic, renal, or other disease at Screening, which, in the opinion of the investigator, would jeopardize the safety of the patient or the validity of the study results.
3. Recently initiated non-pharmacological treatment (e.g., Cognitive Behavioral Therapy, psychotherapy) with a psychologist or health care professional (i.e., <4 weeks prior to Screening). Patients who are began a stable non-pharmacological treatment regimen >4 weeks prior to Screening will be permitted. Patients currently stable on treatment will not be permitted to modify or introduce new elements to their existing treatment regimen while enrolled in the study.
4. Currently taking pharmacological treatment for GAD or depressive symptoms on a daily basis as outlined in Table 4. Patients who discontinue pharmacological treatment within a minimum of 4 weeks or more of baseline will be permitted to enroll in the study. Sporadic, prn use of anxiolytics will be permitted; however, patients will be required to document all medication use prior to study enrollment.
5. Clinically significant abnormality on ECG, including a QT interval corrected for heart rate (Bazett; QTcB interval) of >440 milliseconds in males and >460 milliseconds in females.
6. History of allergies to the investigational product or excipients.
7. History of seizures, family history of seizures, history of head trauma, history of neurosurgery, or close family history of idiopathic generalized epilepsy or other congenital epilepsies.
8. Positive for hepatitis B virus surface antigen, hepatitis C virus antibody, or human immunodeficiency virus (HIV) antibody.
9. Positive urine drug screen at Screening.
10. Current or history of moderate or severe drug or alcohol use disorder (excluding caffeine and nicotine) within the past 2 years, or lifetime history of participation in a drug rehabilitation program (other than treatment for smoking cessation).
11. Has used CNS drugs with perception-altering properties (e.g., ketamine, lysergic acid diethylamide [LSD], phencyclidine [PCP], 3,4 methylenedioxymethamphetamine [MDMA], mescaline, psilocybin, tryptamine derivatives, or ring-substituted amphetamines with perception-altering effects) on more than 1 occasion for therapeutic or non-therapeutic purposes (i.e., for psychoactive effects) within the past 6 months. History of single or episodic use will not be considered exclusionary.
12. History of suicidal ideation in the past 12 months or active/current suicidality, based on C-SSRS results.
13. Is currently using an investigational drug or device or has used such in the 30 days prior to receiving study drug.
14. Female patient is pregnant, breastfeeding, or has had a recent positive pregnancy test.
15. Patient, in the investigator's opinion, is unsuitable for clinical study participation.
   Criteria for Randomization into Double-Blind Phase

Each patient must meet the following criteria for enrollment into the Double-Blind Treatment Phase, (1) Clinician-rated GAD-7 score ≥10 at Baseline (Visit 2), and (2) CGI-S score ≥4 at Baseline (Visit 2).

### Open-Label Safety Extension (Part B)

### Inclusion Criteria

Inclusion criteria for the open-label safety extension (Part B) include (1) Completed Double-Blind Treatment Phase of the study, and (2) Signed ICF for Open-Label Safety Extension Phase.

### Exclusion Criteria

Exclusion criteria for the open-label safety extension (Part B) include clinically significant symptoms, medical conditions, or other circumstances which, in the opinion of the investigator, would preclude compliance with the protocol, adequate cooperation in the study, or obtaining informed consent, or may prevent the patient from safely participating in the study.

### Study Restrictions

In addition to the inclusion/exclusion criteria described herein, the patient must agree to abide by the following study restrictions:
1. Patients will be required to abstain from alcohol for 48 hours prior to Screening. Abstinence will be confirmed via a breath alcohol test.
2. Patients will be asked to abstain from strenuous physical activity for 48 hours prior to Screening and each inpatient study visit.
3. Patients will be asked to refrain from driving, operating machinery, or engaging in hazardous activities for approximately 4 hours after taking study treatment. If this is not possible due to patient schedule, an alternate dosing time may be proposed, with approval by the investigator.
4. Patients will be required to abstain from blood donation during the study and for 30 days after the Follow-Up Visit.

### Treatment Administration

The following treatments will be administered during the Part A and Part B of the study:
1. Psilocybin oral solution, 3 mg, administered daily (0.5 mg/mL solution).
2. Psilocybin oral solution, 3 mg, administered every 3 days (0.5 mg/mL solution) and placebo solution (0.2% sucralose) administered on all other days.

Double-blind fixed oral doses of psilocybin 3 mg will be administered either qd or q3d for 4 weeks in the Double-Blind Treatment Study (Part A) and for an additional 4 weeks in the Open-Label Safety Extension Study (Part B).

No dosage adjustments will be permitted during Part A or Part B.

Concomitant medications will be permitted.

Psilocybin 0.5 mg/ml oral solution contains the drug substance psilocybin dissolved in a 0.2% w/v aqueous sucralose solution, which is used as a taste masking agent. The dosage form will be produced in campaigns based on the patient recruitment rate across the clinical sites. Batch manufacturing of the oral solution will be performed within production cleanrooms. The solution will be pre-filled into amber, 10 mL oral dosing syringes with a syringe tip-cap to provide a 3 mg dose of psilocybin. The pre-filled syringes will be packaged in heat-sealed tamper-evident, clear, LDPE bags with a vertical perforation and stored at 2-8° C.

The first dose will be administered to the patient at the clinic site visit. The doses for the remainder of the week (until the following weekly clinic site visit) will be provided to the patient in a secure combination lock box (which will be stored in their refrigerator). This process will repeat weekly until the clinical study is complete. Each LDPE bad of study drug will be clearly labeled with study-specific information meeting all the applicable regulatory requirements.

The placebo will be identical to the investigational product with the omission of psilocybin.

At each Study Visit, 1 dose of study drug will be administered in a controlled clinical setting. Paramedic/Registered Nurse/Registered Practical Nurse/Medical Assistant staff will be onsite and trained to deal specifically with potential expected AEs. Medications will be available for administration if needed to treat hypertension, anxiety, or acute psychosis.

Study treatment will be administered using a pre-filled oral dosing syringe. Following dosing, study staff will fill the syringe with approximately 10 mL of room-temperature water and administer it to patients. Patients will be required to drink all of the study solution and an additional 200 mL of room-temperature water within 90 seconds, under the supervision of study personnel.

At each study visit, patients will be dispensed a 1-week (6-day) supply of study drug. Each dose of study drug will be provided in a pre-filled oral syringe packaged in a clear, tamper-evident, heat-sealed bag. The week's supply of study drug will be further packaged in a secure lock-box that will be required to be refrigerated at the patient's home.

Patient's will be instructed to take their dose of study drug at approximately the same time daily. Following dosing, patients will be required to fill the syringe with approximately 10 mL of room-temperature water and swallow the water to ensure all drug has been administered. Patients will be required to log dosing details (i.e., time of dosing and whether the dose was completed) in their daily diary.

The study will evaluate a 3 mg dose of psilocybin, administered both qd and q3d. Patients will be administered their first dose and doses at each weekly visit under clinical supervision and monitored for a minimum of 4 hours after dosing.

In addition to qd dosing, the current study is investigating a q3d regimen in order to empirically evaluate and compare psilocybin dosing protocols that have been previously endorsed but not consistently tested at very low dose levels.

During the Double-Blind Treatment Study (Part A), patients randomized to the qd treatment group will receive psilocybin 3 mg once-daily, with approximately 24 hours between doses. Patients randomized to the q3d treatment groups will receive psilocybin 3 mg once every 3 days, with approximately 72 hours between doses. To maintain blinding, on non-psilocybin dosing days, these patients will receive placebo to match the psilocybin oral solution, so that patients in this treatment group will be dosed daily either with psilocybin or placebo.

During the Open-Label Extension Study (Part B), patients randomized to the qd treatment group will receive psilocybin 3 mg once-daily, with approximately 24 hours between doses. Patients randomized to the q3d treatment groups will receive psilocybin 3 mg once every 3 days, with approximately 72 hours between doses. To maintain blinding (until database lock), on non-psilocybin dosing days, these patients will continue to receive placebo to match the psilocybin oral solution, so that patients in this treatment group will be dosed daily either with psilocybin or placebo.

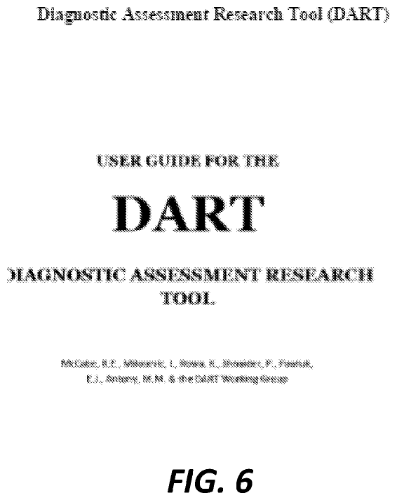

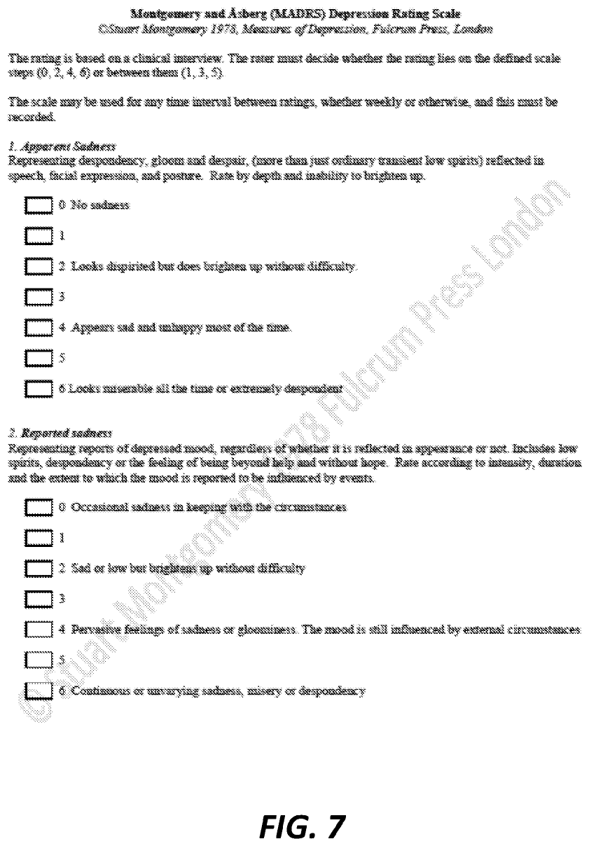

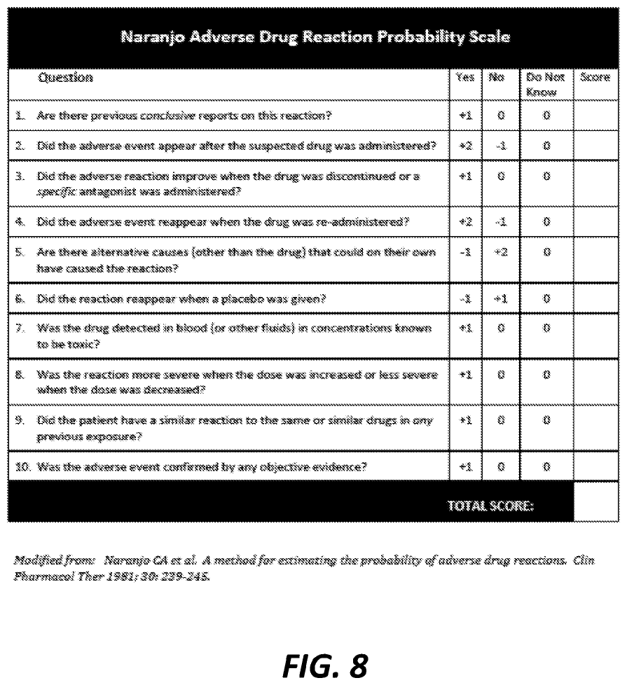

For both treatment groups, no fasting or dietary requirements are required for the trial (Part A and Part B). All study assessments will be performed at the visits and timepoints outlined in the Schedule of Assessments and Schedule of Events (Table 2 and Table 3). Various tools used to carry out the study assessments are provided at FIGS. 6-13. Table 5 shows the clinical laboratory assessments performed during the trial.

TABLE 5
Clinical Laboratory Assessments

| Hematology | Biochemistry | Urinalysis |
|---|---|---|
| Hematocrit | Sodium | Color |
| Hemoglobin | Potassium | pH |
| Red blood cell count | Glucose (random) | Specific gravity |
| Total ad differential | Creatinine | Ketones |
| (absolute) white | Total protein | Protein |
| blood cell count | Blood urea nitrogen | Glucose |
| Platelets | Albumin | Bilirubin |
| | Total bilirubin | Nitrite |
| | Alanine transferase | Urobilinogen |
| | Aspartate trans feast | Occult blood |
| | Lactic dehydrogenase | Microscopic examination |
| | Gamma-glutamyl | of sediment, only if |
| | transferase | urinalysis dipstick results |
| | Alkaline phosphatase | are abnormal |
| | Creatine phosphokinase | |
| | Follicle stimulating | |
| | hormone^{a} | |

^{a}Postmenopausal women at Screening only.

### Study Conduct

### Informed Consent

The nature of the study and its risks and benefits will be explained to the participant by the investigator or designated study personnel. The participant must provide written informed consent on an ethics-approved informed consent form (ICF), prior to performing any study-related procedures.

### Demographics

The following demographics will be recorded: age (birthdate), sex, race, and ethnicity.

### Medical History

The complete medical history will include histories of acute, chronic, or infectious disease; surgical or oncologic histories; and any reported conditions affecting major body systems (including psychiatric disorders). All findings on medical history will be evaluated by the investigator for clinical significance. Medical histories will be obtained according to the site's standard operating procedures.

### Medication History and Recreational/Alcohol Drug Use

All medications (prescription and non-prescription, herbal medications/natural health products, or investigational drugs) taken by the patients during the 30 days prior to Screening will be recorded in the source documentation as medication history.

A history of all drugs used for recreational/non-medicinal purposes (i.e., psychoactive effects) in the past 6 months will be collected. History, including drug preference (i.e., drug of choice) and frequency of use, will be collected using reported drug names, by drug class (e.g., cannabinoids, depressants, dissociative anesthetics, hallucinogens, opioids and morphine derivatives, stimulants). Frequency and amount of alcohol use will also be recorded.

DSM-V modules will be included as a part of the recreational drug/alcohol use history and used to screen for alcohol and substance use disorders.

### Mini International Neuropsychiatric Interview (MINI)

The Mini International Neuropsychiatric Interview (MINI) is a short diagnostic structured interview developed to explore 17 disorders according to DSM-V diagnostic criteria. It is fully structured to allow administration by non-specialized interviewers. In order to keep it short it focuses on the existence of current disorders. For each disorder, 1 or 2 screening questions rule out the diagnosis when answered negatively. Probes for severity, disability or medically explained symptoms are not explored symptom-by-symptom.

### Diagnostic Assessment Research Tool (DART)

The DART is a modular, semistructured diagnostic interview for assessing mental disorders based on DSM-V diagnostic criteria. It is suitable for use in research and clinical settings and designed to allow interviewers to select disorder-specific modules relevant to their participant or patient populations. Each module can be administered independently or in combination with other modules based on positively endorsed items on a self-report screener or at the interviewer's discretion. Completion of a module in isolation may be useful for certain research purposes; however, given the overlap of symptoms across disorders, completion of all DART modules may facilitate differential diagnosis. Some DART modules are disorder-specific, whereas other modules contain disorders that are grouped together and are referred to as sections for greater efficiency in diagnosing (i.e., Depressive Disorders, Posttraumatic Stress Disorder and Acute Stress Disorder, Eating Disorders, and Risk Assessment). DART is intended to be used by interviewers who are familiar with psychopathology and who have a basic understanding of the DSM-V and diagnostic assessment.

### The Montgomery-Åsberg Depression Rating Scale (MADRS)

The MADRS is a 10-item diagnostic questionnaire used to measure the severity of depressive episodes in patients with mood disorders.

### Impact of Event Scale-Revised (IES-R)

The IES-R is a 22-item self-report measure that assesses subjective distress caused by traumatic events. Patients will be asked to identify a specific stressful life event and then indicate how much they were distressed or bothered during the past 7 days by each “difficulty” listed. Items are rated on a 5-point scale ranging from 0 (“not at all”) to 4 (“extremely”). The IES-R yields a total score (ranging from 0 to 88).

### Safety Assessments

### Adverse Events (AEs) and Serious Adverse Events (SAEs)

The investigator and research site staff are responsible for the detection, documentation, classification, reporting, and follow-up of events meeting the definition of an adverse event (AE) or severe adverse event (SAE). All AEs will be recorded following informed consent (at Screening) until the end of the Follow-up period of the study.

### Adverse Events

An AE is any untoward medical occurrence in a patient or clinical investigation patient administered a pharmaceutical product and may not necessarily have a causal relationship with the administered treatment. An AE can therefore be any unfavorable and unintended sign (including a clinically significant laboratory abnormality), symptom, or disease temporally associated with the use of an investigational product, whether or not related to the investigational product. During the study, an AE can also occur outside the time that the investigational product(s) was given (e.g., during a washout period). Pre-existing conditions, diseases, or disorders are not considered AEs unless there is a change in intensity, frequency, or quality.

### Serious Adverse Events and Serious Unexpected Adverse Events

An SAE is any untoward medical occurrence that at any dose (a) results in death, (b) is life-threating (at the time of the event), (c) requires inpatient hospitalization or prolongation of existing hospitalization, (d) results in persistent or significant disability/incapacity, or (e) is a congenital anomaly/birth defect.

Important medical events that may not result in death, be life-threatening, or require hospitalization may be considered a serious adverse drug experience when, based upon appropriate medical judgment, they may jeopardize the study participant and may require medical or surgical intervention to prevent one of the outcomes listed in the definition. Examples of such medical events include allergic bronchospasm requiring intensive treatment in an emergency room or at home, blood dyscrasias or convulsions that do not result in inpatient hospitalization, or the development of drug dependency or drug abuse.

A serious and unexpected AE is an SAE that is not identified in nature, intensity, or frequency in the risk information set out in the Investigator's Brochure/Product Monograph of the drug.

Any SAE experienced by a trial patient—expected or unexpected, irrespective of relationship to trial treatments, including death due to any cause—will be reported to the sponsor or designee by the investigator within 24 hours of learning of the event. Information regarding the SAE will be transmitted to the sponsor or designee, according to the instructions and contact information provided in the safety management plan. The sponsor or designee assumes responsibility for appropriate reporting of AEs to the regulatory authorities. The sponsor or designee will also report to the investigator all SAEs that are unlisted and associated with the use of the trial medication. The investigator (or sponsor/designee, where required) must report these events to the appropriate IRB that approved the protocol. Follow-up evaluations for SAEs will also be reported to the sponsor or designee.

### Adverse Events of Special Interest (AESIs)

There are 4 general categories of AESIs that will be collected:
1. Drug Abuse, Dependence, Withdrawal and Substance-Related Disorders Associated with the Study Drug (e.g., diversion, abuse, misuse, psychological dependence)
2. Perceptual Disturbances/Psychotomimetic Effects (e.g., hallucination, dissociation, confusional state)
3. Mental and Cognitive Impairment (e.g., disturbance in attention, psychomotor skills impaired)
4. Anxiety Symptoms (e.g., insomnia, panic attack)

In addition to the above listed AESI categories, headache, suicidal thoughts, and thoughts of self-harm will also be considered AESIs.

A complete list of relevant AESIs (i.e., preferred terms) will be provided to each site in a separate training manual. Investigators and relevant research site personnel will receive training in the recognition and reporting of AESIs, including further guidance on when such events may need to be reported as SAEs. For all AESIs, additional commentary from the investigator will be required in the Case Report Form (CRF) or other study-specific document, including, where possible, the patient's verbatim description, in order to construct narratives of the events.

The continued participation of patients with AESIs in the trial will be assessed on a case-by-case basis and should be discussed with the medical monitor or designee.

### Clinical Laboratory Abnormalities and Other Abnormal Assessments

Abnormal clinical laboratory findings (e.g., clinical chemistry, hematology, or urinalysis) or other abnormal assessments (e.g., from vital signs or ECGs), judged as clinically significant by the investigator will be recorded as AEs or SAEs, if they meet the definitions provided previously. Abnormal laboratory or other findings present at baseline that significantly worsen following the start of the study will be reported as AEs or SAEs. However, abnormal findings present at the start of the study that do not worsen will not be reported as AEs or SAEs, unless the investigator or designee judges them as more severe than expected for the patient's condition.

### Classification of Adverse Event Intensity and Causality

For each recorded AE or SAE, the investigator must make an assessment of intensity based on the following criteria: (a) Mild: An event that is usually transient and may require only minimal treatment or therapeutic intervention. The event does not generally interfere with usual activities of daily living. (b) Moderate: An event that is alleviated with additional specific therapeutic intervention. The event interferes with usual activities of daily living, causing discomfort but poses no significant or permanent risk of harm to the participant. (c) Severe: An event that requires intensive therapeutic intervention. The event interrupts usual activities of daily living or significantly affects clinical status. The event poses a significant risk of harm to the participant and hospitalization may be required.

The investigator must make an assessment of causality based on the following criteria to determine the relationship between the AE/SAE and study drug including (i) unrelated, (ii) unlikely, (iii) possible, or (iv) probable.

Unrelated: Clearly and incontrovertibly due only to extraneous causes (e.g., disease, environment, etc.) and does not meet the criteria for study drug relationship listed under probable, possible, or unlikely.

Unlikely:
a. Does not follow a reasonable temporal sequence from administration of the study drug.
b. May readily have been produced by the participant's clinical state, environmental, or toxic factors, or other modes of therapy administered to the participant.
c. Does not follow a known pattern of response to the study drug.
d. Does not reappear or worsen when the study drug is re-administered.

Possible: The connection to the study drug appears unlikely but cannot be ruled out with certainty
a. Follows a reasonable temporal sequence from administration of the study drug.
b. May have been produced by the participant's clinical state, environmental, or toxic factors, or other modes of therapy administered to the participant.
c. Follows a pattern of response to the suspected study drug.

Probable: The connection to study drug can be made with a high degree of certainty:
a. Follows a reasonable temporal sequence from administration of the study drug.
b. Cannot be reasonably explained by the known characteristics of the participant's clinical state, environmental, or toxic factors, or other modes of therapy administered to the participant.
c. Disappears or decreases upon cessation or reduction in dose (note that there are important exceptions when an AE or SAE does not disappear upon discontinuation of the study drug, yet drug relatedness clearly exists, e.g., bone marrow depression or tardive dyskinesias).
d. Follows a known pattern of response to the suspected study drug.
e. Reappears upon re-challenge.

### Follow-Up of Adverse Events and Serious Adverse Events

All unresolved AEs will be followed for a minimum of 14 days after the participant's final study visit (Visit 6 [Part A] or Visit 10 [Part B]), unless the investigator's judgment dictates otherwise, the event has resolved or stabilized before the 14-day period, or the patient is lost to follow-up.

All AEs that result in discontinuation and SAEs will be followed until the event resolves, stabilizes (according to the judgment of the investigator), returns to a baseline value (if a baseline value is available), or can be attributed to agents other than the study drug or to factors unrelated to study conduct.

When it becomes unlikely that any additional information can be obtained (e.g., patient or health care practitioner refuses to provide additional information, the patient is lost to follow-up), the investigator or designee will ensure that the follow-up includes any pertinent supplemental investigations (e.g., laboratory tests or investigations, histopathological examinations or consultation with other health care professionals) to elucidate the nature and/or causality of the AE or SAE.

Investigators are not obligated to actively seek AEs or SAEs in former study participants that occur following the Follow-up period. However, if the investigator learns of any AE or SAE at any time after a participant has been discharged from the study and the event is considered as reasonably related to the study drug, the investigator will notify the designee.

For each recorded AE or SAE, the investigator must make an assessment of outcome at the time of last observation, the outcomes include (a) fatal: the participant has died, (b) resolved: the AE or SAE has ended, (c) resolved with sequelae: the AE or SAE has ended by changes are noted from baseline, (d) unresolved—ongoing: the AE has not ended and is ongoing at the end of the reporting period (i.e., 14 days after the final Follow-up visit) and the investigator deems that further follow up is not medically required, or (e) unresolved—lost to follow: lost to follow-up after repeated unsuccessful attempts to contact the participant.

### Pregnancy

If a participant becomes pregnant during the study or for 14 days after the final inpatient Follow-up visit (Visit 6 [Part A] or Visit 10 [Part B]), the investigator will report the pregnancy to the designee within 24 hours of learning of the event. Any participant who becomes pregnant during the study will be immediately withdrawn from the study. Given that the effects of the study drug on sperm have not been fully elucidated, any pregnancy in the partner of a male participant in the study will be reported or designee within 24 hours of learning of the event. Follow-up information will be obtained where possible (with the consent of the participant or their partner) regarding the course and outcome of the pregnancy, including any post-natal sequelae in the infant.

### Clinical Laboratory Assessments

Blood and urine samples will be collected, processed, and shipped according to the research site's standard operating procedures and instructions from the safety laboratory. All clinical laboratory data will be reviewed by the investigator for clinical significance.

Blood volumes required per draw, laboratory test, and study part are available. Additional laboratory samples may be taken at the discretion of the investigator if the results of any tests fall outside reference ranges or clinical symptoms necessitate testing to ensure safety. Specific hematology, biochemistry, and urinalysis assessments are listed in Table 5. In addition to the clinical laboratory tests, pregnancy testing for the presence of β-human chorionic gonadotropin in serum or urine will be performed for all women of childbearing potential. Results of serum or urine pregnancy tests will be reported and determined to be negative prior to study continuation.

A blood sample for a serology panel testing for hepatitis B surface antigen, anti-hepatitis C antibodies, and HIV will be performed for all patients. Only patients with negative viral serology tests will be eligible for the study. Positive results will be managed according to local regulatory requirements and the site's standard operating procedures.

### Urine Drug Screen and Breath Alcohol Testing

Urine drug screens will test for the following drugs of abuse: tetrahydrocannabinol (THC), oxycodone and other opioids, amphetamines, cocaine, and benzodiazepines. The opioid panel will include buprenorphine, norbuprenorphine, codeine, hydrocodone, hydromorphone, methadone, morphine, oxycodone and oxymorphone. Breath alcohol testing will be performed according to the site's standard operating procedures

### Vital Signs

Vital signs will consist of blood pressure (systolic and diastolic blood pressure, mmHg), pulse rate (beats per minute), and respiratory rate (breaths/min). Oral temperature (° C.) will also be taken at some time points, as specified in Table 2 and Table 3. Vital signs will be collected while sitting, following a rest period of at least 3 minutes. The investigator will review all vital signs findings for clinical significance. Any findings meeting the investigator's or sponsor/designee's criteria for clinical significance will be recorded as AEs or SAEs as appropriate. Clinically significant (CS) vital signs findings at Screening may affect patient eligibility for entry into the trial.

### 12-Lead Electrocardiograms

12-Lead ECGs will be performed after the patient has been resting in a supine or semi-supine position for at least 3 minutes. The ECG variables will include ventricular heart rate and PR, QRS, QT, QTcB and/or QTcF intervals. The investigator will review all ECG findings for clinical significance. Any findings meeting the investigator's or sponsor/designee's criteria for clinical significance will be recorded as AEs or SAEs as appropriate. CS ECG findings at Screening may affect patient eligibility for entry into the trial.

### Physical Examination

A complete physical examination assessing the patient's overall health and physical condition will be performed at Screening, and a brief physical examination (examination of heart, lungs, abdomen, and legs) will be performed thereafter. The investigator will review all physical examination findings for clinical significance. Any findings meeting the investigator's or sponsor/designee's criteria for clinical significance will be recorded as AEs or SAEs as appropriate. CS physical examination findings at Screening may affect patient eligibility for entry into the trial. Height, weight, and BMI will be assessed as described in Table 2 and Table 3.

### Columbia-Suicide Severity Rating Scale

The C-SSRS tracks all suicidal events and provides a summary of suicidal ideation and behavior. It assesses the lethality of attempts and other features of ideation (frequency, duration, controllability, reasons for ideation, and deterrents), all of which are significantly predictive of completed suicide.

Two versions of the C-SSRS will be used in this trial: the Baseline/Screening version (Lifetime) and the Since Last Visit version. The Screening/Lifetime version will be administered at Visit 1 (Screening), and the Since Last Visit version will be administered at all subsequent assessments.

A validated telephone or tablet/device-based C-SSRS assessment will be used in this trial. The investigator will have access to the patient's results after completion of the Screening assessment in order to determine patient eligibility. The investigator will receive immediate notification of any high-risk responses and will be responsible for management of the patient (e.g., discontinuation of participation and referral to appropriate follow-up care).

### Patient Daily Diary

Patients will be required to complete a daily electronic diary that will record treatment compliance, i.e., patients will be required to indicate whether they have taken their daily dose of study drug. In addition, the daily diary will record, at minimum, use of concomitant medications, alcohol and recreational drug-use. Additional open-ended questions on AEs and other subjective effects may also be included. Details of the patient diary will be included in the study specific manual.

### Data Safety Monitoring Board

A DSMB will be commissioned for this clinical trial. This committee will be comprised of 3-4 clinical experts in anxiety disorders and/or clinical trial conduct. The primary responsibilities of the DSMB will be to review and evaluate the safety data during the course of the clinical trial and make appropriate recommendations regarding the conduct of the clinical trial to the Sponsor (e.g., safety of 3 mg dose). The DSMB procedures will be described in a separate DSMB manual. The intention is that the DSMB will first meet once the first 10 enrolled patients have completed 2 weeks of qd or q3d dosing with oral psilocybin, and then again once 20 patients have completed 2 weeks of dosing. In addition, the DSMB will meet on an ad hoc basis should serious or severe TEAEs occur.

### Efficacy and Other Assessments

### Generalized Anxiety Disorder Scale

The GAD-7 is a brief measure for instrument for monitoring and measuring the severity of GAD, based on the GAD diagnostic criteria described in the DSM-IV. The GAD-7 assessment asks patients to evaluate their level of symptoms over the last 2 weeks. The instrument is 7 questions long and each question rates the frequency of the symptoms, which factors into the severity index. The GAD-7 is widely used and well validated. Each item is rated on a scale from 0 to 3, a higher score indicates higher levels of anxiety symptoms. Scores of 5 to 9 indicate mild symptom severity, scores of 10 to 14 indicate moderate severity, and scores ≥15 indicate severe cases. A copy of the scale is presented in FIG. 9.

### Hospital Anxiety and Depression Scale

The HADS is a 14-item scale, with 7 items to assess depressive symptoms and 7 items to assess anxiety and takes between 2 to 5 minutes to complete. Each item is rated on a scale from 0 to 3. Scores of 8 to 10 indicate borderline abnormal cases, and scores from 11 to 21 indicate abnormal cases. A copy of the scale is presented in FIG. 10.

### Clinical Global Impressions Scale

The CGI was developed to provide a brief, stand-alone assessment of the clinician's view of the patient's global functioning prior to and after initiating a study medication. The CGI provides an overall clinician-determined summary measure that considers all available information, including patient history, psychosocial circumstances, symptoms, behavior, and the impact of the symptoms on ability to function. The CGI comprises 2 companion 1-item measures evaluating the following: (a) severity of psychopathology from 1 to 7 (CGI-S) and (b) change from the initiation of treatment on a similar 7-point scale (CGI-I). CGI forms can be completed in less than a minute by an experienced rater.

### State-Trait Anxiety Inventory (STAI)

The STAI Form Y is a widely used self-report instrument for assessing anxiety in adults. It includes separate measures of state and trait anxiety. The STAI evaluates the essential qualities of feelings of apprehension, tension, nervousness, and worry, and differentiates between the temporary condition of state anxiety and the more general and longstanding quality of trait anxiety. The STAI-S asks for feelings at the moment of filling out the questionnaire, while the STAI-T asks patients to indicate how they generally view themselves. Each question is rated on a 4-point scale (not at all, somewhat, moderately so, very much so). The range of possible scores for the STAI Form Y spans from a minimum score of 20 to a maximum score of 80 on both the STAI-T and STAI-S subscales.

### Bowdle Visual Analog Scale

The Bowdle VAS consists of 13 items for which the patient is asked to rate his or her feelings over the prior 30 minutes. Each VAS is scored from 0 to 100, with 0 reflecting “not at all” and 100 reflecting “extremely.” Lower individual and overall scores indicate fewer psychedelic effects. Two composite scores are generated, Bowdle VAS External Perceptions composite score reflects a misperception of an external stimulus or a change in the awareness of the patient's surroundings (Items 1, 2, 3, 5, 6, and 7) and Bowdle VAS Internal Perceptions composite scores, which reflect inner feelings that do not correspond with reality (Items 4, 8, 9, 10, and 12). The individual items of the questionnaire are listed in the figures. A copy of the scale is depicted in FIG. 11.

### Quality of Life Enjoyment and Satisfaction Questionnaire-Short Form

The Q-LES-Q-SF is designed to measure a patient's satisfaction and enjoyment in different areas of daily functioning. The original scale (Q-LES-Q) consists of 93 questions, which were grouped into 8 subscales on the basis of expert clinical opinion: physical health, subjective feelings, leisure time activities, social relationships, work, school/coursework, household duties, and general activities. The abbreviated version (Q-LES-Q-SF) consists of 14 items derived from the long form's general activities subscale, plus 2 questions about medication and overall life satisfaction (Stevanovic, 2011). A copy of the questionnaire is depicted in FIG. 12.

### Cognitive Battery—Digit-Symbol Substitution Task, Stroop Colour and Word Test, and Cognitive Failures Questionnaire

A short battery of cognitive tests focusing on selective attention (i.e., the processes that allow an individual to select and focus on particular input for further processing while simultaneously suppressing irrelevant or distracting information), memory, and executive function will be administered at the timepoints outlined in Table 2 and Table 3. The battery will include the DSST, which requires the integrity of executive function, processing speed, attention, spatial perception, and visual scanning, and the SCWT, a task that assesses the ability to inhibit cognitive interference, as well as attention, processing speed, and cognitive flexibility. In addition, the battery will include the CFQ, a self-report measure of cognitive errors experienced in daily life, capturing perceived difficulties with forgetfulness, distractibility, and thinking blunders. A copy of the cognitive failures questionnaire is depicted in FIG. 13.

### Electroencephalogram

EEGs will be recorded with an EEG device containing 32 electrodes. The EEG will be digitized continuously. During EEG pre-processing, repetitive artifacts such as electromyography, 60 Hz line noise, electrode discontinuity, EKG, and ocular artifacts, including blinks, saccades, and lateral eye movements, will be removed. EEG will be recording as resting-state eyes open (lasting about 5 mins), resting-state eyes-closed (lasting about 5 mins). EEG will be assessed as described in Table 2 and Table 3.

### Drug Concentration Measurements

During the Double-Blind Study, venous blood samples will be collected to determine the plasma concentrations of psilocin at the times outlined in the Schedule of Assessments (Table 2). Samples will be collected, processed, and shipped according to the site's standard operating procedures and instructions from the sponsor or bioanalytical laboratory. The bioanalytical lab will remain blinded, and all samples will be analyzed.

1-37. (canceled)

38. A method of assessing an individual's responsiveness to an active ingredient, said method comprising:
(a) identifying or measuring a state of the individual by performing an evaluation on the individual;
(b) assessing the identified or measured state; and
(c) determining the measured state is indicative of responsiveness to an active agent based at least in part on the assessing in step (b).

39. The method of claim 38, wherein the method additionally comprises identifying a biomarker indicative of responsiveness to an active agent based at least in part on the assessing in step (b).

40. The method of claim 38, further comprising treating the individual by administering to the individual the active agent.

41. The method of claim 38, wherein:
(i) the state of the individual comprises a physiological, neurological, psychological, metabolic, or biologic state or a combination thereof;
(ii) the state of the individual is measured or identified by a brain activity, a heart test, a visual test, an auditory test, a biological sample, patient reporting, functional data, body temperature, food intake, metabolic rate, perspiration, hydration, salivation, pupil dilation, breathing rate, pulse rate, skin colour, or skin temperature;
(iii) the identifying or measuring a state comprises measuring reaction and/or movement time in the individual;
(iv) the identifying or measuring a state is performed by administering a questionnaire to the individual;
(v) the identifying or measuring a state is performed through measurement of pupil dilation in the individual; or
(vi) a combination of two or more of (i)-(v).

42. A method of assessing an individual's responsiveness to an active ingredient, said method comprising:
(a) identifying or measuring a biomarker of the individual by performing an evaluation on the individual;
(b) assessing the biomarker; and
(c) determining the biomarker is indicative of responsiveness to an active agent based at least in part of the assessing in step (b).

43. The method of claim 42, further comprising treating the individual by administering to the individual the active agent.

44. The method of claim 42, wherein:
(i) the biomarker is used to identify high or low responding individuals;
(ii) the biomarker is used to identify treatable and non-treatable individuals;
(iii) the biomarker is used to identify individuals that would benefit most from treatment with the active agent;
(iv) the biomarker is used to identify and/or predict personalized medical treatment with the active agent; or
(v) a combination of two or more of (i)-(iv).

45. The method of claim 38, wherein assessing comprises predicting a therapeutic effectiveness of treatment of the individual with the active agent.

46. The method of claim 39, wherein the treating is for treatment of a brain disease, disorder or condition of the individual or for treatment or reduction of incidence of an episode associated with a brain disease, disorder or condition of the individual.

47. The method of claim 43, wherein the treating is for treatment of a brain disease, disorder or condition of the individual or reduction of incidence of an episode associated with a brain disease, disorder or condition of the individual.

48. The method of claim 46, wherein the active agent is one or more 5-HT receptor agonist.

49. The method of claim 46, wherein the active agent is:
(i) formulated for administration at a dose comprising about 0.5 mg to about 5 mg of the active agent or about 3 mg of the active agent;
(ii) for administration to the individual once daily or once every 3 days, and/or for a period of 28 days or less or for at least 28 days;
(iii) formulated for oral administration;
(iv) formulated in a solution at a concentration of about 0.05 mg/mL to about 1 mg/mL, or at a concentration of about 0.5 mg/L; or
(v) a combination of two or more of (i)-(iv).

50. The method of claim 38, wherein the evaluation is performed by the individual or by a health care provider, and/or wherein the evaluation is performed using an application or a wearable device.

51. The method of claim 38, wherein the identifying in step (a) is performed prior to any administration of the active agent to the individual, or wherein a test dose of the active agent has been administered to the individual prior to step (a).

52. The method of claim 38, wherein prior to step (a), the method further comprises obtaining a biological sample from the individual.

53. The method of claim 52, wherein the identifying step comprises measuring the biological sample obtained from the subject.

54. The method of claim 52, wherein the biological sample comprises serum, plasma, whole blood, urine, or the like.

55. The method of claim 38, wherein two or more of the steps are performed simultaneously or sequentially.
